(19) United States
(12) Patent Application Publication
(10) Pub. No.: US 2025/0327794 A1
(43) Pub. Date: Oct. 23, 2025

(54) FLUOROMETRIC METHODS FOR DETECTION OF ALLERGIC SENSITIVITY TO ENVIRONMENTAL, FOODSTUFF, AND MEDICAMENT STIMULANTS

(71) Applicant: CELL SCIENCE SYSTEMS CORPORATION, Deerfield Beach, FL (US)

(72) Inventors: Yen-Chih HUANG, Cooper City, FL (US); Roger Davis DEUTSCH, Highland Beach, FL (US)

(73) Assignee: CELL SCIENCE SYSTEMS CORPORATION, Deerfield Beach, FL (US)

(21) Appl. No.: 18/872,254

(22) PCT Filed: Jun. 7, 2023
(86) PCT No.: PCT/US2023/068045

§ 371 Date: Dec. 5, 2024

Related U.S. Application Data
(60) Provisional application No. 63/350,359, filed on Jun. 8, 2022.

(51) Int. Cl. G01N 33/50

(52) U.S. Cl. CPC G01N 33/5047; G01N 33/5038

(57) ABSTRACT

The present disclosure relates, in part, to flow cytometric methods for detecting or measuring the response of a subject to an exogenous stimulant, such as environmental, foodstuff, and medicament allergens.

### CROSS-REFERENCE TO RELATED APPLICATIONS

This application claims the benefit of U.S. Provisional Patent Application No. 63/350,359, filed Jun. 8, 2022, the disclosure of which is incorporated by reference herein in its entirety.

### 1. FIELD

The present disclosure relates, in part, to flow cytometric methods for detecting or measuring the response of a subject to an exogenous stimulant, such as environmental, foodstuff, and medicament allergens.

### 2. SUMMARY

In certain aspects, provided are methods for detecting or measuring a response of a subject to an exogenous stimulant via fluorometric measurement.

In certain aspects, provided are methods for determining or predicting an immune reaction of a subject to an exogenous stimulant via fluorometric measurement. In some embodiments, said predicted immune reaction of the subject to the exogenous stimulant is immune reaction type and/or immune reaction severity.

In certain aspects, provided are methods for determining or predicting the compatibility of a subject with an exogenous stimulant via fluorometric measurement.

In particular embodiments, said methods may comprise the use of fluorometric measurement of the amount of oxidation-sensitive fluorophore-treated granulocytes in a biological sample from the subject after contact of the granulocytes with the exogenous stimulant ex vivo, wherein the method further comprises contacting the oxidation-sensitive fluorophore-treated granulocytes with one or more binding moieties that bind to a basophil, eosinophil, or neutrophil, and wherein the one or more binding moieties are detectable by flow cytometry, and wherein an increase in intensity of fluorescence from an oxidized form of the oxidation-sensitive fluorophore as measured by flow cytometry after contact of the oxidation-sensitive fluorophore-treated granulocytes with the exogenous stimulant as compared to a reference control indicates a response, an immune reaction, or compatibility of the subject to/with the exogenous stimulant.

In certain aspects, provided are methods for predicting or identifying an immune reaction in a subject to an exogenous stimulant, comprising: (i) obtaining a biological sample from the subject; (ii) isolating a population of leukocytes from the blood sample of (i); (iii) diffusing into the population of leukocytes of (ii) an amount of oxidation-sensitive fluorophore, and contacting the population with the exogenous stimulant, and one or more binding moieties selected from: (a) an anti-neutrophil binding moiety that binds to a neutrophil cell surface marker; (b) an anti-eosinophil binding moiety that binds to a eosinophil cell surface marker; or (c) an anti-basophil binding moiety that binds to a basophil cell surface marker, wherein the one or more binding moieties are detectable by flow cytometry; (iv) isolating via flow cytometry one or more populations of neutrophils, eosinophils, and/or basophils from the leukocytes of (iii) based on cell surface marker positivity or negativity and/or side scattering profile; (v) measuring via fluorometric measurement the level of an oxidized form of an oxidation-sensitive fluorophore in the one or more populations of (iv); (vi) comparing the level of an oxidized form of an oxidation-sensitive fluorophore of (v) with a level of an oxidized form of an oxidation-sensitive fluorophore of a reference control as measured via fluorometric measurement, wherein the reference control is prepared from the same or substantially the same one or more populations of (iv) as in (v); and (vii) identifying the one or more populations of cells in (vi) having higher level of the oxidized form of the oxidation-sensitive fluorophore compared to the level of the oxidized form of the oxidation-sensitive fluorophore of the reference control.

In certain aspects, provided are methods for detecting the activation of a neutrophil, eosinophil, or basophil cell population in a biological sample of a subject following exposure to an exogenous stimulant, comprising: (i) contacting biological sample with the exogenous stimulant and an oxidation-sensitive fluorophore; (ii) detecting via flow cytometry the cell surface expression of one or more neutrophil, eosinophil, or basophil cell surface markers in the leukocyte population of the biological sample; (iii) detecting degranulation of said neutrophil, eosinophil, or basophil population by detecting oxidation of an oxidation-sensitive fluorophore into an oxidized form of said fluorophore; and (iv) concluding that the neutrophil, eosinophil, or basophil population that oxidizes the oxidation-sensitive fluorophore into the oxidized form are activated.

In certain embodiments of the methods as described herein, the oxidation-sensitive fluorophore is selected from dihydrorhodamine (e.g., dihydrorhodamine 123 (DHR 123)), dihydroethidine (DHE), dichlorodihydrofluorscein (DCFH2), or a reduced fluorescein derivative. In certain embodiments, the oxidized form of the oxidation-sensitive fluorophore is selected from rhodamine (e.g., rhodamine 123 (RH 123)), 2-hydroxyethidium, dichlorofluorescein, or an oxidized fluorescein derivative.

In certain aspects, the method further comprises staining the oxidation-sensitive fluorophore-treated granulocytes to determine viability of the granulocytes. In certain aspects, the method further staining the oxidation-sensitive fluorophore-treated granulocytes to determine positive extracellular trap formation. In some embodiments, the method further comprises propidium iodide (PI) staining or contact with an anti-citrullinated histone H3 (H3cit) antibody.

In certain embodiments, the oxidation-sensitive fluorophore-treated granulocytes are basophils and are contacted with one or more binding moieties that each bind to a different target selected from 2D7, CD9, CD11a, CD11b, CD13, CD15, CD16, CD16/32, CD22, CD25, CD32, CD33, CD38, CD43, CD45, CD49b, CD63, CD69, CD88 (C5a receptor), CD123 (IL3Ra), CD125, CD154 (CD40 ligand), CD192 (CCR2), CD203c, CD218 (IL-18R), CD282 (TLR2), CD284 (TLR4), CD286 (TLR6), CD294 (CRTH2), CD281 (TLR1), CD289 (TLR9), C/EBP alpha, CRTH-2, FceR1, or GATA-2.

In certain embodiments, the oxidation-sensitive fluorophore-treated granulocytes are eosinophils and are contacted with one or more binding moieties that each bind to a different target selected from CD9, CD11b, CD13, CD15, CD16, CD16/32, CD24, CD32, CD33, CD35, CD43, CD45, CD49d, CD63, CD64, CD66b, CD116, CD123, CD125, CD126, CD170 (SiglecF), CD193 (CCR3), CD244, EMR1, FceR1, Siglec-8.

In certain embodiments, the oxidation-sensitive fluorophore-treated granulocytes are neutrophils and are contacted with one or more binding moieties that each bind to a different target selected from Calprotectin (S100A8/A9), CD10, CD11b, CD13, CD15, CD16, CD16/32, CD17, CD18, CD24, CD32, CD33, CD35, CD43, CD44, CD49d, CD63, CD66a, CD66b, CD66c, CD66d, CD89, CD93, CD112 (Nectin-2), CD114 (G-CSFR), CD116, CD123, CD157, CD177, CD181 (CXCR1), CD193, CD281 (TLR1), CD282 (TLR2), CD284 (TLR4), CD286 (TLR6), CD289 (TLR9), or Ly-6G (Gr-1).

In further embodiments, the oxidation-sensitive fluorophore-treated granulocytes are contacted with an antibody that binds to CD63. In some embodiments, a granulocyte (e.g., basophil, eosinophil, neutrophil) is determined to be activated when it is CD63(+).

In certain embodiments, fluorometric measurement is performed via flow cytometry or plate reader. In certain embodiments, the biological sample is a blood sample. In certain embodiments, the cell surface marker is a neutrophil cell surface marker, eosinophil cell surface marker, and/or basophil cell surface marker. In certain embodiments, the cell surface marker is selected from CD11b, CD16, CD33, CD63, CD123, CD193, or CD203c. In certain embodiments, the granulocyte and/or activated granulocyte is selected from a basophil, eosinophil, or neutrophil. In certain embodiments, the antibody detectable by flow cytometry specifically binds to a basophil, eosinophil, or neutrophil. In certain embodiments, the oxidation-sensitive fluorophore is DHR 123.

In various aspects of the method as described herein, the exogenous stimulant is selected from an environmental stimulant, foodstuff stimulant, or medicament stimulant. In certain embodiments, the environmental stimulant selected from pollen, plant, insect, dust mite, cockroach, animal, venom, mold, latex, metal, vitamin, or mineral. In certain embodiments, the foodstuff stimulant is selected from a seed, nut, egg, dairy product, oil, condiment, fruit, vegetable, cereal, grain, legume, meat, wheat, soy, seafood, herb, or spice. In certain embodiments, the medicament stimulant is selected from a drug compound, vaccine or component thereof, adjuvant, or pharmaceutical excipient.

In certain embodiments of a method as described herein, the biological sample of the subject comprises a biological fluid. In certain embodiments, the biological fluid comprises an intravascular biological fluid, interstitial biological fluid, or intracellular biological fluid.

In another aspect, provided is the use of any one of the methods as described herein ahead of or for the treatment of a disease or disorder in the subject, for example, as a modified therapy. In certain embodiments, a modified therapy comprises standard therapy for a particular disease or disorder that lacks one or more exogenous stimulants determined or predicted to cause an immune response in the subject.

In another aspect, provided are methods involving the use oxidation-sensitive fluorophores together with flow cytometry and/or microplate analysis for detection of immune response, prediction of immune reaction type and/or severity, or compatibility of a subject to/with an exogenous stimulant by ex vivo testing of a biological sample from the subject.

In another aspect, provided are kits and articles of manufacture comprising any of the molecules, reagents, or compositions described herein.

### 3. BRIEF DESCRIPTION OF THE DRAWINGS

The foregoing summary, as well as the following detailed description of specific embodiments of the present application, will be better understood when read in conjunction with the appended drawings. It should be understood, however, that the application is not limited to the precise embodiments shown in the drawings.

FIG. 1 depicts a flow chart of an exemplary method for determining granulocyte activation in a biological sample following contact with an exogenous stimulant, based on using oxidation-sensitive fluorophore in flow cytometry and/or microplate analysis.

FIG. 2 depicts a schematic representation of an exemplary method for determining granulocyte activation in a biological sample following contact with an exogenous stimulant.

FIGS. 3A-3D depict rhodamine 123 (RH 123) fluorescent intensity detected by BD Accuri C6 flow cytometer channel 1 (FL1-A). FIG. 3A shows the negative control (cells only). FIG. 3B shows the histogram of cells loaded with DHR 123 only. FIG. 3C shows the histogram of cells loaded with DHR 123 and stimulated with PMA to generate RH 123 fluorescence. FIG. 3D shows the overlap plot for data of FIGS. 3A-3C.

FIGS. 4A-4B depict overlayed M1 region lymphocytes and RH 123 fluorescence following DHR 123 oxidation by monocytes and granulocytes. FIG. 4A shows a single parameter histogram of lymphocyte population negative for CD33 staining (channel 3; FL3-A). The majority of M1 region cells are lymphocytes. FIG. 4B shows RH 123 intensity (channel 1; FL1-A). The green fluorescent signal intensity spanned from 10⁴ to 10⁶, the averaged negative control intensity was about 2×10³.

FIGS. 5A-5F depict histogram plots showing time course change of RH 123 fluorescent intensity in channel 1 (FL1-A). FIG. 5A shows cells loaded with DHR 123 only. FIGS. 5B-5F show cells loaded with DHR 123 and stimulated with PMA for 15, 20, 25, 30, and 40 minutes, respectively.

FIGS. 6A-6F depict density plots of control cells and cells stained with single color fluorophores after compensation. FIGS. 6A-6C show after-compensation density plots of control group (cells only). FIGS. 6D-6F show cells stained with single color: CD16-PE (FIG. 6D), CD-193-Perp-Cy5.5 (FIG. 6E), and CD123-APC (FIG. 6F) antibodies at channel 2 (FL2-A), channel 3 (FL3-A), and channel 4 (FL4-A), respectively, after compensation.

FIGS. 7A-7D depict density plots for analysis of eosinophil cell populations.

FIGS. 8A-8T depict time course study of DHR 123 flow cytometry blood testing using RH 123 (Channel 1; FL1-A), CD16 (Channel 2; FL2-A), CD193 (Channel 3; FL3-A), and CD123 (Channel 4; FL3-A). FIGS. 8A-8D show plots of the cell only control (1 hour). FIGS. 8E-8H show plots of cells loaded with DHR 123 only (1 hour). FIGS. 8I-8L show plots of cells loaded with DHR 123 and treated with PMA for 1 hours. FIGS. 8M-8P show plots of cells loaded with DHR 123 and treated with PMA for 2 hours. FIGS. 8Q-8T show plots of cells loaded with DHR 123 and treated with PMA for 3 hours.

FIGS. 9A-9J depict density plots and histograms of DHR 123 and PI flow cytometry blood testing following exposure to dust mite stimulant for 0.5 hours using RH 123 (Channel 1; FL1-A). FIG. 9A and FIG. 9F show density and histogram plots, respectively, of cell only control. FIG. 9B and FIG. 9G show density and histogram plots, respectively, of the group of cells loaded with DHR 123 only. FIG. 9C and FIG. 9H show density and histogram plots, respectively, of the group of cells loaded with DHR 123 and treated with PMA. FIG. 9D and FIG. 9I show density and histogram plots, respectively, of the group of cells loaded with DHR 123 and dust mite solution (2 μL/100 μL blood). FIG. 9E and FIG. 9J show density and histogram plots, respectively, of the group of cells loaded with DHR 123 and dust mite solution (6 μL/100 μL blood). All groups were incubated with or without stimulant for 0.5 hours.

FIGS. 10A-10J depict density plots and histograms of DHR 123 and PI flow cytometry blood testing following exposure to dust mite stimulant for 1.5 hours using RH 123 (Channel 1; FL1-A). FIG. 10A and FIG. 10F show density and histogram plots, respectively, of cell only control. FIG. 10B and FIG. 10G show density and histogram plots, respectively, of the group of cells loaded with DHR 123 only. FIG. 10C and FIG. 10H show density and histogram plots, respectively, of the group of cells loaded with DHR 123 and treated with PMA. FIG. 10D and FIG. 10I show density and histogram plots, respectively, of the group of cells loaded with DHR 123 and dust mite solution (2 μL/100 μL blood). FIG. 10E and FIG. 10J show density and histogram plots, respectively, of the group of cells loaded with DHR 123 and dust mite solution (6 μL/100 μL blood). All groups were incubated with or without stimulant for 1.5 hours.

FIGS. 11A-11J depict density plots and histograms of DHR 123 and PI flow cytometry blood testing following exposure to dust mite stimulant for 2.5 hours using RH 123 (Channel 1; FL1-A). FIG. 11A and FIG. 11F show density and histogram plots, respectively, of cell only control. FIG. 11B and FIG. 11G show density and histogram plots, respectively, of the group of cells loaded with DHR 123 only. FIG. 11C and FIG. 11H show density and histogram plots, respectively, of the group of cells loaded with DHR 123 and treated with PMA. FIG. 11D and FIG. 11I show density and histogram plots, respectively, of the group of cells loaded with DHR 123 and dust mite solution (2 μL/100 μL blood). FIG. 11E and FIG. 11J show density and histogram plots, respectively, of the group of cells loaded with DHR 123 and dust mite solution (6 μL/100 μL blood). All groups were incubated with or without stimulant for 2.5 hours.

FIGS. 12A-12J depict density plots and histograms of DHR 123 and PI flow cytometry blood testing following exposure to dust mite stimulant for 2.5 hours using PI (channel 3, FL3-A). FIG. 12A and FIG. 12F show density and histogram plots, respectively, of cell only control. FIG. 12B and FIG. 12G show density and histogram plots, respectively, of the group of cells loaded with DHR 123 only. FIG. 12C and FIG. 12H show density and histogram plots, respectively, of the group of cells loaded with DHR 123 and treated with PMA. FIG. 12D and FIG. 12I show density and histogram plots, respectively, of the group of cells loaded with DHR 123 and dust mite solution (2 μL/100 μL blood). FIG. 12E and FIG. 12J show density and histogram plots, respectively, of the group of cells loaded with DHR 123 and dust mite solution (6 μL/100 μL blood). All groups were incubated with or without stimulant for 2.5 hours.

FIGS. 13A-13H depict density plots of DHR 123 and PI flow cytometry blood testing following exposure to ragweed pollen, milk, cucumber, and basil stimulants for 2.5 hours using RH 123 (Channel 1; FL1-A). FIGS. 13A-13H show density plots of RH 123 (channel 1; FL1-A) for: cell only control (FIG. 13A); cells loaded with DHR 123 only (FIG. 13B); cells treated with DHR 123 and PMA (FIG. 13C); cells treated with DHR 123 and ragweed pollen (2.5 μL/100 μL blood) (FIG. 13D); cells treated with DHR 123 and ragweed pollen (5 μL/100 μL blood) (FIG. 13E), cells treated with DHR 123 and milk (5 μL/100 μL blood) (FIG. 13F); cells treated with DHR 123 and cucumber (5 μL/100 μL blood) (FIG. 13G), and cells treated with DHR 123 and basil (5 μL/100 μL blood) (FIG. 13H). All groups were incubated with or without stimulant for 2.5 hours.

FIGS. 14A-14H depict histogram plots of DHR 123 and PI flow cytometry blood testing following exposure to ragweed pollen, milk, cucumber, and basil stimulants for 2.5 hours using RH 123 (Channel 1; FL1-A). FIGS. 14A-14H show histogram plots of RH 123 (channel 1; FL1-A) for: cell only control (FIG. 14A); cells loaded with DHR 123 only (FIG. 14B); cells treated with DHR 123 and PMA (FIG. 14C); cells treated with DHR 123 and ragweed pollen (2.5 μL/100 μL blood) (FIG. 14D); cells treated with DHR 123 and ragweed pollen (5 μL/100 μL blood) (FIG. 14E), cells treated with DHR 123 and milk (5 μL/100 μL blood) (FIG. 14F); cells treated with DHR 123 and cucumber 5 μL (FIG. 14G), and cells treated with DHR 123 and basil (5 μL/100 μL blood) (FIG. 14H). All groups were incubated with or without stimulant for 2.5 hours.

FIGS. 15A-15H depict density plots of DHR 123 and PI flow cytometry blood testing following exposure to ragweed pollen, milk, cucumber, and basil stimulants for 2.5 hours. FIGS. 15A-15H show density plots of PI (channel 3; FL3-A) for: cell only control (FIG. 15A); cells loaded with DHR 123 only (FIG. 15B); cells treated with DHR 123 and PMA (FIG. 15C); cells treated with DHR 123 and ragweed pollen (2.5 μL/100 μL blood) (FIG. 15D); cells treated with DHR 123 and ragweed pollen (5 μL/100 μL blood) (FIG. 15E), cells treated with DHR 123 and milk (5 μL/100 μL blood) (FIG. 15F); cells treated with cucumber (5 μL/100 μL blood) (FIG. 15G), and cells treated with basil (5 μL/100 μL blood) (FIG. 15H). All groups were incubated with or without stimulant for 2.5 hours.

FIGS. 16A-16H depict histogram plots of DHR 123 and PI flow cytometry blood testing following exposure to ragweed pollen, milk, cucumber, and basil stimulants for 2.5 hours using PI (channel 3, FL3-A). FIGS. 16A-16H show histogram plots of PI (channel 3; FL3-A) for: cell only control (FIG. 16A); cells loaded with DHR 123 only (FIG. 16B); cells treated with DHR 123 and PMA (FIG. 16C); cells treated with DHR 123 and ragweed pollen (2.5 μL/100 μL blood) (FIG. 16D); cells treated with DHR 123 and ragweed pollen (5 μL/100 L blood) (FIG. 16E), cells treated with DHR 123 and milk (5 μL/100 μL blood) (FIG. 16F); cells treated with DHR 123 and cucumber (5 μL/100 μL blood) (FIG. 16G), and cells treated with basil (5 μL/100 μL blood) (FIG. 16H). All groups were incubated with or without stimulant for 2.5 hours.

FIGS. 17A-17H depict density and histogram plots of DHR 123 and PI flow cytometry blood testing following exposure to dust mite and ragweed pollen stimulants for 2.5 hours using RH 123 (Channel 1; FL1-A). FIG. 17A and FIG. 17E show density and histogram plots, respectively, of the group of cells loaded with DHR 123 only. FIG. 17B and FIG. 17F show density and histogram plots, respectively, of the group of cells loaded with DHR 123 and treated with PMA. FIG. 17C and FIG. 17G show density and histogram plots, respectively, of the group of cells loaded with DHR 123 and dust mite solution (8 μL/100 μL blood). FIG. 17D and FIG. 17H show density and histogram plots, respectively, of the group of cells loaded with DHR 123 and ragweed pollen solution (8 μL/100 μL blood).

FIGS. 18A-18H depict density and histogram plots of DHR 123 and PI flow cytometry blood testing following exposure to dust mite and ragweed pollen stimulants for 2.5 hours using PI (Channel 3; FL3-A). FIG. 18A and FIG. 18E show density and histogram plots, respectively, of the group of cells loaded with DHR 123 only. FIG. 18B and FIG. 18F show density and histogram plots, respectively, of the group of cells loaded with DHR 123 and treated with PMA. FIG. 18C and FIG. 18G show density and histogram plots, respectively, of the group of cells loaded with DHR 123 and dust mite solution (8 μL/100 μL blood). FIG. 18D and FIG. 18H show density and histogram plots, respectively, of the group of cells loaded with DHR 123 and ragweed pollen solution (8 μL/100 μL blood).

FIGS. 19A-19L depict density plots of DHR 123 flow cytometry blood testing following exposure to dust mite stimulant for 1.5 hours using RH 123, CD16, CD193 and CD123. FIGS. 19A-19L show density plots of RH 123 (channel 1; FL1-A), CD16 (channel 2; FL2-A), CD193 (channel 3, FL3-A), and CD123 (channel 4, FL4-A) for the DHR only control group (FIGS. 19A-19D, respectively), DHR 123 and PMA treated group (FIGS. 19E-19H, respectively), and DHR 123 and dust mite treated group (FIGS. 19I-19L, respectively). All groups were incubated with or without stimulant for 1.5 hours.

FIGS. 20A-20C depict histograms of DHR 123flow cytometry blood testing following exposure to dust mite stimulant for 1.5 hours using CD16 (Channel 2; FL2-A). FIGS. 20A-20C show histograms and mean fluorescent intensity (MFI) of CD16 surface marker (channel 2; FL2-A) for the DHR 123 only control group (FIG. 20A, MFI=27956), DHR 123 and PMA treated group (FIG. 20B, MFI=11120), and DHR 123 and dust mite treated group (FIG. 20C, MFI=16358). All groups were incubated with or without stimulant for 1.5 hours.

FIGS. 21A-21L depict density plots of DHR 123 flow cytometry blood testing following exposure to dust mite and ragweed pollen stimulants for 2.5 hours using RH 123, CD16, CD193 and CD123. FIGS. 21A-21L show density plots of RH 123 (channel 1; FL1-A), CD16 (channel 2; FL2-A), CD193 (channel 3, FL3-A), and CD123 (channel 4, FL4-A) for the DHR 123 only treated control group (FIGS. 21A-21D), DHR 123 and dust mite treated group (FIGS. 21E-21H), and DHR 123 and ragweed pollen treated group (FIGS. 21I-21L). All groups were incubated with or without stimulant for 2.5 hours.

FIGS. 22A-22C depict histograms of DHR 123 flow cytometry blood testing following exposure to dust mite and ragweed stimulants for 2.5 hours using CD16. FIGS. 22A-22C show histograms and mean fluorescent intensity (MFI) of CD16 surface marker (channel 2; FL2-A) for the DHR 123 only treated control group (FIG. 22A, MFI=11677), DHR 123 and dust mite treated group (FIG. 22B, MFI=3908), and DHR 123 and ragweed pollen treated group (FIG. 22C, MFI=2713). All groups were incubated with or without stimulant for 2.5 hours.

FIGS. 23A-23F depict density plots of CD193-gated DHR 123 flow cytometry blood testing following exposure to dust mite stimulant for 1.5 hours. FIGS. 23A-23C show density plots of CD193 signals (channel 3; FL3-A for the DHR 123 only treated control group (FIG. 23A), DHR 123 and PMA treated group (FIG. 23B), and DHR 123 and dust mite treated group (FIG. 23C). FIGS. 23D-23F show the gated RH 123 signals and mean fluorescent intensity corresponding to each group (MFI=32944, MFI=70320, and MFI=28522, respectively). All groups were incubated with or without stimulant for 1.5 hours.

FIGS. 24A-24F depict density plots of CD193 gated DHR 123 flow cytometry blood testing following exposure to dust mite and ragweed pollen stimulants for 2.5 hours. FIGS. 24A-24C show density plots of CD193 signals (channel 3; FL3-A) for the DHR 123 only treated control group (FIG. 24A), DHR 123 and dust mite treated group (FIG. 24B), and DHR 123 and ragweed pollen treated group (FIG. 24C). FIGS. 24D-24F show the gated RH 123 signals and mean fluorescent intensity corresponding to each group (MFI=10156, MFI=19911, and MFI=17210, respectively). All groups were incubated with or without stimulant for 2.5 hours.

FIGS. 25A-25F depict density plots of CD123 gated DHR 123 flow cytometry blood testing following exposure to dust mite stimulant for 1.5 hours. FIGS. 25A-25C show density plots of CD123 signals (channel 4; FL4-A for the DHR 123 only treated control group (FIG. 25A), DHR 123 and PMA treated group (FIG. 25B), and DHR 123 and dust mite treated group (FIG. 25C). FIGS. 25D-25F show the gated RH 123 signals and mean fluorescent intensity corresponding to each group (MFI=2055, MFI=6388, and MFI=3023, respectively). All groups were incubated with or without stimulant for 1.5 hours.

FIGS. 26A-26F depict density plots of CD123 gated DHR 123 flow cytometry blood testing following exposure to dust mite and ragweed pollen stimulants for 2.5 hours. FIGS. 26A-26C show density plots of CD123 signals (channel 4; FL4-A for the DHR 123 only treated control group (FIG. 26A), DHR 123 and dust mite treated group (FIG. 26B), and DHR 123 and ragweed pollen treated group (FIG. 26C). FIGS. 26D-26F show the gated RH 123 signals and mean fluorescent intensity corresponding to each group (MFI=1800, MFI=3299, and MFI=1270, respectively). All groups were incubated with or without stimulant for 2.5 hours.

FIGS. 27A-27B depict histogram and density plot of compensation beads with primary (mouse anti-human citrullinated histone H3) and secondary antibodies (goat anti-mouse APC conjugated) for DHR 123 flow cytometry blood testing. RH 123 is channel 1 (FL1-A), PI is channel 3 (FL3-A) and citrullinated histone H3 is channel 4 (FL4-A).

FIGS. 28A-28L depict density plots of channel FSC-A, RH 123 (channel 1, FL1-A), PI (channel 3, FL3-A), and citrullinated histone H3 (channel 4, FL4-A) vs. SSC-A. FIGS. 28A-28D show cell-only control group; FIGS. 28E-28H show DHR 123 and PMA treated group, FIGS. 28I-28L show DHR 123 and dust mite stimulant-exposed group.

FIGS. 29A-29C depict gated density plots of PI (channel 3, FL3-A) vs. citrullinated histone H3 (channel 4, FL4-A). FIG. 29A shows cell-only control group; FIG. 29B shows DHR 123 and PMA treated group; FIG. 29C shows DHR 123 and dust mite stimulant-exposed group.

FIGS. 30A-30P depict density plots of channel FSC-A, RH 123 (channel 1, FL1-A), PI (channel 3, FL3-A), and citrullinated histone H3 (channel 4, FL4-A) vs. SSC-A. FIGS. 30A-30D show cell-only control group; FIGS. 30E-30H show DHR 123 and PMA treated group, FIGS. 30I-30L show DHR 123 and dust mite stimulant-exposed group; FIGS. 30M-30P show ragweed pollen stimulant-exposed group.

FIGS. 31A-31D depict gated density plots of PI (channel 3, FL3-A) vs. citrullinated histone H3 (channel 4, FL4-A). FIG. 31A shows DHR 123 only treated group;

FIG. 31B shows DHR 123 and PMA treated group; FIG. 31C shows DHR 123 and dust mite stimulant-exposed group; FIG. 31D shows DHR 123 and ragweed pollen stimulant-exposed group.

FIGS. 32A-32L depict activation of human granulocytes following exposure to vaccine component medicament stimulants as assessed by DHR 123 flow cytometry blood testing. FIGS. 31A-31C show control density plots. FIGS. 31D-31F show density plots of DHR 123 and (4-hydroxybutyl) azanediyl)bis(hexane-6,1-diyl)bis(2-hexyldecanoate)] (ALC-0315) stimulant treated group. FIGS. 31G-31I show density plots of DHR 123 and 2,2-dimyristoyl-rac-glycero3-methoxypolyethylene glycol-2000 (PEG2000-DMG) stimulant treated group. FIGS. 31J-31L show density plots of DHR 123 and 2-[(polyethylene glycol)-2000]-N,N-ditetradecylacetamide (ALC-0159) stimulant treated group.

FIGS. 33A-33C depict basophil activation following exposure of human blood sample to components of COVID-19 vaccines. FIG. 33A shows activated CD63+/CD203c+/CD193− and CD63+/CD203c+/CD193+ basophil reactivity to components ALC0315, ALC0159, PEG2000-DMG, and DSPC components of the BNT162b2 COVID-19 vaccine (Pfizer). FIG. 33B shows activated CD63+/CD203c+/CD193− and CD63+/CD203c+/CD193+ basophil reactivity to components DSPC, SM102, and PEG2000-DMG components of the mRNA-1273 COVID-19 vaccine (Moderna). FIG. 33C shows activated CD63+/CD203c+/CD193− and CD63+/CD203c+/CD193+ basophil reactivity to components EtOH, HBCD, and PS80 components of the Ad26.COV2.S COVID-19 vaccine (Johnson and Johnson).

FIGS. 34A-34B depict basophil and neutrophil activation following exposure of human blood sample to COVID-19 vaccines. FIG. 34A shows basophil activation (% CD63+/CD203c+) for BNT162b2 (Pfizer), mRNA-1273 (Moderna), and Ad.COV2.S (Johnson & Johnson) vaccines. FIG. 34B shows neutrophil activation (% CD11b+) for BNT162b2, mRNA-1273, and Ad.COV2.S vaccines.

FIGS. 35A-35C depict basophil and neutrophil activation following exposure of human blood sample to components of COVID-19 vaccines. FIG. 35A shows neutrophil activation (CD11b+) to components of the BNT162b2 vaccine (Pfizer). FIG. 35B shows neutrophil activation (CD11b+) to components of the mRNA-1273 vaccine (Moderna). FIG. 35C shows neutrophil activation (CD11b+) to components of the Ad.COV2.S vaccine (Johnson & Johnson).

### 4. DETAILED DESCRIPTION

### 4.1 Definitions

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the methods provided herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

It should also be understood that the terms “about,” “approximately,” “generally,” “substantially,” and like terms, used herein when referring to a dimension or characteristic of a component of the methods provided herein, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The term “antigen” refers to any molecule (e.g., protein, peptide, polysaccharide, glycoprotein, glycolipid, nucleic acid, portions thereof, or combinations thereof) that is capable of mediating an immune response. Exemplary immune responses include antibody production and activation of immune cells, such as T cells, B cells or NK cells.

The terms “antigen binding fragment” or “antigen binding domain” refers to a portion of a protein that binds the antigen. Antigen binding fragments may be synthetic, enzymatically obtainable or genetically engineered polypeptides and include portions of an immunoglobulin that bind an antigen, such as a VH, a VL, the VH and the VL, Fab, Fab′, F(ab′)₂, Fd and Fv fragments, domain antibodies (dAb) consisting of one VH domain or one VL domain, camelized VH domains, VHH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3, alternative scaffolds that bind an antigen, and multispecific proteins comprising the antigen binding fragments. Antigen binding fragments (such as the VH and the VL) may be linked together via a synthetic linker to form various types of single antibody designs in which the VH/VL domains may pair intramolecularly, or intermolecularly in those cases when the VH and the VL domains are expressed by separate single chains, to form a monovalent antigen binding domain, such as single chain Fv (scFv) or diabody. Antigen binding fragments may also be conjugated to other antibodies, proteins, antigen binding fragments or alternative scaffolds which may be monospecific or multispecific to engineer bispecific and multispecific proteins.

The term “antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific, etc., dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g., IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4. Immunoglobulins may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

The term “complementarity determining regions” (CDRs) refers to antibody regions that bind an antigen. There are three CDRs in the VH (HCDR1, HCDR2, HCDR3) and three CDRs in the VL (LCDR1, LCDR2, LCDR3). CDRs may be defined using various delineations such as Kabat (Wu et al., (1970) J Exp Med 123: 211-250); Kabat et al., Sequences of Proteins of Immunological Interest, 5th Ed. Public Health Service, National Institutes of Health, Bethesda, Md., 1991), Chothia (Chothia et al., (1987) J Mol Biol 196:901-17), IMGT (Lefranc et al., (2003) Dev Comp Immunol 27: 55-77) and AbM (Martin and Thornton (1996) J Bmol Biol 263: 800-815). The correspondence between the various delineations and variable region numbering is described (see e.g., Lefranc et al., (2003) Dev Comp Immunol 27: 55-77; Honegger and Pluckthun, J Mol Biol (2001) 309:657-670; International ImMunoGeneTics (IMGT) database; Web resources, http://www_imgt_org). Available programs such as abYsis by UCL Business PLC may be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated.

The terms “decrease,” “lower” or “reduce,” refer generally to the ability of a test molecule to mediate a reduced response (i.e., downstream effect) when compared to the response mediated by a control or a vehicle. Decrease may be a statistically significant difference in the measured response between the test molecule and the control (or the vehicle), or a decrease in the measured response, such as a decrease of about 1.1, 1.2, 1.5, 2, 3, 4, 5, 6, 7, 8, 9, 10, 15, 20 or 30 fold or more, such as 500, 600, 700, 800, 900 or 1000 fold or more.

The terms “enhance,” “promote” or “increase,” refer generally to the ability of the test molecule to mediate a greater response (i.e., downstream effect) when compared to the response mediated by a control or a vehicle. Enhance may be a statistically significant difference in the measured response between the test molecule and control (or vehicle), or an increase in the measured response, such as an increase of about 1.1, 1.2, 1.5, 2, 3, 4, 5, 6, 7, 8, 9, 10, 15, 20 or 30 fold or more, such as 500, 600, 700, 800, 900 or 1000 fold or more.

The term “monoclonal antibody” refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation. Monoclonal antibodies typically bind one antigenic epitope. A bispecific monoclonal antibody binds two distinct antigenic epitopes. Monoclonal antibodies may have heterogeneous glycosylation within the antibody population. Monoclonal antibody may be monospecific or multispecific such as bispecific, monovalent, bivalent or multivalent.

The term “multispecific” refers to a molecule that binds two or more distinct antigens or two or more distinct epitopes within the same antigen. Multispecific molecule may have cross-reactivity to other related antigens, for example to the same antigen from other species (homologs), such as human or monkey, for example Macaca fascicularis (cynomolgus, cyno) or Pan troglodytes, or may bind an epitope that is shared between two or more distinct antigens.

The term “recombinant” refers to polynucleotides, polypeptides, vectors, viruses and other macromolecules that are prepared, expressed, created or isolated by recombinant means.

The terms “single chain Fv” or “scFv” refer to a single chain protein comprising a VH, a VL and a linker between the VH and the VL. The scFv may have the VL and VH variable regions in either orientation, e.g., with respect to the N- to C-terminal order of the VH and the VL. The scFv may thus be in the orientation VL-linker-VH or VH-linker-VL. scFv may be engineered to comprise disulfide bonds between the VH, the VL and the linker.

The terms “specifically binds,” “specific binding,” “specifically binding” or “binds” refer to a protein such as a scFv binding to an antigen or an epitope within the antigen with greater affinity than for other antigens. Typically, the protein, such as the scFv binds to the antigen or the epitope within the antigen with an equilibrium dissociation constant (K_{D}) of about 1×10⁻⁶ M or less, about 1×10⁻⁷ M or less, about 5×10⁻⁸ M or less, about 1×10⁻⁸ M or less, about 1×10⁻⁹ M or less, about 1×10⁻¹⁰ M or less, about 1×10⁻¹¹ M or less, or about 1×10⁻¹² M or less, typically with the K_{D} that is at least one hundred fold less than its K_{D} for binding to a non-specific antigen (e.g., BSA, casein).

The term “subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” can be used interchangeably herein.

“Treat,” “treating” or “treatment” of a disease or disorder refers to accomplishing one or more of the following: reducing the severity and/or duration of the disorder, inhibiting worsening of symptoms characteristic of the disorder being treated, limiting or preventing recurrence of the disorder in subjects that have previously had the disorder, or limiting or preventing recurrence of symptoms in subjects that were previously symptomatic for the disorder.

### 4.2 Methods

In one aspect, provided herein is a method for detecting or measuring a response of a subject to an exogenous stimulant. In another aspect, provided is a method for determining or predicting an immune reaction of a subject to an exogenous stimulant. In some embodiments, said predicted immune reaction of the subject to the exogenous stimulant is immune reaction type and/or immune reaction severity. In another aspect, provided is a method for determining or predicting the compatibility of a subject with an exogenous stimulant.

In certain embodiments, said methods comprise the use of fluorometric measurement on a biological sample of a subject to determine the response of the subject to exogenous stimulant. In some embodiments, a method as described herein comprises treating or contacting a biological sample with an oxidation-sensitive fluorophore. In some embodiments, the biological sample may be enriched for granulocytes. In some embodiments, the biological sample is not enriched for granulocytes. In some embodiments, the biological sample is freshly drawn peripheral blood. In some embodiments, the oxidation-sensitive fluorophore is oxidized by granulocytes present in the biological sample. In some embodiments, a method as described herein comprises treating or contacting granulocytes in a biological sample with an oxidation-sensitive fluorophore.

In certain embodiments of the methods, granulocytes of a biological sample produce reactive oxygen species (ROS) following contact with an exogenous stimulant. In some embodiments, a biological sample comprises a higher amount of ROS following contact with an exogenous stimulant compared to a biological sample that is not contacted with the exogenous stimulant. In some embodiments, the reference exogenous stimulant is selected from Section 4.6.

In certain embodiments, a method as described herein comprises measuring an increase in fluorescence following oxidation of the oxidation-sensitive fluorophore. In some embodiments, a method as described herein comprises measuring a decrease in fluorescence following oxidation of the oxidation-sensitive fluorophore. In some embodiments, a method as described herein comprises measuring substantially unchanged fluorescence following oxidation of the oxidation-sensitive fluorophore. In certain embodiments, the oxidation-sensitive fluorophore is oxidized by reactive oxygen species (ROS) released by granulocytes of biological sample following contact with an exogenous stimulant. In some embodiments, a method as described herein comprises measuring the amount of oxidation-sensitive fluorophore-treated granulocytes in a biological sample from the subject. In some embodiments, a method as described herein comprises measuring the amount of activated granulocytes in the biological sample following contact of the granulocytes with the exogenous stimulant. In some embodiments, contact of the granulocytes of the biological sample with exogenous stimulant is performed ex vivo. In some embodiments, contact of the granulocytes of the biological sample with exogenous stimulant is performed in vivo.

In certain embodiments, the method further comprises contacting the oxidation-sensitive fluorophore fluorophore-treated granulocytes with one or more binding moieties that bind to a basophil, eosinophil, or neutrophil, and wherein the one or more binding moieties are detectable by flow cytometry.

In certain embodiments, the method further comprises determination of granulocyte viability. In some embodiments, determination of granulocyte viability is by cell staining. In some embodiments, the granulocytes are contacted with a nuclear stain and/or DNA-intercalating stain. In some embodiments, the granulocytes are contacted with propidium iodide (PI) stain.

In certain embodiments, the method further comprises determination of positive extracellular trap formation. In some embodiments, the granulocytes are contacted with a nuclear stain and/or DNA-intercalating stain. In some embodiments, the granulocytes are contacted with propidium iodide (PI) stain. In some embodiments, the granulocytes are contacted with an anti-citrullinated histone H3 (H3cit) antibody.

In certain embodiments, a method for detecting or measuring the response of a subject to an exogenous stimulant as described herein comprises the use of fluorometric measurement of the amount of oxidation-sensitive fluorophore-treated granulocytes in a biological sample from the subject after contact of the granulocytes with the exogenous stimulant ex vivo, wherein the method further comprises contacting the oxidation-sensitive fluorophore-treated granulocytes with one or more binding moieties that bind to a basophil, eosinophil, or neutrophil, and wherein the one or more binding moieties are detectable by flow cytometry, and wherein an increase in intensity of fluorescence from an oxidized form of the oxidation-sensitive fluorophore as measured by flow cytometry after contact of the oxidation-sensitive fluorophore-treated granulocytes with the exogenous stimulant as compared to a reference control indicates a response of the subject to the exogenous stimulant.

In certain embodiments, a method for determining or predicting an immune reaction of a subject to an exogenous stimulant as described herein comprises the use of fluorometric measurement of the amount of oxidation-sensitive fluorophore-treated granulocytes in a biological sample from the subject after contact of the granulocytes with the exogenous stimulant ex vivo, wherein the method further comprises contacting the oxidation-sensitive fluorophore-treated granulocytes with one or more binding moieties that bind to a basophil, eosinophil, or neutrophil, and wherein the one or more binding moieties are detectable by flow cytometry, and wherein an increase in intensity of fluorescence from an oxidized form of the oxidation-sensitive fluorophore as measured by flow cytometry after contact of the oxidation-sensitive fluorophore-treated granulocytes with the exogenous stimulant as compared to a reference control indicates an immune reaction of the subject to the exogenous stimulant. In certain embodiments, the predicted immune reaction of the subject to the exogenous stimulant is immune reaction type and/or immune reaction severity.

In certain embodiments, a method for determining or predicting the compatibility of a subject with an exogenous stimulant as described herein comprises the use of fluorometric measurement of the amount of oxidation-sensitive fluorophore-treated granulocytes in a biological sample from the subject after contact of the granulocytes with the exogenous stimulant ex vivo, wherein the method further comprises contacting the oxidation-sensitive fluorophore-treated granulocytes with one or more binding moieties that bind to a basophil, eosinophil, or neutrophil, and wherein the one or more binding moieties are detectable by flow cytometry, and wherein a decrease in or substantially unchanged intensity of fluorescence from an oxidized form of the oxidation-sensitive fluorophore as measured by flow cytometry after contact of the oxidation-sensitive fluorophore-treated granulocytes with the exogenous stimulant as compared to a reference control indicates compatibility of the subject with the exogenous stimulant.

In certain aspects, provided is a method of predicting or identifying an immune reaction in a subject to an exogenous stimulant, comprising:
(i) obtaining a biological sample from the subject;
(ii) isolating a population of leukocytes from the blood sample of (i);
(iii) diffusing into the population of leukocytes of (ii) an amount of oxidation-sensitive fluorophore, and contacting the population with the exogenous stimulant, and one or more binding moieties selected from:
   a) an anti-neutrophil binding moiety that binds to a neutrophil cell surface marker;
   b) an anti-eosinophil binding moiety that binds to a eosinophil cell surface marker; or
   c) an anti-basophil binding moiety that binds to a basophil cell surface marker,
   wherein the one or more binding moieties are detectable by flow cytometry;
(iv) isolating via flow cytometry one or more populations of neutrophils, eosinophils, and/or basophils from the leukocytes of (iii) based on cell surface marker positivity or negativity and/or side scattering profile;
(v) measuring via fluorometric measurement the level of an oxidized form of an oxidation-sensitive fluorophore in the one or more populations of (iv);
(vi) comparing the level of an oxidized form of an oxidation-sensitive fluorophore of (v) with a level of an oxidized form of an oxidation-sensitive fluorophore of a reference control as measured via fluorometric measurement, wherein the reference control is prepared from the same or substantially the same one or more populations of (iv) as in (v); and
(vii) identifying the one or more populations of cells in (vi) having higher level of the oxidized form of the oxidation-sensitive fluorophore compared to the level of the oxidized form of the oxidation-sensitive fluorophore of the reference control.

In certain aspects, provided herein is a method for detecting the activation of a neutrophil, eosinophil, or basophil cell population in a biological sample of a subject following exposure to an exogenous stimulant, comprising:
(i) contacting biological sample with the exogenous stimulant and an oxidation-sensitive fluorophore;
(ii) detecting via flow cytometry the cell surface expression of one or more neutrophil, eosinophil, or basophil cell surface markers in the leukocyte population of the biological sample;
(iii) detecting degranulation of said neutrophil, eosinophil, or basophil population by detecting oxidation of an oxidation-sensitive fluorophore into an oxidized form of said fluorophore; and
(iv) concluding that the neutrophil, eosinophil, or basophil population that oxidizes the oxidation-sensitive fluorophore into the oxidized form are activated.

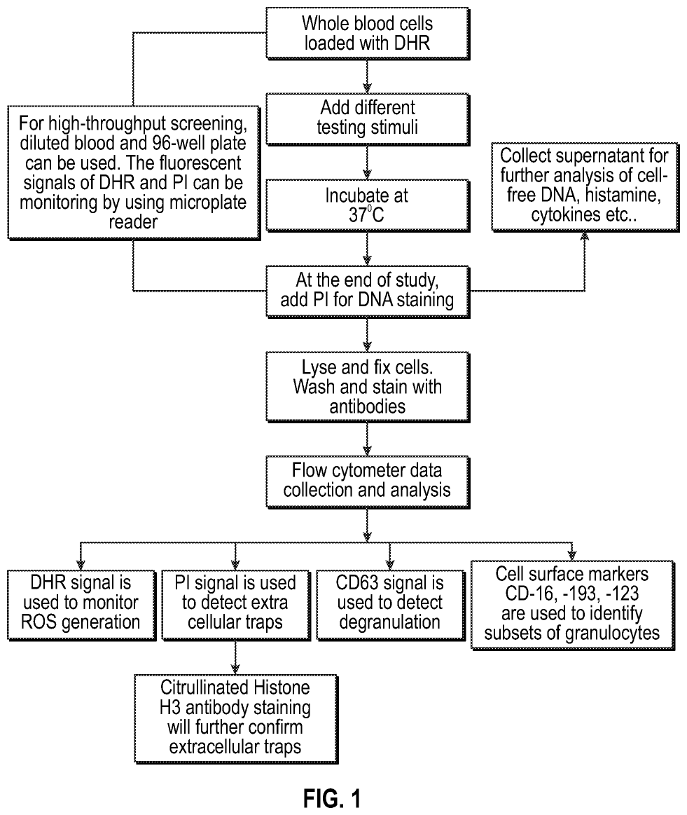

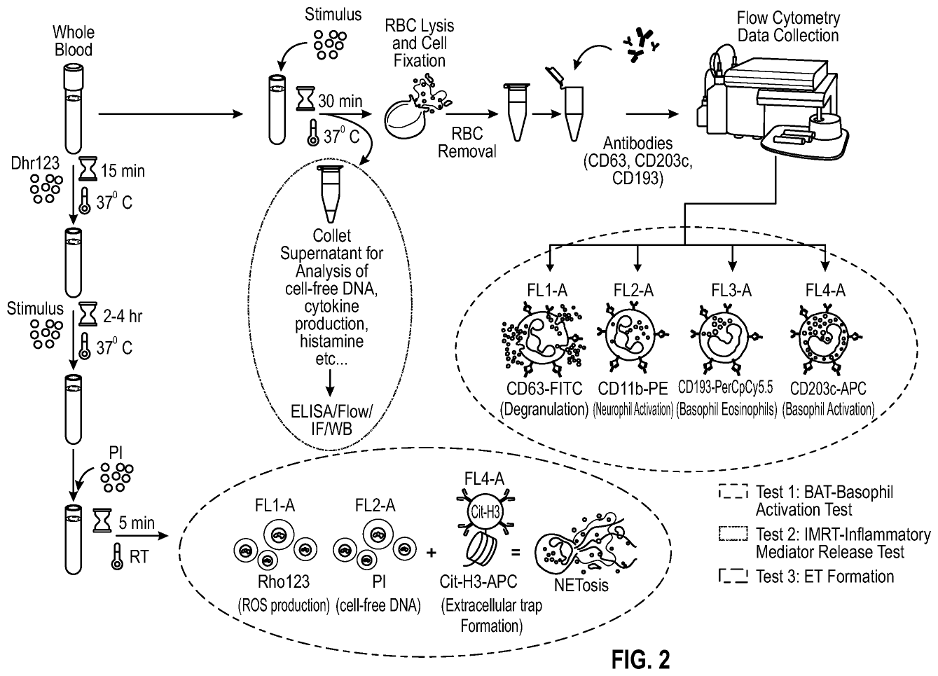

In certain embodiments, the method is as described in FIG. 1. In certain embodiments, the method is as described in FIG. 2.

In certain embodiments of a method as described herein, the red blood cells in the biological sample are lysed after contact with the exogenous stimulant. In certain embodiments, the granulocytes in the biological sample are fixed after contact with the exogenous stimulant. In certain embodiments, a method as described herein is completed in about 3 hours or less, about 2.5 hours or less, about 2.0 hours or less, about 1.5 hours or less, about 1.0 hours or less, or about 0.5 hours or less.

In specific embodiments, fluorometric measurement is by flow cytometry or plate reader. In specific embodiments, the biological sample is a blood sample. In specific embodiments, the cell surface marker is a neutrophil cell surface marker, eosinophil cell surface marker, and/or basophil cell surface marker. In specific embodiments, the cell surface marker is selected from CD11b, CD16, CD33, CD63, CD123, CD193, or CD203c. In specific embodiments, the granulocyte and/or activated granulocyte is selected from a basophil, eosinophil, or neutrophil. In specific embodiments, the antibody detectable by flow cytometry specifically binds to a basophil, eosinophil, or neutrophil. In specific embodiments, the oxidation-sensitive fluorophore is DHR 123.

In certain embodiments, a method as described herein comprises comparing the intensity of fluorescence with a reference control. In certain embodiments, the reference control is oxidation-sensitive fluorophore-treated granulocytes in a biological sample from the subject before contact of the granulocytes with the exogenous stimulant ex vivo. In certain embodiments, the reference control is oxidation-sensitive fluorophore-treated granulocytes in a biological sample from the subject that is not contacted with the exogenous stimulant ex vivo. In certain embodiments, the reference control is oxidation-sensitive fluorophore-treated granulocytes in a biological sample from the subject treated with an stimulant standard ex vivo. In some embodiments, the stimulant standard is an exogenous stimulant that produces linear or logarithmically linear dose-dependent fluorescence when combined with an oxidation-sensitive fluorophore and ROS-producing granulocytes. In some embodiments, the stimulant standard is an exogenous stimulant that produces linear or logarithmically linear dose-dependent immune response in a subject.

In certain embodiments, comparison to a reference control is by quantified fluorescence after exposure of the biological sample to an exogenous stimulant. A biological sample containing granulocytes may upon treatment with an oxidation-sensitive fluorophore and exposure to an exogenous stimulant show increased, decreased, or substantially unchanged intensity of fluorescence in comparison to a reference control. In certain embodiments, intensity of fluorescence is increased by at least about 10%, at least about 20%, at least about 30%, at least about 40%, at least about 50%, at least about 60%, at least about 70%, at least about 80%, at least about 90%, at least about 2-fold, at least about 3-fold, at least about 4-fold, or at least about 5-fold in the biological sample as compared to the reference control. In some embodiments, an increase in intensity of fluorescence as compared to the reference control indicates a positive immune reaction in the subject to the exogenous stimulant. In certain embodiments, intensity of fluorescence is not increased or decreased by about 10% or less in the biological sample as compared to the reference control. In certain embodiments, intensity of fluorescence is substantially unchanged in the biological sample as compared to the reference control. In some embodiments, a substantially unchanged intensity of fluorescence as compared to the reference control indicates a positive immune reaction in the subject to the exogenous stimulant. In some embodiments, a substantially unchanged intensity of fluorescence as compared to the reference control indicates a negative immune reaction in the subject to the exogenous stimulant. In certain embodiments, intensity of fluorescence is decreased by at least about 10%, at least about 20%, at least about 30%, at least about 40%, at least about 50%, at least about 60%, at least about 70%, at least about 80%, at least about 90%, at least about 2-fold, at least about 3-fold, at least about 4-fold, or at least about 5-fold in the biological sample as compared to the reference control. In some embodiments, a decrease in intensity of fluorescence as compared to the reference control indicates an absence of or negative immune reaction in the subject to the exogenous stimulant.

In another aspect, provided is a use of any one of the methods as described herein ahead of or for the treatment of a disease or disorder in the subject. For example, a standard therapy for treating a particular disease or disorder in the subject may be modified by determining or predicting by a method as described herein that the subject has or would have an immune reaction (e.g., allergic reaction) to one or more medicament components of the standard therapy. Such an approach is useful in determining the compatibility of subject with a particular medicament (e.g., components of a vaccine) prior to or concurrently with initiating standard therapy with said medicament.

### 4.3 Oxidation-Sensitive Fluorophores

In certain embodiments of a method as described herein, the oxidation-sensitive fluorophore is selected from dihydrorhodamine (e.g., dihydrorhodamine 123 (DHR 123)), dihydroethidine (DHE), dichlorodihydrofluorscein (DCFH2), or a reduced fluorescein derivative.

In certain embodiments, the oxidized form of the oxidation-sensitive fluorophore is selected from rhodamine (e.g., rhodamine 123 (RH 123)), 2-hydroxyethidium, dichlorofluorescein, or an oxidized fluorescein derivative.

The some embodiments, the fluorophore is an oxidation-sensitive form of fluorescein, or its derivatives, such as an oxidation sensitive fluorescein-5-isothiocyanate (FITC), 5-(and 6)-carboxyfluorescein, 5- or 6-carboxyfluorescein, 6-(fluorescein)-5-(and 6)-carboxamido hexanoic acid, fluorescein isothiocyanate; rhodamine, or its derivatives, such as tetramethylrhodamine and tetramethylrhodamine-5-(and-6)-isothiocyanate (TRITC). In certain embodiments, the fluorophore may comprise an oxidation-sensitive coumarin dye, such as oxidation-sensitive (diethyl-amino)coumarin or 7-amino-4-methylcoumarin-3-acetic acid, succinimidyl ester (AMCA); sulforhodamine 101 sulfonyl chloride, TexasRed™ TexasRed™ sulfonyl chloride; 5-(and-6)-carboxyrhodamine 101, succinimidyl ester, also known as 5-(and-6)-carboxy-X-rhodamine, succinimidyl ester (CXR); lissamine or lissamine derivatives such as lissamine rhodamine B sulfonyl Chloride (LisR); 5-(and-6)-carboxyfluorescein, succinimidyl ester (CFI); fluorescein-5-isothiocyanate (FITC); 7-diethylaminocoumarin-3-carboxylic acid, succinimidyl ester (DECCA); 5-(and-6)-carboxytetramethyirhodamine, succinimidyl ester (CTMR); 7-hydroxycoumarin-3-carboxylic acid, succinimidyl ester (HCCA); 6-fluorescein-5-(and-6)-carboxamidolhexanoic acid (FCHA); N-(4,4-difluoro-5,7-dimethyl-4-bora-3a,4a-diaza-3-indacenepropionic acid, succinimidyl ester; also known as 5,7-dimethyl BODIPY™ propionic acid, succinimidyl ester (DMBP); “activated fluorescein derivative” (FAP), available from Molecular Probes, Inc.; eosin-5-isothiocyanate (EITC); 24rythrosine-5-isothiocyanate (ErITC); and Cascade™ Blue acetylazide (CBAA) (the O-acetylazide derivative of 1-hydroxy-3,6,8-pyrenetrisulfonic acid). In certain embodiments, the oxidation-sensitive fluorophore comprises a fluorescent protein such as phycoerythrin, allophycocyanin, green fluorescent protein and its analogs or derivatives, fluorescent amino acids such as tyrosine and tryptophan and their analogs, fluorescent nucleosides, or other fluorescent molecules such as organic dyes, including Cy2, Cy3, Cy 3.5, Cy5, Cy5.5, Cy 7, IR dyes, Dyomics dyes, Oregon green 488, pacific blue, rhodamine green, and Alexa dyes. In certain embodiments, the oxidation-sensitive fluorophore may take advantage of fluorescence energy transfer and comprise conjugates of R-phycoerythrin or allophycocyanin to organic dyes, such as Cy2, Cy3, Cy 3.5, Cy5, Cy5.5, Cy 7, Dyomics dyes, or Alexa dyes. In certain embodiments, the oxidation-sensitive fluorophore may comprise an inorganic fluorescent colloidal particle such as a quantum dot or other fluorescent nanoparticle, such as particles based on semiconductor material like CdS-coated CdSe nanocrystallites.

In certain embodiments of a method as described herein, the granulocytes are treated with from about 0.1 μg/mL to about 50 μg/mL of oxidation-sensitive fluorophore. In some embodiments, the granulocytes are treated with from about 1 μg/mL to about 5 μg/mL of oxidation-sensitive fluorophore. In some embodiments, the granulocytes are treated with about 0.5 μg/mL, about 1 μg/mL, about 1.5 μg/mL, about 2 μg/mL, about 2.5 μg/mL, about 3 μg/mL, about 3.5 μg/mL, about 4 μg/mL, about 4.5 μg/mL, or about 5 μg/mL of oxidation-sensitive fluorophore.

### 4.4 Fluorometric Measurement

Fluorescence generally refers to the physical process in which light is emitted from the compound after a short interval following absorption of radiation. Generally, the emitted light is of lower energy and longer wavelength than that absorbed. In certain embodiments, the energy may be transferred from one fluorophore to another prior to emission of light. In certain embodiments, the fluorescence of the fluorophores used herein can be detected using standard techniques to measure fluorescence.

In certain embodiments of a method as described herein, fluorometric measurement is performed via singleplex or multiplex immunodetection assay. In some embodiments, the fluorometric measurement performed via flow cytometry, plate reader, microscopy, imaging, immunohistochemistry (IHC), high content screening (HCS), immunocytochemistry (ICC), immunomagnetic cellular depletion, immunomagnetic cell capture, in situ hybridization (ISH), enzyme immune-assay (EIA), enzyme-linked immune-assay (ELISA), ELISpot, arrays including bead arrays, multiplex bead array, microarray, antibody array, cellular array, solution phase capture, chemiluminescence detection, infrared detection, blotting method, a Western blot, a Southern blot, a Southwestern blot, labeling inside an electrophoresis system, labeling on a surface, labeling on an array, PCR amplification, elongation followed by PCR amplification, immunoprecipitation, co-immunoprecipitation, chromatin immunoprecipitation, pretargeting imaging, therapeutic agent, or combinations thereof. In specific embodiments, fluorometric measurement is performed via flow cytometry or plate reader. In specific embodiments, fluorometric measurement is performed via flow cytometry in combination with side scatter analysis. A suitable method of fluorescent measurement may comprise a multiplex assay, utilizing one or more fluorescent probes.

A limiting feature for success of flow cytometry analysis to detect antigens present in or on individual cells is the sensitivity and specificity of detection of that antigen. In general, antibodies are commonly used as probes, given their properties as sensitive and specific detection reagents. When the antibodies are rendered fluorescent, they may be detected by flow cytometry. Direct fluorescent labeling of antibodies to form stable, covalent antibody-fluorophore conjugates allows their facile use in flow cytometry, but may alter the favorable properties of the antibody as a detection reagent. In particular, conjugation to multiple small organic fluorophores may inactivate a significant fraction of antibodies or alter solubility. An antibody-oligonucleotide conjugate may be used for flow cytometry as an alternative to an antibody-fluorophore conjugate.

In certain embodiments, more than one antibody-fluorophore conjugate will be brought into contact with the biological sample. An advantage of the direct conjugation of the fluorescence signal generator to the antibody is the high potential for correct identification of an antibody based on a fluorescence signal alone. Another perceived advantage of direct conjugation of antibodies to fluorophores is that the binding of antibody to the antigen simultaneously, or substantially simultaneously, achieves the fluorescent labeling step, potentially saving time.

Depending on the particular application incubation times to permit sufficient detection may vary. In certain embodiments, incubation times to permit sufficient detection may include overnight, 1 minute to 1 hour, 5 minutes to 20 minutes, 30 minutes to 1 hour, 20 minutes to 2 hours, 1 to 4 hours, 3 to 8 hours, or 6 to 12 hours.

It is common in flow cytometry of complex cellular samples to detect two or more surface antigens in a multiplex experiment, as a method to distinguish between cells with overlapping patterns of surface antigen (e.g., marker) expression.

Many fluorophores have broad emission spectra (the range of wavelengths over which they emit fluorescent light), so that in multiplexed studies employing antibodies labeled with, for example, two fluorophores (1 and 2) the flow cytometer detection channel dedicated to detection of fluorophore 1's emission may also “see” a relatively small amount of the light emitted by fluorophore 2. This is often referred to as spillover. Some flow cytometers provide so-called ‘compensation’ mechanisms to correct for this spillover either electronically or via software, but compensation often lacks sufficient accuracy and, in the case of a very bright signal from fluorophore 1 spilling over into a channel observing a comparatively dim signal from fluorophore 2 the unavoidable consequence of compensation is often an increase in the coefficient of variation of the fluorophore 2 signal, which presents as a broadening of the apparent intensity distribution of fluorophore 2's signal. This broadening of fluorophore 2's intensity distribution constitutes an artifact which it is desirable to avoid in many instances, as the artifact makes it difficult and sometime impossible, to determine the percentage of cells in the sample which express the antigen reported by the fluorophore 2-labeled antibody with sufficient accuracy. Certain embodiments of the present disclosure are directed to allowing flow cytometrists to optimize, or substantial optimize, improve or fine tune the brightness of a first labeled antibody fluorophore 1's fluorescence to minimize, substantially minimize or reduce its spillover into the detection channel for a second labeled antibody fluorophore 2. This may also be accomplished in assays that have 3, 4, 5, 6, 7, 8, 9, or more fluorophores that may be affected by the spillover of one or more other fluorophores.

### 4.5 Binding Moieties

In certain embodiments of a method as described herein, the oxidation-sensitive fluorophore-treated granulocytes are contacted with one or more binding moieties. In some embodiments, the one or more binding moieties are detectable by flow cytometry. In some embodiments, the binding moiety comprises a biomolecule, a synthetic molecule, a biopolymer, or a portion of the biomolecule, synthetic molecule, or biopolymer. Suitable binding moiety may include, but is not limited to, an antibody, antibody-fragment (e.g., an antigen-binding fragment, Fab, F(ab′)2, F(ab′), scFv, di-scFv, sdAb, minibody, diabody, triabody), genetically-modified antibody, genetically-modified antibody-fragment, antigen, a protein, a peptide, a carbohydrate, a nuclear receptor, a small molecule, a drug or drug-like molecule, or combinations or derivatives thereof. The binding moiety may be capable of recognizing and binding a target. The binding moiety may also comprise a specific binding affinity for a target. In some embodiments, the binding moiety is specific for a target that is a cell surface marker. In certain embodiments, the binding moiety specifically binds to or is specific for a target antigen present on one or more granulocyte subpopulations. In some embodiments, the binding moiety comprises an multispecific antibody. The binding moiety may comprise one or more oligonucleotides, for example, may be conjugated to one or more oligonucleotides. The binding moiety may comprise a spacer group. The binding moiety may also comprise detectable moiety (e.g., a fluorophore, fluorophore-reactive enzyme, or radiolabel).

In certain embodiments, the binding moiety is an antibody or antigen-binding fragment thereof, which is detectable by flow cytometry. In some embodiments, the antibody is selected from an antibody having at least two antigen or epitope binding sites, single polypeptide chain antibody, bispecific antibody (e.g. quadromes, triomes), interspecies hybrid antibody, or a molecule that has been chemically modified and may be regarded as a derivative of such molecule and which may be prepared either by methods of antibody production or by DNA recombination, using hybridoma techniques or antibody engineering or synthetically or semisynthetically.

A suitable antibody may be produced through a variety of methods. For example, various animals may be immunized to generate polyclonal antibodies by injecting them with an antigen, for example the target biological molecule, or another molecule sharing an epitope of the target biological molecule. In certain embodiments, the target biological molecule is a cell surface marker of a granulocyte. Such antigen molecules may be of natural origin or obtained by DNA recombination or synthetic methods, or fragments thereof and the desired antibodies obtained from the resulting sera may be purified. In some embodiments, the animal is a transgenic animal engineered for producing antibodies. In some embodiments, the antibody comprises a monoclonal antibody. In some embodiments, the antibody is human or humanized. In some embodiments, the antibody is not human or humanized.

In certain embodiments, the antibody is a modified antibody comprising an a histidine-rich region. The modified antibody may comprise an antibody that is exclusive of having a histidine-rich region. The modified antibody may comprise an antibody that is of the IgG type antibody or the IgM type antibody. The modified antibody may comprise one or more molecular tags, for example, but not limited to, a poly-histidine tag, a Flag Tag, a Myc tag, or a peptide tag that an antibody has been raised against. The modified antibody may comprise a poly-histidine fusion protein. The modified antibody may comprise one or more spacer groups, for example, such as a polyethylene glycol (PEG) or a polyethylene oxide group (PEO). The modified antibody may comprise one or moieties that include a reactive group.

Certain embodiments provide systems and/or methods that allow users to choose one or more optimal degrees of labeling for one or more binding moieties, thereby avoiding spillover errors in multiplexed immunoassays. For example, for use in multiplexed flow cytometry. Antibodies—biological proteins exhibiting high-affinity binding of single target molecules—are widely employed throughout biological research, clinical diagnostics, pharmaceutical drug discovery, and other disciplines to enable immunoassays to detect and quantify molecules of interest (e.g., antigens). Typically, an antibody employed in immunoassays must be labeled with another molecule to render them detectable; frequently, the labels employed are fluorescent molecules, which emit light over characteristic wavelength ranges (e.g., colors). Numerous fluorophores are commercially available today as antibody labels, covering the color gamut from deep red to violet. In ‘multiplexed’ assays, which aim to detect two or more different analytes in the same sample, two or more different antibodies are typically employed together, the two more different antibodies are labeled with different colored fluorophores to allow them to be detected individually. Multiplexed assays are increasingly common in flow cytometry, where multiply different analytes may be detected in the assay.

In certain embodiments of a method as described herein, the one or more binding moieties bind to a target selected from 2D7, Calprotectin (S100A8/A9), CD9, CD10, CD11a, CD11b, CD11c, CDw12, CD13, CD14, CD15, CD16, CD16b, CD16/32, CD17, CD18, CD22, CD23, CD24, CD25, CD29, CD31, CD32, CD32a, CD32b, CD32c, CD33, CD35, CD37, CD38, CD43, CD44, CD45, CD45RB, CD45RO, CD46, CD47, CD49d, CD50, CD53, CD55, CD58, CD59, CD60a, CD62L, CD63, CD64, CD64a, CD65, CD65s, CD66a, CD66b, CD66c, CD66d, CD68, CD69, CD75S, CD82, CD85A, CD85D, CD85K, CD87, CD88 (C5a receptor), CD89, CD92, CD93, CD95, CD97, CD98, CD100, CD101, CD107a, CD107b, CD112 (Nectin-2), CD114 (G-CSFR), CD116, CD119, CD120a, CD120b, CD123 (IL3Ra), CD125, CD126, CD130, CD131, CD132, CD139, CD141, CD147, CD148, CD153, CD154 (CD40 ligand), CD156a, CD156b, CD157, CD162, CD164, CD170 (SiglecF), CD171, CD172a, CD177, CD178, CD181 (CXCR1), CD182, CD183, CD192 (CCR2), CD193 (CCR3), CD195, CD203c, CD217, CD218, CD218a, CD218b, CD220, CD221, CD222, CD230, CD232, CD244, CD256, CD257, CD258, CD261, CD262, CD263, CD264, CD268, CD270, CD274, CD275, CD281 (TLR1), CD282 (TLR2), CD284 (TLR4), CD286 (TLR6), CD289 (TLR9), CD290, CD294, CD295, CD298, CD302, CD305, CD312, CD314, CD321, CD328, CD329, CD352, CD354, CD360, CD362, C/EBP alpha, CRTH-2, EMR1, FceR1, GATA-2, Ly-6G (Gr-1), or Siglec-8. In some embodiments, the one or more binding moieties bind to a target selected from CD11b, C16, C16b, CD33, CD63, CD123, CD193, or CD203c. In some embodiments of the method, each of the one or more binding moieties binds to a different target.

In certain embodiments, the oxidation-sensitive fluorophore-treated granulocytes are basophils. In certain embodiments, the oxidation-sensitive fluorophore-treated granulocytes are contacted with one or more binding moieties that each bind to a different target selected from 2D7, CD9, CD11a, CD11b, CD13, CD15, CD16, CD16/32, CD22, CD25, CD32, CD33, CD38, CD43, CD45, CD49b, CD63, CD69, CD88 (C5a receptor), CD123 (IL3Ra), CD125, CD154 (CD40 ligand), CD192 (CCR2), CD203c, CD218 (IL-18R), CD282 (TLR2), CD284 (TLR4), CD286 (TLR6), CD294 (CRTH2), CD281 (TLR1), CD289 (TLR9), C/EBP alpha, CRTH-2, FceR1, or GATA-2. In specific embodiments, the oxidation-sensitive fluorophore-treated granulocytes are contacted with one or more binding moieties that each bind to a different target selected from CD16, CD63, CD123, or CD203c.

In certain embodiments, the oxidation-sensitive fluorophore-treated granulocytes are eosinophils. In certain embodiments, the oxidation-sensitive fluorophore-treated granulocytes are contacted with one or more binding moieties that each bind to a different target selected from CD9, CD11b, CD13, CD15, CD16, CD16/32, CD24, CD32, CD33, CD35, CD43, CD45, CD49d, CD63, CD64, CD66b, CD116, CD123, CD125, CD126, CD170 (SiglecF), CD193 (CCR3), CD244, EMR1, FceR1, Siglec-8. In specific embodiments, the oxidation-sensitive fluorophore-treated granulocytes are contacted with one or more binding moieties that each bind to a different target selected from CD16, CD63, or CD193.

In certain embodiments, the oxidation-sensitive fluorophore-treated granulocytes are neutrophils. In certain embodiments, the oxidation-sensitive fluorophore-treated granulocytes are contacted with one or more binding moieties that each bind to a different target selected from Calprotectin (S100A8/A9), CD10, CD11b, CD13, CD15, CD16, CD16/32, CD17, CD18, CD24, CD32, CD33, CD35, CD43, CD44, CD49d, CD63, CD66a, CD66b, CD66c, CD66d, CD89, CD93, CD112 (Nectin-2), CD114 (G-CSFR), CD116, CD123, CD157, CD177, CD181 (CXCR1), CD193, CD281 (TLR1), CD282 (TLR2), CD284 (TLR4), CD286 (TLR6), CD289 (TLR9), or Ly-6G (Gr-1). In specific embodiments, the oxidation-sensitive fluorophore-treated granulocytes are contacted with one or more binding moieties that each bind to a different target selected from CD11b, CD16, CD33, CD63, CD123, or CD193.

In certain embodiments, the oxidation-sensitive fluorophore-treated granulocytes are contacted with an antibody that binds to CD63. In some embodiments, a granulocyte is determined to be activated when it is CD63(+). In specific embodiments, a basophil is determined to be activated when it is CD63(+). In specific embodiments, a eosinophil is determined to be activated when it is CD63(+). In specific embodiments, a neutrophil is determined to be activated when it is CD63(+).

In certain embodiments, the oxidation-sensitive fluorophore-treated granulocytes are contacted with an anti-human CD11b antibody.

In certain embodiments, the oxidation-sensitive fluorophore-treated granulocytes are contacted with an anti-human CD16 antibody. In some embodiments, the anti-human CD16 antibody is CD16 Monoclonal Antibody (eBioCB16 (CB16)) (Thermo Fisher Scientific Cat. No. 12-0168-42). In some embodiments, the anti-human CD16 antibody comprises the VH-CDR1, VH-CDR2, VH-CDR3, VL-CDR1, VL-CDR2, VL-CDR3 of the anti-CD16 antibody CD16 Monoclonal Antibody (eBioCB16 (CB16)) (Thermo Fisher Scientific Cat. No. 12-0168-42). In some embodiments, the anti-human CD16 antibody competes for binding with CD16 Monoclonal Antibody (eBioCB16 (CB16)) (Thermo Fisher Scientific Cat. No. 12-0168-42).

In certain embodiments, the oxidation-sensitive fluorophore-treated granulocytes are contacted with an anti-human CD33 antibody. In some embodiments, the anti-human CD33 antibody is PE-Cy7 Mouse Anti-Human CD33 (BD Cat. No. 333946). In some embodiments, the anti-human CD16 antibody comprises the VH-CDR1, VH-CDR2, VH-CDR3, VL-CDR1, VL-CDR2, VL-CDR3 of the anti-CD33 antibody PE-Cy7 Mouse Anti-Human CD33 (BD Cat. No. 333946). In some embodiments, the anti-human CD33 antibody competes for binding with PE-Cy7 Mouse Anti-Human CD33 (BD Cat. No. 333946).

In certain embodiments, the oxidation-sensitive fluorophore-treated granulocytes are contacted with an anti-human CD63 antibody. In some embodiments, the anti-human CD63 antibody is CD63 Monoclonal Antibody (H5C6), PE, eBioscience (Thermo Fisher Scientific Cat. No. 12-0639-42). In some embodiments, the anti-human CD63 antibody comprises the VH-CDR1, VH-CDR2, VH-CDR3, VL-CDR1, VL-CDR2, VL-CDR3 of the anti-CD63 antibody CD63 Monoclonal Antibody (H5C6), PE, eBioscience (Thermo Fisher Scientific Cat. No. 12-0639-42). In some embodiments, the anti-human CD63 antibody competes for binding with CD63 Monoclonal Antibody (H5C6), PE, eBioscience (Thermo Fisher Scientific Cat. No. 12-0639-42).

In certain embodiments, the oxidation-sensitive fluorophore-treated granulocytes are contacted with an anti-human CD123 antibody. In some embodiments, the anti-human CD123 antibody is CD123 Monoclonal Antibody (6H6), PE-Cyanine5, eBioscience (Thermo Fisher Scientific Cat. No. 15-1239-42). In some embodiments, the anti-human CD123 antibody comprises the VH-CDR1, VH-CDR2, VH-CDR3, VL-CDR1, VL-CDR2, VL-CDR3 of the anti-CD123 antibody CD123 Monoclonal Antibody (6H6), PE-Cyanine5, eBioscience (Thermo Fisher Scientific Cat. No. 15-1239-42). In some embodiments, the anti-human CD123 antibody competes for binding with CD123 Monoclonal Antibody (6H6), PE-Cyanine5, eBioscience (Thermo Fisher Scientific Cat. No. 15-1239-42).

In certain embodiments, the oxidation-sensitive fluorophore-treated granulocytes are contacted with an anti-human CD193 antibody. In some embodiments, the anti-human CD193 antibody is PerCP-Cy5.5 Mouse Anti-Human CD193 clone 5E8 (BD Cat. No. 564189). In some embodiments, the anti-human CD193 antibody comprises the VH-CDR1, VH-CDR2, VH-CDR3, VL-CDR1, VL-CDR2, VL-CDR3 of the anti-CD193 antibody PerCP-Cy5.5 Mouse Anti-Human CD193 clone 5E8 (BD Cat. No. 564189). In some embodiments, the anti-human CD193 antibody competes for binding with PerCP-Cy5.5 Mouse Anti-Human CD193 clone 5E8 (BD Cat. No. 564189).

In certain embodiments, the oxidation-sensitive fluorophore-treated granulocytes are contacted with an anti-human CD203c antibody.

In certain embodiments, the oxidation-sensitive fluorophore-treated granulocytes are contacted with an anti-histone H3 antibody. In some embodiments, the anti-histone H3 antibody is Histone H3 (Citrullinated R2+R8+R17) Monoclonal Antibody (Cayman Chemical Cat. No. 17939). In some embodiments, the anti-histone H3 antibody comprises the VH-CDR1, VH-CDR2, VH-CDR3, VL-CDR1, VL-CDR2, VL-CDR3 of the anti-Histone H3 (Citrullinated R2+R8+R17) Monoclonal Antibody (Cayman Chemical Cat. No. 17939). In some embodiments, the anti-histone H3 antibody competes for binding with Histone H3 (Citrullinated R2+R8+R17) Monoclonal Antibody (Cayman Chemical Cat. No. 17939).

In certain embodiments of a method as described herein, the oxidation-sensitive fluorophore-treated granulocytes are contacted with a primary, secondary, or tertiary antibody. The suitable primary antibody may contain an antigen binding region which can specifically bind to an antigen target (e.g., cell surface marker) in a sample, such as an immunohistochemistry sample, may be employed. For example, a primary antibody may be comprised within a primary binding moiety or a primary molecular probe. A suitable secondary antibody may contain an antigen binding region which can specifically bind to the primary antibody, for example, the constant region of the primary antibody. The secondary antibody may be conjugated to a polymer. The polymer may be conjugated with between about 2-20 secondary antibodies, or may be conjugated with between about 1-5 tertiary antibodies, such as 1, 2, 3, 4, or 5 tertiary antibodies. The secondary antibody may act as a secondary binding moiety, while in other embodiments, the secondary antibody may act as molecular probe, recognizing the target, such as an antigen, indirectly through a primary antibody. A suitable tertiary antibody may contain an antigen binding region which can specifically bind to the secondary antibody, for example, a constant region of the secondary antibody, or a hapten linked to the secondary antibody or a polymer conjugated to the secondary antibody. For example, the tertiary antibody may be conjugated to a polymer, such as between about 1-20 tertiary antibodies. The polymer may be conjugated with between about 1-5 tertiary antibodies, such as 1, 2, 3, 4, or 5 tertiary antibodies. The tertiary antibody may act as a tertiary binding moiety. In other embodiments, the tertiary antibody may act as molecular probe, recognizing the target, such as an antigen, indirectly through a primary antibody and a secondary antibody.

In certain embodiments, the secondary antibody specifically binds a primary antibody, wherein the primary antibody is specific for 2D7, Calprotectin (S100A8/A9), CD9, CD10, CD11a, CD11b, CD11c, CDw12, CD13, CD14, CD15, CD16, CD16b, CD16/32, CD17, CD18, CD22, CD23, CD24, CD25, CD29, CD31, CD32, CD32a, CD32b, CD32c, CD33, CD35, CD37, CD38, CD43, CD44, CD45, CD45RB, CD45RO, CD46, CD47, CD49d, CD50, CD53, CD55, CD58, CD59, CD60a, CD62L, CD63, CD64, CD64a, CD65, CD65s, CD66a, CD66b, CD66c, CD66d, CD68, CD69, CD75S, CD82, CD85A, CD85D, CD85K, CD87, CD88 (C5a receptor), CD89, CD92, CD93, CD95, CD97, CD98, CD100, CD101, CD107a, CD107b, CD112 (Nectin-2), CD114 (G-CSFR), CD116, CD119, CD120a, CD120b, CD123 (IL3Ra), CD125, CD126, CD130, CD131, CD132, CD139, CD141, CD147, CD148, CD153, CD154 (CD40 ligand), CD156a, CD156b, CD157, CD162, CD164, CD170 (SiglecF), CD171, CD172a, CD177, CD178, CD181 (CXCR1), CD182, CD183, CD192 (CCR2), CD193 (CCR3), CD195, CD203c, CD217, CD218, CD218a, CD218b, CD220, CD221, CD222, CD230, CD232, CD244, CD256, CD257, CD258, CD261, CD262, CD263, CD264, CD268, CD270, CD274, CD275, CD281 (TLR1), CD282 (TLR2), CD284 (TLR4), CD286 (TLR6), CD289 (TLR9), CD290, CD294, CD295, CD298, CD302, CD305, CD312, CD314, CD321, CD328, CD329, CD352, CD354, CD360, CD362, C/EBP alpha, CRTH-2, EMR1, FceR1, GATA-2, Ly-6G (Gr-1), or Siglec-8.

In certain embodiments, the tertiary antibody is specific for a secondary antibody. In some embodiments, the secondary or tertiary antibody is Goat Anti-Mouse IgG:SureLight APC (Cayman Chemical Cat. No. 16587). In some embodiments, the secondary or tertiary antibody comprises the VH-CDR1, VH-CDR2, VH-CDR3, VL-CDR1, VL-CDR2, VL-CDR3 of the antibody Goat Anti-Mouse IgG:SureLight APC (Cayman Chemical Cat. No. 16587). In some embodiments, the secondary or tertiary antibody competes for binding with antibody Goat Anti-Mouse IgG:SureLight APC (Cayman Chemical Cat. No. 16587).

In specific embodiments, when more than one antibody detectable by flow cytometry is used in the method, the label for each antibody is different. In specific embodiments, the antibody detectable by flow cytometry comprises a binding site for a secondary antibody, wherein the secondary antibody is detectable by flow cytometry or is a binding site for a tertiary antibody that is detectable by flow cytometry.

### 4.6 Stimulants

In certain embodiments of a method as described herein, the exogenous stimulant is selected from an environmental stimulant, foodstuff stimulant, or medicament stimulant.

In certain embodiments, the granulocytes are contacted with or exposed to from about 1 ng/mL to about 100 μg/mL of exogenous stimulant. In specific embodiments, the granulocytes are contacted with or exposed to from about 0.3 μg/mL to about 3 μg/mL of exogenous stimulant.

### 4.6.1 Environmental Stimulants

In certain embodiments, the environmental stimulant is an environmental allergen. In some embodiments, the environmental stimulant selected from pollen, plant, insect, dust mite, cockroach, animal, venom, mold, latex, metal, vitamin, or mineral.

In certain embodiments, the pollen is selected from a tree or shrub pollen, flower pollen, grass pollen, or weed pollen. In specific embodiments, the tree or shrub pollen is selected from azalea pollen, alder pollen, ash pollen, aspen pollen, beech pollen, birch pollen, boxelder pollen, boxwood pollen, cedar pollen, cottonwood pollen, cypress pollen, elm pollen, hibiscus pollen, hydrangea pollen, hickory pollen, jasmine vine, juniper pollen, maple pollen, maple sycamore pollen, mountain juniper, mulberry pollen, oak pollen, olive pollen, pecan pollen, poplar pollen, viburnum pollen, walnut pollen, willow pollen, or wisteria pollen. In specific embodiments, the flower pollen is selected from begonia pollen, iris pollen, cactus pollen, clematis pollen, crocus pollen, chenille pollen, daffodil pollen, dusty miller pollen, geranium pollen, hosta pollen, impatiens pollen, lily pollen, verbena pollen, pansy pollen, columbine pollen, petunia pollen, periwinkle pollen, rose pollen, phlox pollen, snapdragon pollen, salvia pollen, thrift pollen, tulip pollen, or zinnia pollen. In specific embodiments, the grass pollen is selected from Bahia pollen, Bermuda pollen, fescue pollen, Johnson pollen, Kentucky blue pollen, orchard pollen, redtop pollen, ryegrass pollen, salt grass pollen, sweet vernal pollen, or Timothy pollen. In specific embodiments, the weed pollen is selected from ragweed pollen, burning bush pollen, cocklebur pollen, English plantain pollen, goosefoot pollen, mugwort pollen, Lamb's-quarters pollen, mugwort pollen, nettle pollen, pigweed pollen, Russian thistle pollen, sagebrush pollen, saltwort pollen, tumbleweed pollen, or wall pellitory pollen.

In certain embodiments, the insect stimulant is an insect allergen. In certain embodiments, the insect stimulant is an intact insect, or a fragment thereof. In certain embodiments, the insect stimulant is feces, saliva, or urine. In some embodiments, the insect stimulant is from a dust mite. In some embodiments, the insect stimulant is from a cockroach. In some embodiments, the insect stimulant is from an ant, bee, caterpillar, centipede, hornet, mosquito, scorpion, snail, spider, wasp, or yellow jacket. In certain embodiments, the dust mite stimulant is an intact dust mite, or a fragment thereof. In certain embodiments, the dust mite stimulant is feces, saliva, or urine. In specific embodiments, dust mite stimulant is from a Dermatophagoides selected from Dermatophagoides farina, Dermatophagoides pteronyssinus, Dermatophagoides evansi, Dermatophagoides microceras, Dermatophagoides halterophilus, Dermatophagoides siboney, Dermatophagoides neotropicalis, Dermatophagoides alexfaini, Dermatophagoides anisopoda, Dermatophagoides chirovi, Dermatophagoides deanei, Dermatophagoides rwandae, Dermatophagoides scheremeteroskyi, Dermatophagoides scheremetewskyi, Dermatophagoides simplex, Euroglyphus maynei (Mayne's house dust mite), Euroglyphus longior, Hirstia domicola, Malayoglyphus carmelitus, Malayoglyphus intermedius, Pyroglyphus africanus, Sturnophagoides brasiliensis, or Blomia tropicalis. In certain embodiments, the cockroach stimulant is an intact cockroach, or a fragment thereof. In certain embodiments, the cockroach stimulant is feces, saliva, or urine. In specific embodiments, the cockroach stimulant is from a cockroach selected from American cockroach (Periplaneta americana), Brown-banded cockroach (Supella longipalpa), German cockroach (Blattella germanica), Oriental cockroach (Blatta orientalis), or Smokybrown cockroach (Periplaneta fuliginosa).

In certain embodiments, the animal stimulant is an animal allergen. In certain embodiments, the animal stimulant is animal dander, feces, hair, saliva, or urine. In certain embodiments, the animal stimulant is selected from a pet animal and/or livestock animal. In specific embodiments, the animal stimulant is from an animal selected from a cat, dog, rodent, mouse, rat, or rabbit. In specific embodiments, the animal stimulant is cat dander or dog dander.

In certain embodiments, the venom stimulant is a venom allergen. In certain embodiments, the venom stimulant is an animal venom. In certain embodiments, the animal venom is from a fish, amphibian, reptile, or mammal. In specific embodiments, the animal venom is from a fish selected from a stingray, shark, chimaera, catfish, spiny-rayed fish (Acanthomorpha), scorpionfish, stonefish, gurnard perch, blennies, rabbitfish, surgeonfish, velvetfish, toadfish, coral croucher, red velvetfish, scat, rockfish, deepwater scorpionfish, waspfish, weever, or stargazer. In specific embodiments, the animal venom is from an amphibian selected from a salamander or frog. In specific embodiments, the animal venom is from a reptile selected from a snake, lizard (e.g., Mexican beaded lizard or monitor lizard), iguana, Gila monster, or Komodo dragon. In specific embodiments, the animal venom is from a mammal selected from a solenodon, shrew, bat, platypus, or slow lorise. In certain embodiments, the venom allergen is an insect venom. In specific embodiments, the insect venom is from an insect selected from an ant, bee, caterpillar, centipede, hornet, mosquito, scorpion, snail, spider, wasp, or yellow jacket.

In certain embodiments, the mold stimulant is a mold allergen. In certain embodiments, the mold stimulant is from a mold selected from Alternaria, Aspergillus, Candida, Cladosporium, Mucor, Penicillium, or Stachybotrys. In specific embodiments, the mold stimulant is from a mold selected from Alternaria alternata, Aspergillus fumigatus, Candida albicans, Cladosporium herbarum, Mucor racemosus, or Penicillium chrysogenum.

In certain embodiments, the latex stimulant is a latex allergen. In certain embodiments, the latex stimulant is from natural latex or synthetic latex.

In certain embodiments, the metal stimulant is a metal allergen. In certain embodiments, the metal stimulant is from a metal selected from nickel, cobalt, copper, chromium, titanium, or zinc.

In certain embodiments, the vitamin stimulant is a vitamin allergen. In certain embodiments, the vitamin stimulant is from a vitamin B or vitamin D. In specific embodiments, the vitamin stimulant is from vitamin B-12. In specified embodiments, the vitamin stimulant is from vitamin D-3. In specific embodiments, the vitamin stimulant is bismuth oxychloride.

### 4.6.2 Foodstuff Stimulants

In certain embodiments, the foodstuff stimulant is a foodstuff allergen. In some embodiments, the foodstuff stimulant is selected from a seed, nut, egg, dairy product, oil, condiment, fruit, vegetable, cereal, grain, legume, meat, wheat, soy, seafood, herb, or spice. In certain embodiments, the seed stimulant is selected from buckwheat seed, mustard seed, poppy seed, pumpkin seed, sesame seed, or sunflower seed. In certain embodiments, the nut stimulant is a tree nut stimulant. In certain embodiments, the nut stimulant is selected from almond nut, Brazil nut, cashew nut, chestnut, hazelnut, macadamia nut, pecan nut, pine nut, pistachio, or walnut. In certain embodiments, the egg stimulant is a hen's egg. In certain embodiments, the dairy product stimulant is selected from a cow milk product, a goat milk product, or a sheep milk product. In certain embodiments, the fruit stimulant is selected from acerola, apple, apricot, avocado, banana, cherry, Chinese gooseberry, coconut, date, fig, garden plum, grape, kiwi, lychee, mango, melon, orange, passion fruit, peach, pear, persimmon, pineapple, pomegranate, prune, strawberry, or tomato. In certain embodiments, the vegetable stimulant is selected from asparagus, avocado, bell pepper, cabbage, carrot, celery, lettuce, potato, pumpkin, turnip, or zucchini. In certain embodiments, the cereal or grain stimulant is selected from barley, corn, gluten, oat, rice, rye, or wheat. In certain embodiments, the legume allergen is selected from chickpea, lentil, lupin, peanut, pea, soy, or soybean. In certain embodiments, the seafood stimulant is a fish allergen or shellfish allergen. In specific embodiments, the fish stimulant is selected from Alaska pollock, carp, cod, dogfish, mackerel, salmon, sole, or tuna. In specific embodiments, the shellfish stimulant is selected from abalone, crab, horned turban, limpet, lobster, marine snail, mussel (blue or tropical green), octopus, oyster, scallop, shrimp, snail, squid, or whelk. In certain embodiments, the spice or herb stimulant is selected from anis, artichoke, celery, coriander, cumin, dandelion, Echinacea, fennel, hibiscus, parsley, or ragweed.

### 4.6.3 Medicament Stimulants

In certain embodiments, the medicament stimulant is medicament allergen. In certain embodiments, the medicament stimulant is selected from a drug compound, vaccine, adjuvant, or pharmaceutical excipient. As a skilled artisan would recognize, certain medicament stimulants may be classified in one or more of these categories (e.g., both as an excipient and an adjuvant).

### 4.6.3.1 Drug Stimulants

In certain embodiments, the medicament stimulant is a drug compound selected from an antibiotic (e.g., penicillin), sulfonamide, cephasporin, anesthetic (e.g., Novocain or lidocaine), anticonvulsant, acetylsalicylic acid, aspirin, ibuprofen, NSAID, morphine, chemotherapy drug, or radio contrast media (RCM).

### 4.6.3.2 Vaccine Stimulants

In certain embodiments, the medicament stimulant is a vaccine. In specific embodiments, the vaccine is selected from a COVID-19 vaccine (e.g., Comirnaty®/BNT162b2; SPIKEVAX®/mRNA-1273; Ad26.COV2.S/JNJ-78436735); Adenovirus Type 4 and Type 7 vaccine; Anthrax vaccine (e.g., Biothrax®); BCG vaccine (e.g., BCG Vaccine®, TICE BCG®); Cholera vaccine (e.g., Vachora®); Dengue Tetravelent vaccine (e.g., DENGVAXIA®); Diphtheria & Tetanus Toxoid vaccine (e.g., TDVAX®; TENIVAC®); Diphtheria & Tetanus Toxoids and Acellular Pertussis vaccine (e.g., Infanrix®; DAPTACEL®; Adacel®; Boostrix®); Diphtheria & Tetanus Toxoids & Acellular Pertussis and Poliovirus vaccine (e.g., KINRIX®; Quadracel®); Diphtheria & Tetanus Toxoids and Acellular Pertussis and Hepatitis B and Poliovirus vaccine (e.g., Pediarix®); Diphtheria & Tetanus Toxoids and Acellular Pertussis and Poliovirus and Haemophilus b Conjugate vaccine (e.g., Pentacel®); Diphtheria & Tetanus Toxoids and Acellular Pertussis and Poliovirus and Haemophilus b Conjugate and Hepatitis B vaccine (e.g., VAXELIS®); Ebola Zaire vaccine (e.g., ERVEBO®); Haemophilus b Conjugate vaccine (e.g., PedvaxHIB®; ActHIB®, Hiberix®); Hepatitis A vaccine (e.g., Havrix®; VAQTA®); Hepatitis B vaccine (e.g., Recombivax HB®; PREHEVBRIO®; Engerix-B®; HEPLISAV-B®); Hepatitis A and Hepatitis B vaccine (Twinrix®); Human Papillomavirus Quadrivalent (Types 6, 11, 16, 18) vaccine (e.g., Gardisil®); Human Papillomavirus 9-Valent vaccine (e.g., Gardisil 9®); Human Papillomavirus Bivalent (Types 16, 18) vaccine (e.g., Cervaris®); Influenza A (H1N1) vaccine; Influenza A (H5N1) vaccine (e.g., AUDENZ®); Influenza vaccine (e.g., Fluad®; Fluad Quadrivalent®, Afluria Quadrivalent®, Quadrivalent Southern Hemisphere®; Flucelvax Quadrivalent®; Flulaval Quadrivalent®); Influenza Virus vaccine (Trivalent, Types A and B) (e.g., Afluria®, Afluria Southern Hemisphere®; FluLaval®; FluMist®; Fluarix®; Fluvirin®; Agriflu®; Fluzone®; Fluzone High-Dose®; Fluzone Intradermal®; Flucelvax®); Influenza (Trivalent) vaccine (e.g., Flubok®); Influenza (Quadrivalent) vaccine (e.g., Flublok Quadrivalent®); Influenza Virus Vaccine (Quadrivalent, Types A and Types B) (e.g., FluMist Quadrivalent®; Fluarix Quadrivalent®; Fluzone Quadrivalent®); Japanese Encephalitis Virus vaccine (e.g., Ixiaro®); Measles, Mumps, and Rubella Virus vaccine (e.g., M-M-R II®); Measles, Mumps, Rubella, and Varicella Virus vaccine (e.g., Proquad®); Meningococcal (Groups A, C, Y, and W-135 combined) Oligosaccharide Diphtheria CRM197 Conjugate vaccine (e.g., Menveo®); Meningococcal (Groups A, C, Y and W-135 combined) Polysaccharide Diphtheria Toxoid Conjugate vaccine (e.g., Menactra®); Meningococcal Group B vaccine (e.g., BEXSERO®; TRUMENBA®); Meningococcal Polysaccharide (Groups A, C, Y and W-135 Combined) vaccine (e.g., MenQuadfi®); Plague vaccine; Pneumococcal vaccine (e.g., Pneumovax 23®); Pneumococcal 13-valent Conjugate vaccine (e.g., Prevnar 13@); Pneumococcal 15-valent Conjugate vaccine (e.g., VAXNEUVANCE®); Pneumococcal 20-valent Conjugate vaccine (e.g., Prevnar 20@); Poliovirus vaccine (e.g., Poliovax®; IPOL®); Rabies vaccine (e.g., Imovax®; RabAvert®); Rotavirus vaccine (e.g., ROTARIX®; RotaTeq®); Smallpox vaccine (e.g., ACAM2000@); Smallpox & Monkeypox vaccine (e.g., JYNNEOS®); Tick-Borne Encephalitis vaccine (e.g., TICOVAC®); Typhoid vaccine (e.g., Vivotif®; Typhim Vi®); Varicella Virus vaccine (e.g., Varivax®); Yellow Fever Vaccine (e.g., YF-Vax®); Zoster vaccine (e.g., Zostavax®; SHINGRIX®).

In certain embodiments, the medicament stimulant is a vaccine component. In certain embodiments, the medicament stimulant is a component of the BNT162b2 (Pfizer) COVID-19 vaccine. In specific embodiments, the medicament stimulant is selected from (4-hydroxybutyl)azanediyl)bis(hexane-6,1-diyl)bis(2-hexyldecanoate)] (ALC-0315), 2-[(polyethylene glycol)-2000]-N,N-ditetradecylacetamide (ALC-0159), 1,2-Diastearoyl-sn-glycero-3-phosphocholine cholesterol (DSPC), potassium dihydrogen phosphate, sodium chloride, disodium hydrogen phosphate dihydrate, or sucrose. In specific embodiments, the medicament stimulant is selected from ALC0135, PEG-2000-DMG, ALC0159, or DSPC. In certain embodiments, the medicament stimulant is a component of the mRNA-1273 (Moderna) COVID-19 vaccine. In specific embodiments, the medicament stimulant is selected from a lipid (SM-102, 2,2-dimyristoyl-rac-glycero3-methoxypolyethylene glycol-2000 [PEG2000-DMG], cholesterol, and 1,2-diastearoyl-sn-glycero-3-phosphocholine [DSPC]), tromethamine, tromethamine hydrochloride, acetic acid, sodium acetate, or sucrose. In specific embodiments, the medicament stimulant is selected from DSPC, SM102, or PEG2000-DMG. In certain embodiments, the medicament stimulant is a component of the Ad26.COV2.S (Johnson & Johnson) COVID-19 vaccine. In specific embodiments, the medicament stimulant is selected from 2-hydroxypropyl-β-cyclodextrin (HBCD), citric acid monohydrate, ethanol, hydrochloric acid, polysorbate-80 (PS-80), sodium chloride, sodium hydroxide, or trisodium citrate dihydrate. In specific embodiments, the medicament stimulant is selected from ethanol, HBCD, or PS80.

### 4.6.3.3 Adjuvant Stimulants

In certain embodiments, the medicament stimulant is an adjuvant. In specific embodiments, the medicament stimulant is an adjuvant selected from an aluminum adjuvant (e.g., amorphous aluminum hydroxyphosphate sulfate, aluminum hydroxide, aluminum phosphate, potassium aluminum sulfate), AS04 (e.g., Monophosphoryl lipid A (MPL)+aluminum salt), MF59 (e.g., oil in water emulsion of squalene), ASO1 (e.g., monophosphoryl lipid A (MPL) and QS-21 in liposomal formulation), or CpG 1018 (cytosine phosphoguanine (CpG)). In some embodiments, the adjuvant is selected from Freund's adjuvant, a mineral gel (e.g., aluminum hydroxide), a surfactant (e.g., polyanion), a peptide, an oil emulsion, haemocyanin, dinitrophenol, or lysolecithin.

### 4.6.3.4 Pharmaceutical Excipient Stimulants

In certain embodiments, the medicament stimulant is a pharmaceutical excipient. In specific embodiments, the medicament stimulant is a pharmaceutical excipient selected from lactose, corn starch, PEG, povidone, carboxymethylcellulose, gelatin, brilliant blue, sunset yellow FCF, allura red, propylene glycol, peanut oil, gluten, or chemical dye (e.g., tartazine).

In certain embodiments, the medicament stimulant is selected from (4-hydroxybutyl)azanediyl)bis(hexane-6,1-diyl)bis(2-hexyldecanoate)] (ALC-0315); 1,2-Diastearoyl-sn-glycero-3-phosphocholine cholesterol (DSPC); 2-[(polyethylene glycol)-2000]-N,N-ditetradecylacetamide (ALC-0159); 2-hydroxypropyl-β-cyclodextrin (HBCD); acetic acid; citric acid monohydrate; disodium hydrogen phosphate dihydrate; ethanol hydrochloric acid; lipids (SM-102, 2,2-dimyristoyl-rac-glycero3-methoxypolyethylene glycol-2000 [PEG2000-DMG]; cholesterol; polysorbate-80; potassium dihydrogen phosphate; sodium acetate; sodium chloride; sodium hydroxide; sucrose; trisodium citrate dihydrate; tromethamine; or tromethamine hydrochloride.

### 4.7 Biological Samples

In certain embodiments of a method as described herein, the biological sample of the subject comprises a biological fluid. In certain embodiments, the biological fluid comprises an intravascular biological fluid, interstitial biological fluid, or intracellular biological fluid. In certain embodiments, the biological sample is selected from a blood sample, amniotic fluid sample, aqueous humor sample bone marrow sample, bronchoalveolar lavage sample, buccal swab sample, cerebrospinal fluid sample, earwax sample, fecal sample, gastric fluid sample, gastrointestinal fluid sample, liposuction sample, milk sample, nasal wash sample, peritoneal fluid sample, plasma sample, saliva sample, sebum sample, semen sample, serum sample, sputum sample, synovial fluid sample, tears sample, urine sample, vaginal fluid sample, or vitreous humor sample. In specific embodiments, the biological sample is a blood sample, optionally a peripheral blood or whole blood sample.

In certain embodiments, the biological sample is about 1 mL or less, about 500 μL or less, about 400 μL or less, about 200 μL or less, about 150 μL or less, about 125 μL or less, about 100 μL or less, about 75 μL or less, about 50 μL or less, about 25 μL or less, about 10 μL or less, about 5 μL or less, or about 1 μL or less. In certain embodiments, the biological sample is diluted prior to analysis. In certain embodiments, the biological sample is not diluted prior to analysis.

### 4.8 Immune Reactions

In certain embodiments of a method as described herein, an immune reaction of the subject to an exogenous stimulant is detected or predicted. The immune reaction may be characterized by immune reaction type or immune reaction severity. In some embodiments, the immune reaction type is non-allergic. In some embodiments, the immune reaction type is allergic. In some embodiments, the immune reaction type is anaphylaxis. In some embodiments, the immune reaction severity is mild. In some embodiments, the immune reaction severity is severe.

In certain embodiments, the immune reaction type is an immunoglobulin- or immune complex-mediated hypersensitivity. In some embodiments, the immune reaction type is an IgE-mediated hypersensitivity. In some embodiments, the immune reaction type is an IgG-mediated hypersensitivity. In some embodiments, the immune reaction type is an IgM-mediated hypersensitivity. In some embodiments, the immune reaction type is an immune complex-mediated hypersensitivity. In some embodiments, the immune reaction type is a cell-mediated hypersensitivity. In some embodiments, the immune reaction type is a Type I hypersensitivity. In some embodiments, the immune reaction type is a Type II hypersensitivity. In some embodiments, the immune reaction type is a Type III hypersensitivity. In some embodiments, the immune reaction type is a Type IV hypersensitivity.

In certain embodiments, the immune reaction occurs or is predicted to occur immediately, within 5 minutes to 2 hours, within 2 hours to 72 hours, or within 3 days and 7 days after exposure to an exogenous stimulant.

In certain embodiments, the immune reaction comprises one or more inflammatory symptoms. In some embodiments, the immune reaction comprises rash, fever, chills, irritability, muscle and joint pain, stomach upset, headache, fatigue, pain, redness, swelling, hives, flushing. In certain embodiments, the immune reaction comprises one or more of skin symptoms (e.g., itching, hives, flushing or facial swelling), breathing problems (e.g., shortness of breath, wheezing, cough), symptoms due to low blood pressure (e.g., confusion, disorientation, dizziness, lightheadedness, weakness, or fast heart rate), or gastrointestinal symptoms (e.g., nausea, vomiting, stomach cramps, or diarrhea).

### 4.9 Therapeutic Methods and Uses

In another aspect, provided herein are methods of treating a subject with a modified therapy. In some embodiments, a modified therapy comprises standard therapy for a particular disease or disorder that lacks one or more exogenous stimulants determined or predicted to cause an immune response in the subject. In some embodiments, the one or more exogenous stimulants determined or predicted to cause an immune response are substituted with a replacement component that is not determined or predicted to cause an immune response in the subject.

Such methods and uses as described herein include therapeutic methods and uses, for example, involving administration of therapeutic molecules or compositions containing the same, to a subject having a disease or disorder. In some embodiments, the composition is administered in an effective amount to effect treatment of the disease or disorder. Uses include uses of the compositions in such methods and treatments, and in the preparation of a medicament in order to carry out such therapeutic methods. In some embodiments, the methods are carried out by administering the compositions to the subject having or suspected of having the disease or condition. In some embodiments, the methods thereby treat the disease or disorder in the subject.

In some embodiments, the treatment provided herein cause complete or partial amelioration or reduction of a disease or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms include, but do not imply, complete curing of a disease or complete elimination of any symptom or effect(s) on all symptoms or outcomes.

As used herein, in some embodiments, the treatment provided herein delay development of a disease or disorder, e.g., defer, hinder, slow, retard, stabilize, suppress and/or postpone development of the disease (e.g., cancer or viral infection). This delay can be of varying lengths of time, depending on the history of the disease and/or individual being treated. As is evident to one skilled in the art, a sufficient or significant delay can, in effect, encompass prevention, in that the individual does not develop the disease or disorder. For example, a late stage cancer, such as development of metastasis, may be delayed. In another example, a viral infection may be delayed. In other embodiments, the method or the use provided herein prevents a disease or disorder.

### 4.10 Diagnostic and Detection Methods and Uses

In another aspect, provided herein are methods involving the use oxidation-sensitive fluorophores together with flow cytometry and/or microplate analysis for detection of immune response, prediction of immune reaction type and/or severity, or compatibility of a subject to/with an exogenous stimulant by ex vivo testing of a biological sample from the subject.

In some embodiments, a biological sample is contacted with an oxidation-sensitive fluorophore provided herein and oxidation of the fluorophore is determined or detected. When oxidation in the test sample is determined or detected as compared to a reference control of the same type, it indicates a positive reaction and may indicated the presence of an associated disease, disorder, immune reaction, or allergy. In some embodiments, the biological sample is from a human and may be from a diseased and/or healthy subject.

In certain embodiments, labeled antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. In other embodiments, antibodies are not labeled, and the presence thereof can be detected using a labeled antibody which binds to any of the antibodies.

### 4.11 Kits and Articles of Manufacture

Further provided are kits and articles of manufacture comprising any of the molecules, reagents, or compositions described herein. In some embodiments, a kit is provided which contains any one of the diagnostic reagents described herein and preferably provides instructions for its use.

The kits of the present application are in suitable packaging. Suitable packaging includes, but is not limited to, vials, bottles, jars, flexible packaging (e.g., sealed Mylar or plastic bags), boxes, and the like. Kits may optionally provide additional components such as buffers and interpretative information. The present application thus also provides articles of manufacture, which include vials (such as sealed vials), bottles, jars, flexible packaging, and the like.

The article of manufacture can comprise a container and a label or package insert on or associated with the container. Suitable containers include, for example, bottles, vials, syringes, boxes, etc. The containers may be formed from a variety of materials such as glass, plastic, cardboard, or paper. Generally, the container holds a composition which is effective as a reagent in a method described herein, and may have a sterile access port (for example the container may have a stopper pierceable by a hypodermic injection needle). The label or package insert indicates that the composition is used for determining the response of a subject to exogenous stimulant. The label or package insert will further comprise instructions for contacting the composition with a biological sample of the subject. The label may indicate directions for reconstitution (e.g., of a lyophilized reagent) and/or use. The container holding the pharmaceutical composition may be a multi-use vial, which allows for repeat administrations of the reconstituted formulation. Package insert refers to instructions customarily included in commercial packages of diagnostic products that contain information about the indications, usage, suitable concentrations and/or warnings concerning the use of such diagnostic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple units of the diagnostic reagents and instructions for use, packaged in quantities sufficient for storage and use in clinical laboratories, for example, a blood testing facility.

The disclosure is generally disclosed herein using affirmative language to describe the numerous embodiments. The disclosure also specifically includes embodiments in which particular subject matter is excluded, in full or in part, such as substances or materials, method steps and conditions, protocols, procedures, assays or analysis. Thus, even though the disclosure is generally not expressed herein in terms of what the disclosure does not include, aspects that are not expressly included in the disclosure are nevertheless disclosed herein.

In some aspects, provided herein is a kit for use in detecting the response of a subject an exogenous stimulant. In some embodiments, the kit is for testing a biological sample from a subject. In some embodiments, the kit comprises instructions for the use of one or more binding moieties to determine the subpopulation of granulocytes present in a biological sample. In some embodiments, the kit further comprises an oxidation-sensitive fluorophore. In some embodiments, the kit provides instructions for contacting the oxidation-sensitive fluorophore with a biological sample form a subject. In some embodiments, the kit provides instructions for determining response of granulocytes to an exogenous stimulant based on reactivity of the biological sample with an oxidation-sensitive fluorophore.

In some embodiments, the kit comprises one or more antibody or binding fragment selected from: an antibody or binding fragment that binds to a granulocyte degranulation marker; an antibody or binding fragment that binds to an activated neutrophil; or an antibody or binding fragment that binds to an activated basophil or eosinophil. In certain embodiments, the antibody is selected from an anti-CD63 antibody, an anti CD11b antibody, an anti CD193 antibody, or an anti CD203c antibody.

In certain aspects, provided is a reagent composition for flow cytometry determination of allergic response in a subject to an exogenous stimulant, comprising
(i) an oxidation-sensitive fluorophore; and
(ii) a collection of one or more binding moieties selected from:
   a) an anti-neutrophil binding moiety that binds to a neutrophil cell surface marker;
   b) an anti-eosinophil binding moiety that binds to a eosinophil cell surface marker; or
   c) an anti-basophil binding moiety that binds to a basophil cell surface marker,
wherein the oxidation-sensitive fluorophore is detectable by fluorometric measurement, and each of the one or more binding moieties are detectable by flow cytometry.

In certain aspects, provided is a kit comprising:
(i) an oxidation-sensitive fluorophore;
(ii) a collection of one or more binding moieties selected from:
   a) an anti-neutrophil antibody that binds to a neutrophil cell surface marker;
   b) an anti-eosinophil antibody that binds to a eosinophil cell surface marker; or
   c) an anti-basophil antibody that binds to a basophil cell surface marker; and
(iii) optionally, propidium iodide (PI);
(iv) optionally, an anti-citrullinated histone H3 (H3cit) antibody; and
(v) instructions for determining allergic response in a subject to an exogenous stimulant via flow cytometry using the DHR 123, collection of antibodies, and optionally PI of (i) to (ii) or (i) to (iii).

A number of embodiments of the disclosure have been described. Nevertheless, it will be understood that various modifications may be made without departing from the spirit and scope of the disclosure. Accordingly, the following examples are intended to illustrate but not limit the scope of the invention.

### 5. EXAMPLES

### 5.1 Example 1: Flow Cytometry Method to Detect the Activation of Granulocytes in Blood

This example describes the development of a flow cytometry approach for detecting the activation of granulocytes in blood following exposure to an exogenous stimulant, using oxidation-sensitive fluorophore staining (e.g., dihydrorhodamine 123).

### 5.1.1 Materials and Methods

See FIG. 1 and FIG. 2 for exemplary flow diagrams of the method. Whole peripheral blood samples were obtained from subjects and collected in lithium heparin tubes. Samples were diluted 1:100 in electrolyte solution supplemented with 10% heat-inactivated fetal-bovine serum (FBS). Samples were incubated with 5 μg of dihydrorhodamine 123 (DHR 123) at 37° C. under 5% CO₂ for 15 min. Cells were treated with 1 μM Phorbol-12-Myristate-13-Acetate (PMA), an exogenous stimulant, or an equal volume of PBS (negative control), and incubated for 30 min to 1 hr.

RBCs of the sample were lysed, fixed, and removed prior to flow cytometry analysis. Briefly, cells were lysed and partially fixed using 3% (v/v) lysis buffer and 1% (v/v) formaldehyde in phosphate-buffered saline (PBS). Samples were incubated for 2 min or until turbidity of the solution was resolved indicating essentially complete lysis of RBCs, and the sample centrifuged at 300×g for 5 min at room temperature (RT). RBC-containing supernatant was discarded, and remaining leukocytes were fixed using 3.7% (v/v) formaldehyde and centrifuged at 300×g at RT for 5 min. Samples were washed twice in FBS and centrifuged at 300×g at RT for 5 min. After washing, the cells were blocked with FC block (Invitrogen, USA) and 2% FBS for 30 min on ice and then incubated with staining buffer containing manufacturer-specified dilutions of fluorophore-conjugated antibodies specific for granulocytes (e.g., neutrophils, basophils, and/or eosinophils) or granulocyte activation (e.g., degranulation). Cells were washed twice and resuspended in PBS prior to analysis using a BD Accuri C6 Flow Cytometer (BD Biosciences, USA). Statistical analysis was performed using the FlowJo v10 (FlowJo, Oregon, USA).

Dead cells were gated and removed using PI(+) viability marker. The cell surface marker CD33 was used to distinguish the lymphocyte population from the monocyte and granulocyte populations. The cell surface marker CD16 was used to distinguish neutrophil and eosinophil populations from the monocytes and lymphocyte populations. The cell surface marker CD123 was used to distinguish the basophil population. Activated granulocytes were observed using the granulocyte activation surface marker CD63(+). Activated basophil and eosinophil populations were observed using surface markers CD193(+) and CD2P3c(+), respectively, in-tandem with CD63(+). Activated basophils were observed using the surface marker profile of CD63(+)/CD203c(+). Activated eosinophils were observed using the surface marker profile of CD63(+)/CD193(+)/CD203c(+). Activated neutrophils were observed using the surface marker profile of CD63(+)/CD193(−)/CD203(−). Activation of subpopulations was quantified as fold-change compared to the mean of the negative control samples.

A BD Accuri™ C6 Flow Cytometer was used for analysis, featuring four fluorescent channels used for rhodamine 123 (RH 123) (green fluorescent channel) and three other surface/activation markers (e.g., CD33, CD16, CD63).

Exemplary antibodies used throughout the Examples are shown in Table 1.

TABLE 1
Exemplary Antibodies

| Antibody | Conjugated | Excitation | | Emission | | |
|---|---|---|---|---|---|---|
| Target | Fluorophore | wavelength | | wavelength | | Manufacturer/Product |
| CD16 | PE | 488/566 | nm | 578 | nm | Invitrogen, CD16 Antibody, |
| | | | | | | PE (12-0168-42) |
| CD33 | PE-Cy7 | 488 | nm | 779 | nm | BD, PE-Cy7 Mouse Anti- |
| | | | | | | Human CD33 |
| CD193 | Perp-Cy5.5 | 488 | nm | 695 | nm | BD, CD193 Mouse anti- |
| | | | | | | Human, PerCP-Cy5.5, Clone: |
| | | | | | | 5E8 (also known as 5E8-G9- |
| | | | | | | B4) |
| CD123 | APC | 650 | nm | 660 | nm | Invitrogen, CD123 Antibody, |
| | | | | | | APC (17-1239-42) |
| CD63 | PE | 488/566 | nm | 578 | nm | Invitrogen, CD63 Antibody, |
| | | | | | | PE (12-0639-42) |
| Citrullinated | n/a | | | | | Cayman, Histone H3 |
| Histone H3 | | | | | | (Citrullinated R2 + R8 + R17) |
| (mouse) | | | | | | Monoclonal Antibody |
| Goat anti- | APC | 650 | nm | 660 | nm | Cayman, Goat Anti-Mouse |
| mouse IgG 2^{nd} | | | | | | IgG: SureLight ™ APC |
| antibody | | | | | | |

### 5.1.2 Validation of DHR 123 Flow Cytometry Determination of Leukocyte Activation

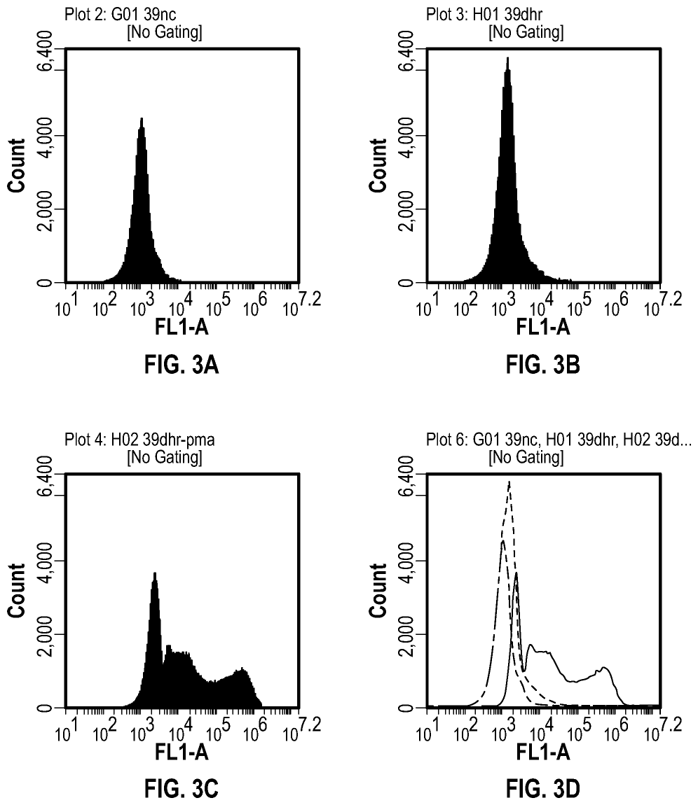

A sample of whole blood was treated with DHIR 123 or mock at 37° C. for 15 min, then treated with phorbol 12-myristate 13-acetate (PMA) and incubated for 25 min. Red and white blood cells were lysed and fixed as described above. Flow cytometry results are shown in FIGS. 3A-3D for negative control (FIG. 3A), DHR 123 only treated control (FIG. 3B), DHR 123+PMA treated (FIG. 3C), as well as an overlap plot of each (FIG. 3D). As shown in FIG. 3C and the right-hand side of FIG. 3D, white blood cells responded to stimulation with PMA and generated reactive oxygen species (ROS) that oxidized DHR 123 to rhodamine 123 (RH 123), as observed by the emission of strong green fluorescence in channel 1 (FL1-A) of the flow cytometer.

### 5.1.3 Validation of Granulocyte Activation Surface Markers for DHR 123 Flow Cytometry Detection of Response to Exogenous Stimulant

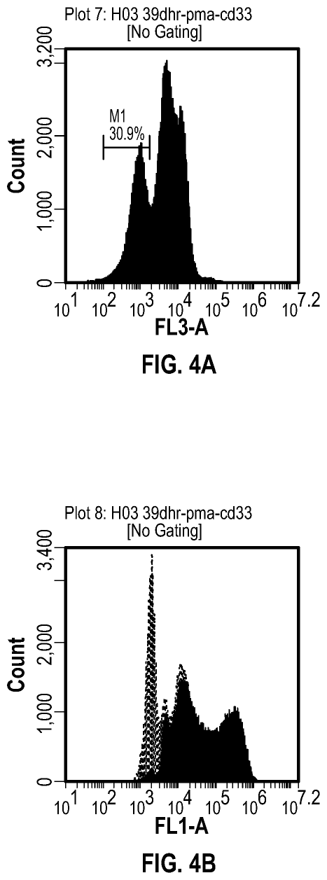

A sample of human whole blood was treated with DHR 123 and/or PMA as above, lysed, and then stained with a phycoerythrin-Cy7 fluorophore-conjugated anti-CD33 antibody (CD33-PE-Cy7) to distinguish the population of lymphocytes from monocytes/granulocytes. FIGS. 4A-4B show histogram plots of fluorescence observed for CD33 (channel 3; FL3-A) and RH 123 (channel 1; FL1-A), respectively. In FIG. 4A, the CD33(−) lymphocyte population appears as the left peak (M1, 30.9%). By using this gating strategy, the majority of cells in the M1 region were lymphocytes. The corresponding peak labeled with (gray) color is shown in FIG. 4B for RH 123 fluorescence intensity (channel 1; FL1-A). In FIG. 4B, the RH 123 green fluorescent signal intensity spanned from about 10⁴ to about 10⁶, while the averaged negative control intensity was about 2×10³. Together, FIGS. 4A-4B show that lymphocytes were not stimulated by PMA, but that other cells such as monocytes and granulocytes were responding to PMA stimulation to generate strong RH 123 fluorescent signals.

### 5.1.4 Time Course Study of DHR 123 Flow Cytometry Blood Test

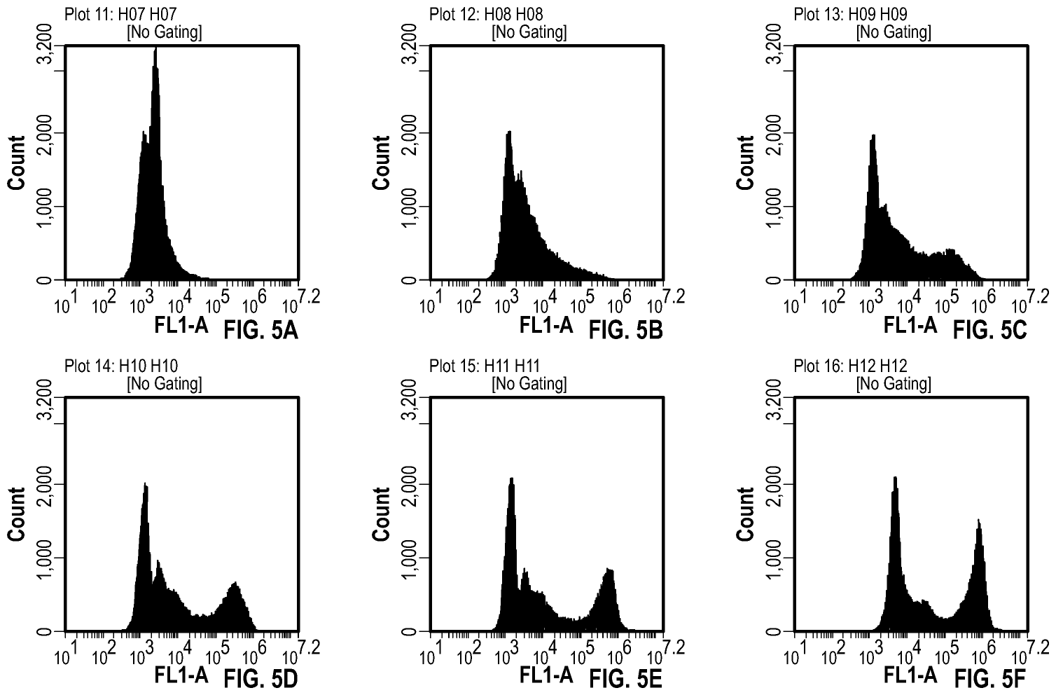

A time course study was performed on white blood cells following treatment with DHR 123 and PMA stimulant. RH 123 fluorescence increased according to the time of incubation with PMA (FIGS. 5A-5F). FIG. 5A represents the negative control of cells loaded with DHR 123 only, while FIG. 5B-5F represent cells treated with DHR 123 and stimulated with PMA for 15, 20, 25, 30, and 40 minutes, respectively. Since DHR 123 is not auto-fluorescent, only cells that produce ROS will oxidize DHR 123 into fluorescent RH 123 for signal emission, especially ROS-producing granulocytes. After 40-minute stimulation of PMA (FIG. 5F), a strong peak having an average intensity of about 10⁶ unit was observed, which was almost 500 times the average intensity for negative control (FIG. 5A).

### 5.1.5 Exemplary Protocol for DHR 123 and Cell Surface Marker Staining for DHR 123 Flow Cytometry Blood Test

Provided in FIG. 1 and FIG. 2 are exemplary protocols.

Provided below is an exemplary protocol for DHR 123 treatment and cell surface marker staining for DHR 123 flow cytometry blood test.
1. Obtain a blood sample from a subject; e.g.,
   1 mL blood sample for each person was obtained.
2. Treat with DHR 123 and/or stimulant (e.g., PMA); e.g.,
   100 μL whole blood was used for each group: 1) cell only control; 2) DHR 123 loaded cells; 3) DHR 123 loaded cells stimulated with stimulant (e.g., PMA). The suggested DHR 123 final concentration was 1˜5 ug/mL, and PMA concentration was 0.3˜3 μg/mL.
3. Fix and lyse the cells; e.g.,
   After stimulant treatment, the cells were fixed and red blood cells were concurrently lysed by adding lysis/fixation solution (e.g., 850 μL (800 μl PBS, 25 μL lyse solution, 25 L 37% formaldehyde)) and incubating 5 min at room temperature.
4. Prepare cells for staining; e.g.,
   Cells were centrifuged and the supernatant discarded;
   Formaldehyde solution (e.g., 500 μL, 3.7% formaldehyde) was added and incubated for 5 more minutes for fixation;
   Cells were centrifuged and the supernatant discarded;
   Cells were washed with 350 μL PBS twice, and then centrifuged before discarding the supernatant;
   Cells were centrifuged and the supernatant discarded;
5. Stain the cells; e.g.,
   80 μL staining buffer (PBS+0.1% albumin+0.01% tween 20) was added, along with 5 μL of antibody solution for CD16, CD123 and CD193 and the sample incubated for 30 min at room temperature;
6. Prepare cells for flow cytometry; e.g.,
   Cells were washed with PBS, centrifuged and the supernatant discarded;
   Cells were resuspended in 125 μL PBS for subsequent flow cytometer analysis.

### 5.1.6 Exemplary Mathematical Compensation Matrix for R123, CD16-PE, CD123-APC, and CD-193-Perp-Cy5.5 Flow Cytometry

A mathematical compensation model was developed by manual adjustment to address potential spectral overlap between fluorophores on the flow cytometer (BD Accuri™ C6 Flow Cytometer; see Table 2).

TABLE 2
Compensation matrix for DHR 123 (RH 123),
CD16-PE, CD123-APC, CD-193-Perp-Cy5.5

| FL1 (RH 123) | FL2 | FL3 | FL4 |
|---|---|---|---|
| | (CD16-PE) | (CD123-APC) | (CD-193-Perp- |
| | | | Cy5.5) |
| | 5.03 | 0.1 | 0.46 |
| FL2 (CD16-PE) | FL1 | FL3 | FL4 |
| | (RH 123) | (CD123-APC) | (CD-193-Perp- |
| | | | Cy5.5) |
| | 17.66 | 0.03 | 0.16 |
| FL3 (CD123-APC) | FL1 | FL2 | FL4 |
| | (RH 123) | (CD16-PE) | (CD-193-Perp- |
| | | | Cy5.5) |
| | 11.03 | 28.67 | 1.84 |
| FL4 (CD-193-Perp- | FL1 | FL2 | FL3 |
| Cy5.5) | (RH 123) | (CD16-PE) | (CD123-APC) |
| | 0.28 | 0.56 | 8.61 |

### 5.1.7 Granulocyte Cell Type Identification Studies

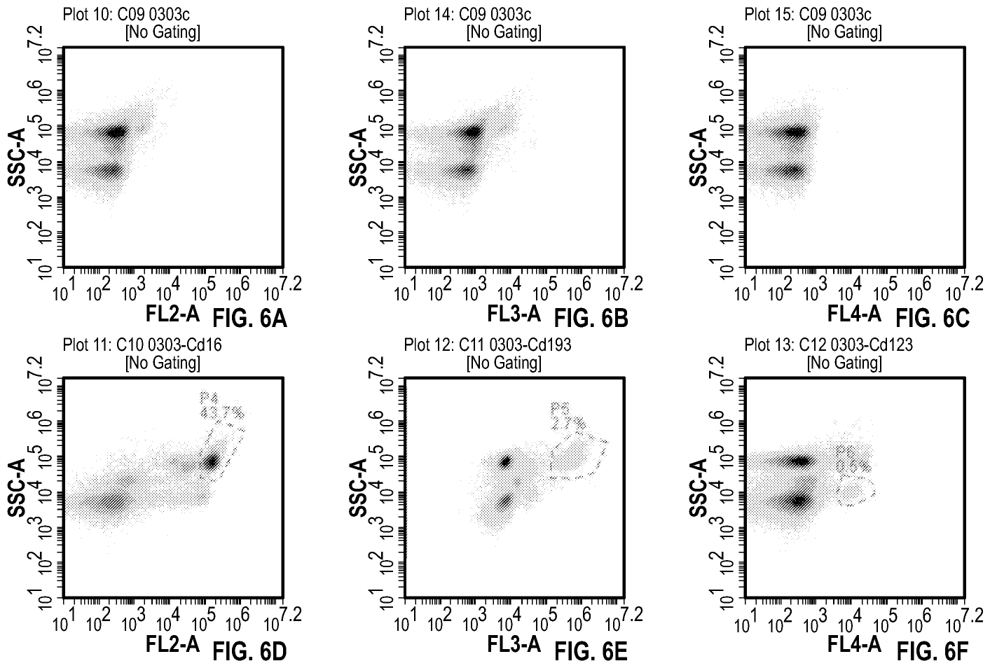

Flow cytometry studies were designed to identify granulocyte subpopulations (e.g., neutrophils, eosinophils, basophils) from blood samples based on surface marker positivity or negativity, side scattering properties, and apparent total population percentage. FIGS. 6A-6F show single color staining (FIG. 6D: CD16-PE, channel 2, FL2-A; FIG. 6E: CD-193-Perp-Cy5.5, channel 3, FL3-A; FIG. 6F: CD-123-APC, channel 4, FL4-A) versus cells-only negative controls (FIGS. 6A-6C, respective to FIGS. 6D-6F). From CD16-PE staining (FIG. 6D) a selected population labeled P4 appeared as likely neutrophils (e.g., CD16(+), high SSC, and apparent total population of ˜43.7% of the whole leukocytes). From CD-193-Perp-Cy5.5 staining (FIG. 6E) a selected population labeled P5 appeared as likely eosinophils (e.g., CD193(+), high SSC, and apparent total population of ˜2.7% of the whole leukocytes). From CD-123-APC staining (FIG. 6F) a selected population labeled P6 appeared as likely basophils (e.g., CD123(+), low SSC, and apparent total population of ˜0.5% of the whole leukocytes).

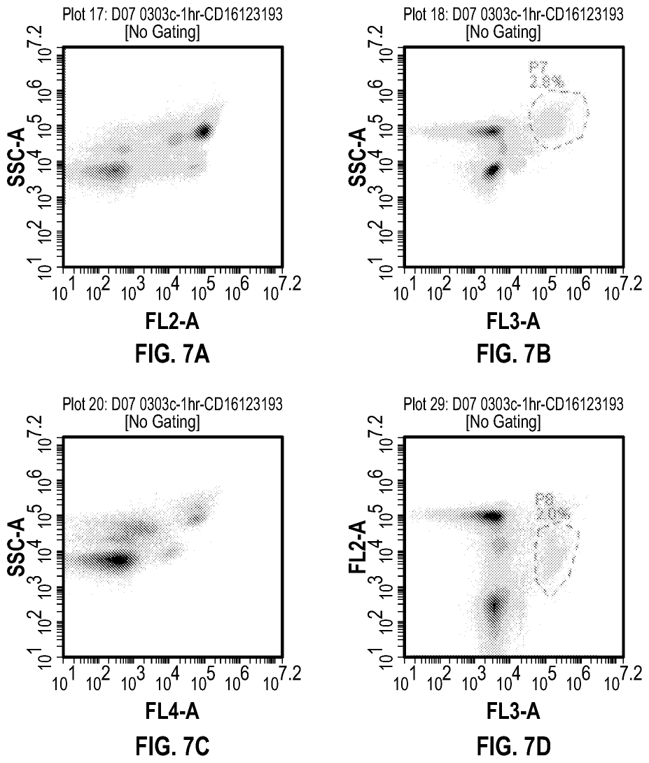

Combinations of surface markers CD16, CD123, CD193 were investigated to improve identification of granulocyte subpopulations. For example, in FIG. 7B, cells positive for CD-193-Perp-Cy5.5 staining appeared as eosinophils and accounted for 2.8% (P7) of total white blood cells. Further analysis combining CD16 and CD193 staining (FIG. 7D) showed an apparent population of eosinophils (CD16− and CD193+) was 2.0% (P8) of total white blood cells.

### 5.1.8 Time Course Analysis of Granulocyte Activation and Surface Markers Using DHR 123 Flow Cytometry

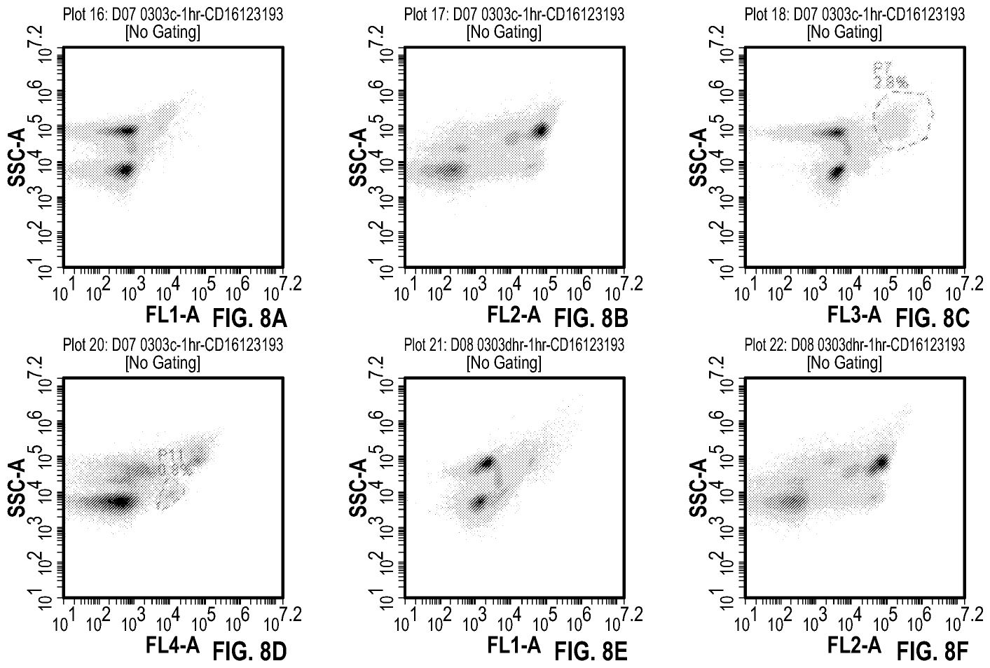

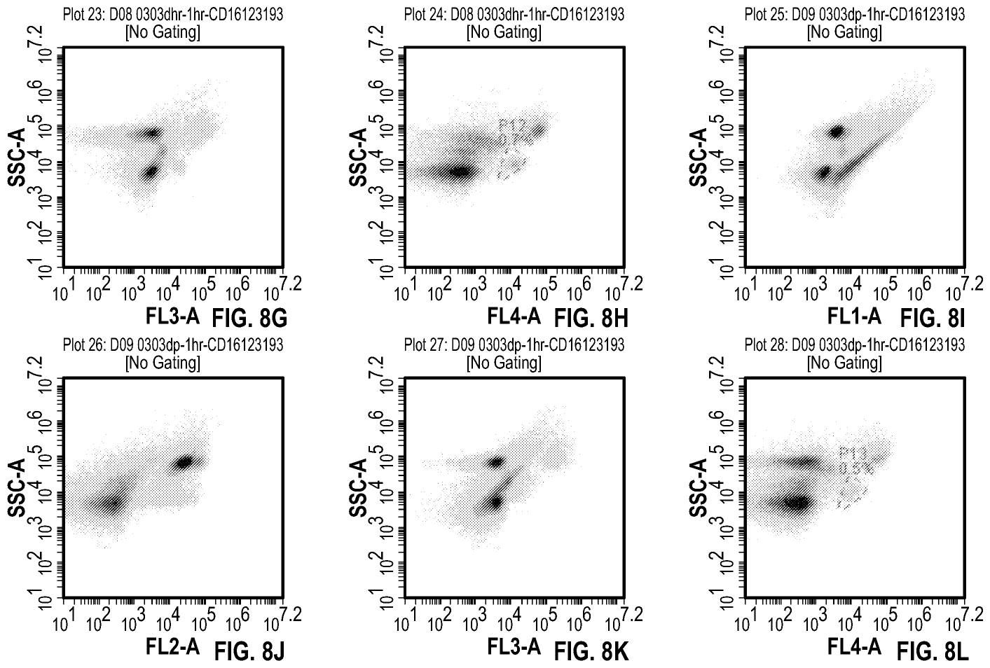

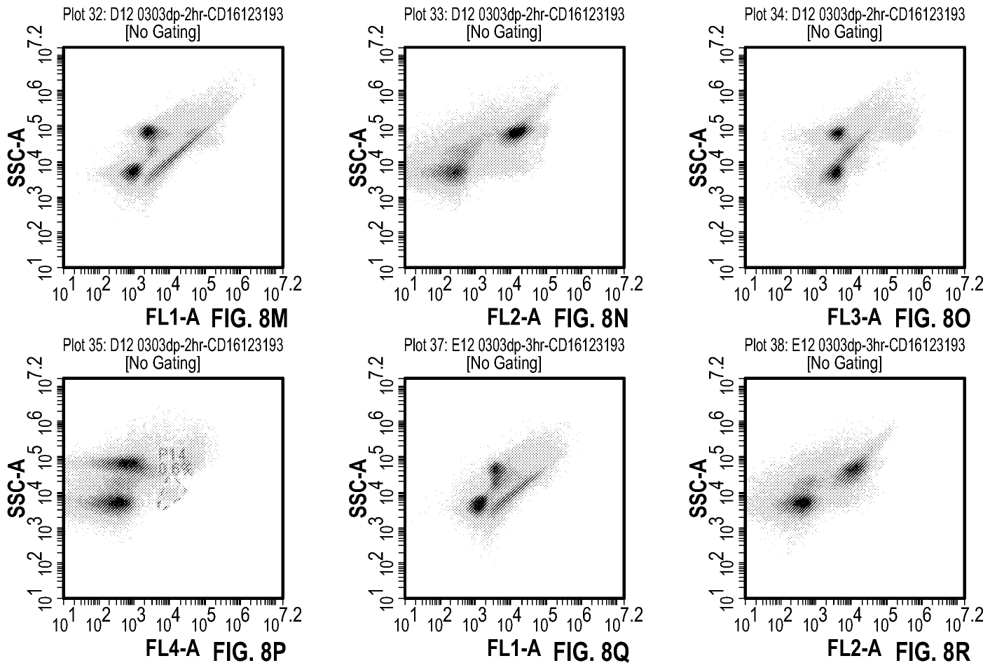

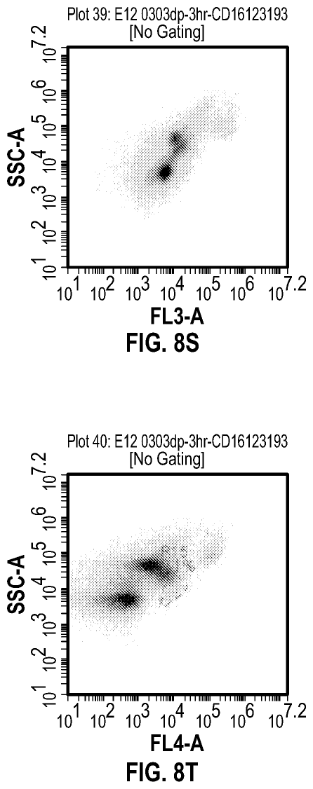

A time course study of R123 and cell surface marker fluorescence was performed over incubation times of 1-3 hrs following treatment with DHR 123 and/or PMA stimulant (FIGS. 8A-8T). Both R123 and cell surface marker fluorescence were affected by incubation time. Eosinophils and basophils showed decreased CD193 (FIGS. 8L, 8P, and 8T) and CD123 (FIGS. 8K, 8O, and 8S) signals over the 1-3 hr incubation time, which without ascribing to any particular theory, was hypothesized as being due to either downregulation of surface marker expression or degradation by degranulation. CD16 fluorescence also exhibited a similar trend of decreased signal intensity over time (FIGS. 8J, 8N, and 8R). It was determined that the best timing for using the DHR 123 chemical dye and these cell surface markers for identification of granulocyte subpopulations and activation was at the onset of degranulation.

### 5.1.9 Summary

Presented are methods for detecting the activation of granulocytes in a biological sample of a subject following exposure to an exogenous stimulant, combining flow cytometry quantification of granulocyte subpopulations and activation by surface markers together with chemical dye-based detection of functional ROS release by granulocytes. Chemical dye DHR 123 was tested for detecting ROS generation in human blood following exposure to PMA stimulant. Other exogenous stimulants or chemical dyes sensitive to ROS are suitable for the same purpose. A preferred flow cytometry start timing was determined to be at the onset of degranulation after exposure to exogenous stimulant. The tested BD Accuri™ C6 Flow Cytometer provided four observable channels, set to observe green fluorescence from oxidation of chemical dye DHR 123 to RH 123 following granulocytic release of ROS after stimulation, as well as three fluorophore-conjugated antibodies specific for surface or activation markers of granulocyte subpopulations (e.g., CD33, CD16 or CD63 markers). The specific combination of surface markers can be adapted for granulocyte subpopulations or when using flow cytometers having additional fluorescent channels.

### 5.2 Example 2: Flow Cytometry Blood Test for Dust Mite, Ragweed Pollen and Food Allergen Stimulants

Cell viability determination using DNA staining was incorporated to improve flow cytometry analysis of granulocyte subpopulations. Propidium iodide (PI) stain was chosen since it does not permeate intact cell membrane, but rather stains cells having damaged cell membranes or extracellular DNAs. Throughout Example 2, additional exogenous stimulants were tested for their ability to instigate granulocyte release of ROS.

### 5.2.1 Time Course Analysis of Granulocyte Reactivity to Dust Mite Stimulant

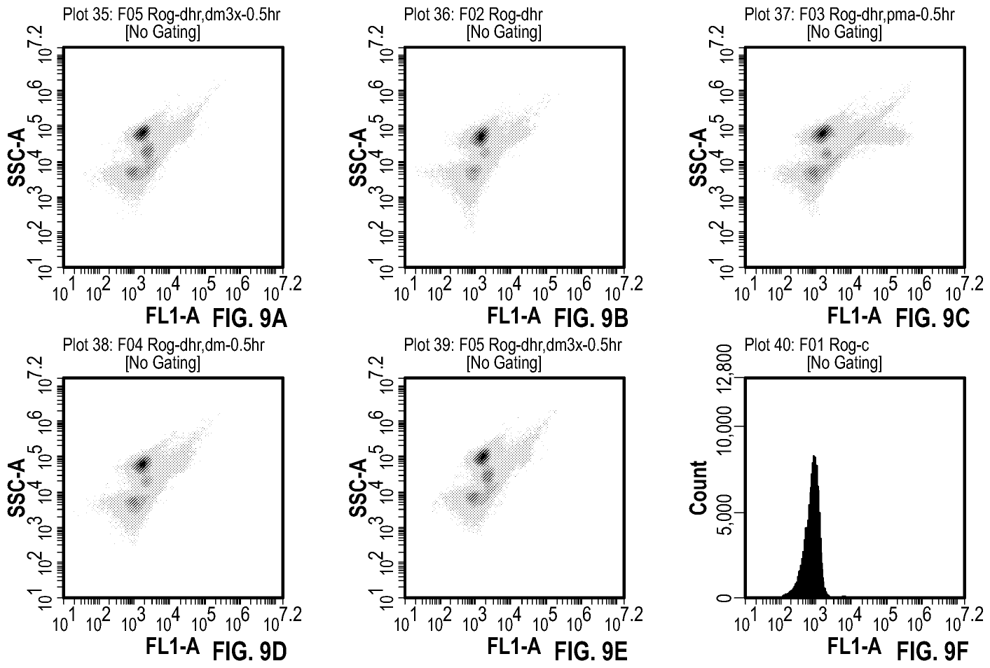

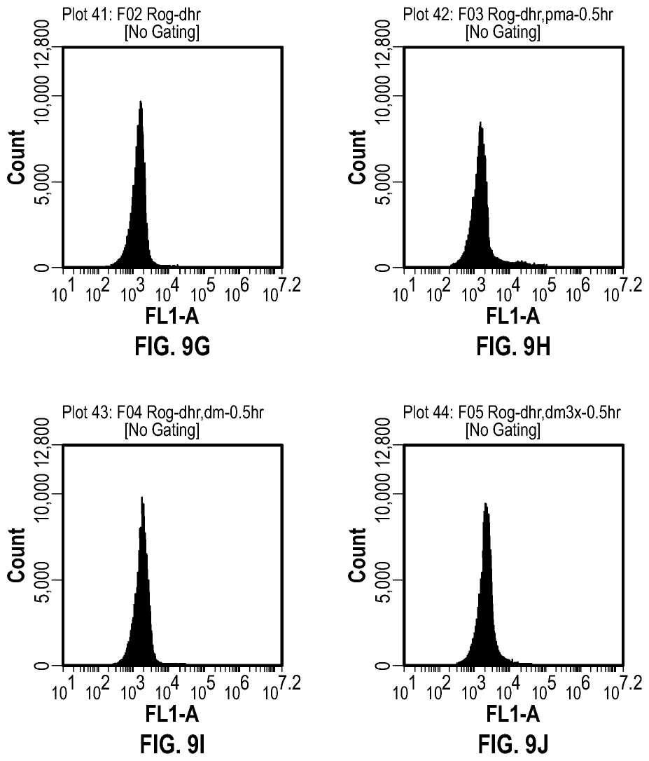

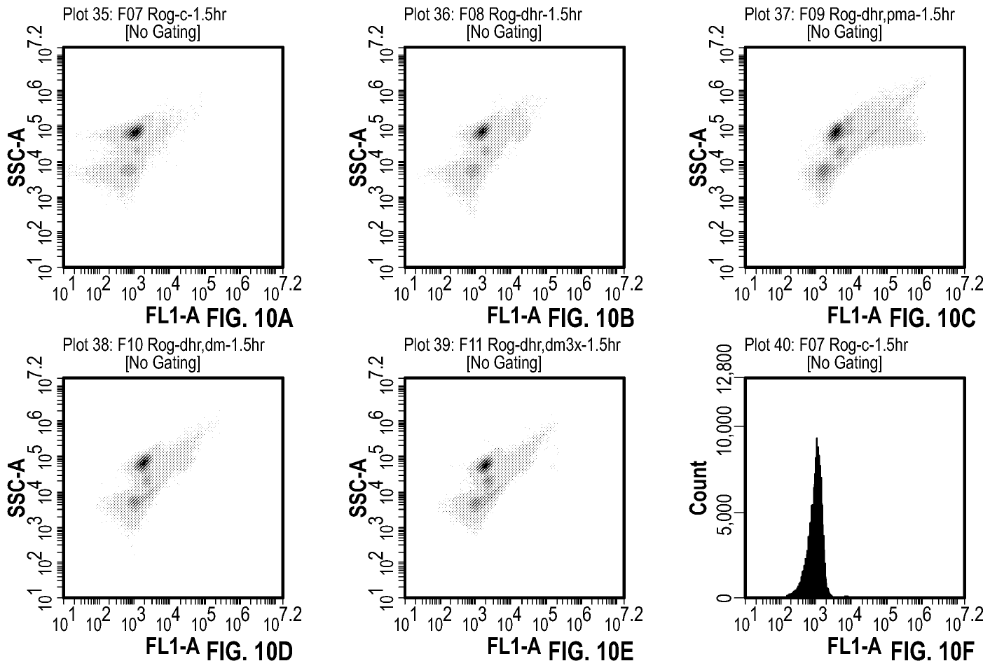

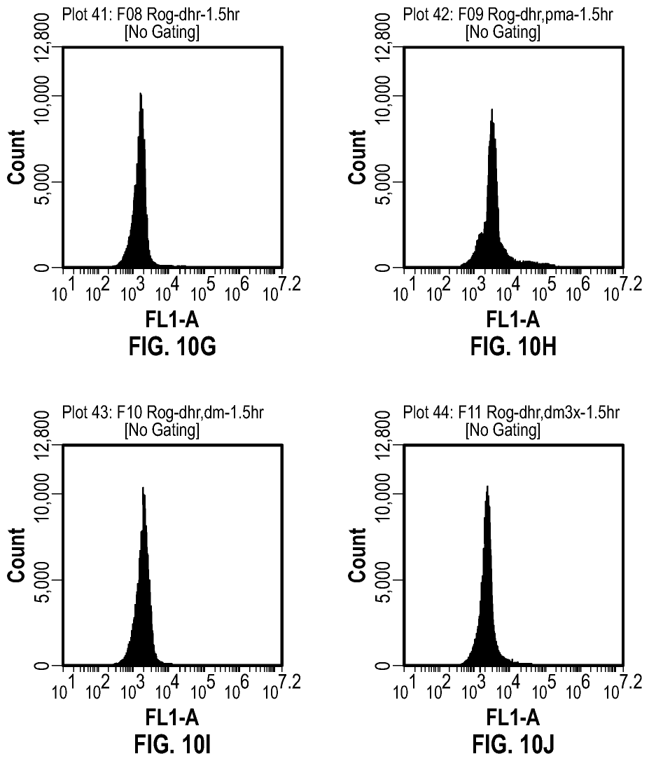

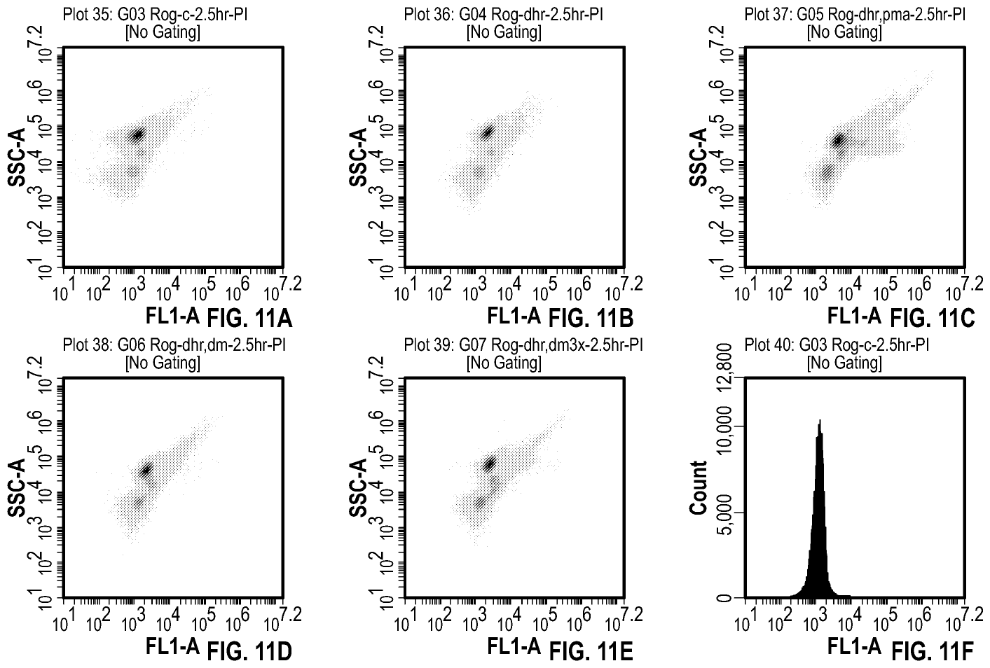

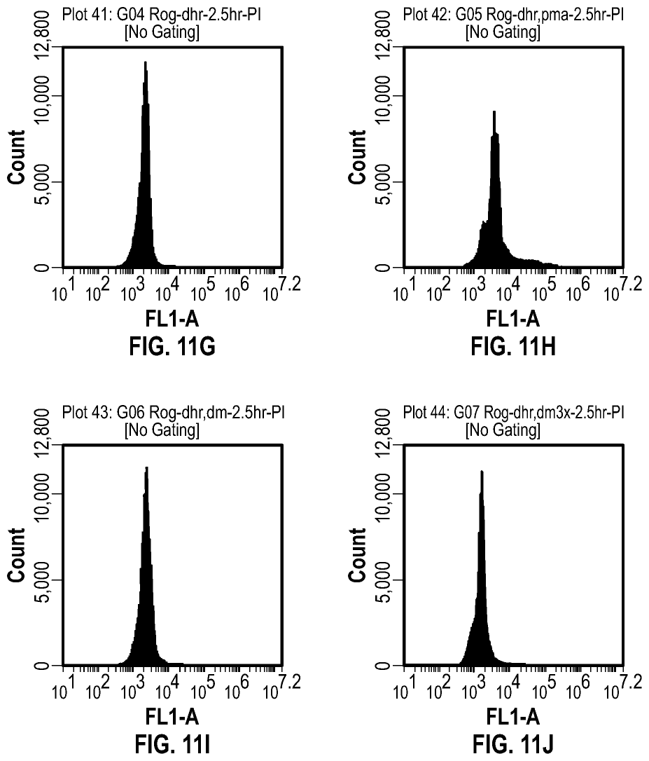

Flow cytometry analysis of human blood samples was performed following exposure to exogenous dust mite (“DM”) stimulant using time course measurements of RH 123 and PI fluorescence. Density plots and histograms were determined for samples containing: control cells only, DHR 123 only, DHR 123+PMA stimulant (positive control), DHR 123+DM stimulant, and DHR 123+3× DM stimulant, following incubation times of 0.5 hours (FIGS. 9A-9J), 1.5 hours (FIGS. 10A-10J), and 2.5 hours (FIGS. 11A-11J), respectively. Propidium iodide staining was used as a measure of cell viability. The mean fluorescent intensity for RH 123 on channel 1 (FL1-A) is shown in Table 3 for each group. As shown in FIGS. 9A-9J, FIGS. 10A-10J, FIGS. 11A-11J, and Table 3, the RH 123 fluorescent signals from dust mite exposed group dust mite increased steadily over 0.5, 1.5 and 2.5 hours. The dust mite exposed group showed higher MFI values than the DHR 123 only treated group, both of which were lower than PMA treated group.

TABLE 3
Mean fluorescent intensity of RH 123 on channel 1 (FL1-A) for dust mite stimulant-
exposed and control groups at 0.5, 1.5 and 2.5 hours, respectively.

| | | Cell | | | |
|---|---|---|---|---|---|
| Cell Sample | MFI (0.5 h) | Sample | MFI (1.5 h) | Cell Sample | MFI (2.5 h) |
| (0.5 h) | FL1-A | (1.5 h) | FL1-A | (2.5 h) | FL1-A |
| Cell only | 858.6 | Cell only | 1,132.35 | Cell only control | 1,321.59 |
| control | | control | | (PI stained) | |
| DHR 123 | 1,701.61 | DHR 123 | 1,967.72 | DHR 123 | 2,262.01 |
| | | | | (PI stained) | |
| DHR 123 + | 5,076.27 | DHR 123 + | 14,093.83 | DHR 123 + | 9,353.98 |
| PMA | | PMA | | PMA | |
| | | | | (PI stained) | |
| DHR 123 + | 2,125.92 | DHR 123 + | 2,590.49 | DHR 123 + DM | 2,940.91 |
| DM | | DM | | (PI stained) | |
| DHR 123 + | 2,574.04 | DHR 123 + | 2,906.33 | DHR 123 + | 3,282.13 |
| DM(3x) | | DM(3x) | | DM(3x) | |
| | | | | (PI stained) | |

### 5.2.2 PI Staining Following Exposure to Dust Mite Stimulant

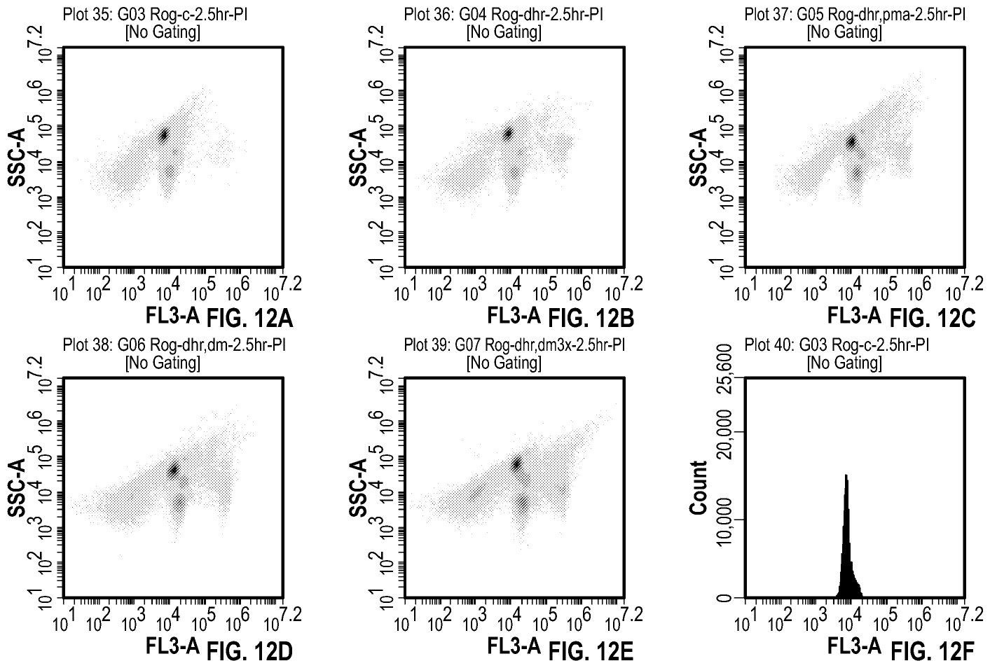

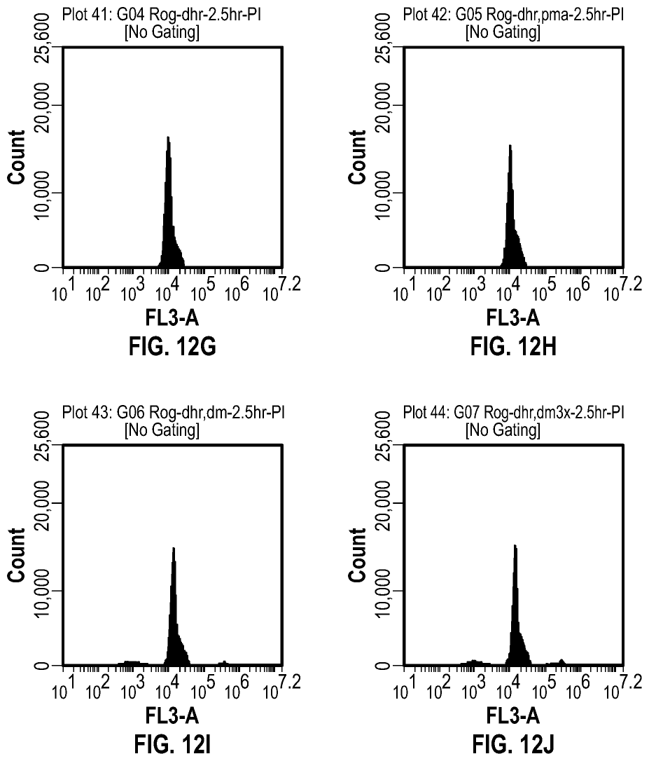

Fluorescence intensity of PI staining was observed on channel 3 (FL3-A) (FIGS. 12A-12J) and summarized in Table 4 below. At 2.5 hours, the groups exposed to dust mite stimulant (FIGS. 12D, 12E and 12I, 12J) exhibited stronger PI stained fluorescent intensity than the DHR 123 and DHR 123+PMA treated group (FIGS. 12C and 12H).

TABLE 4
Mean fluorescent intensity of PI at channel 3 (FL3-A) for dust
mite stimulant-exposed groups and controls at 2.5 hours.

| | MFI (2.5 h) |
|---|---|
| Cell Sample (2.5 h) | FL3-A |
| Control (PI stained) | 8,739.28 |
| DHR 123 (PI stained) | 15,839.23 |
| DHR 123 + PMA (PI stained) | 17,902.33 |
| DHR 123 + DM (PI stained) | 25,385.94 |
| DHR 123 + DM (3x) (PI stained) | 33,723.97 |

### 5.2.3 Time Course Analysis of Granulocyte Reactivity to Ragweed Pollen, Milk, Cucumber, and Basil Stimulants

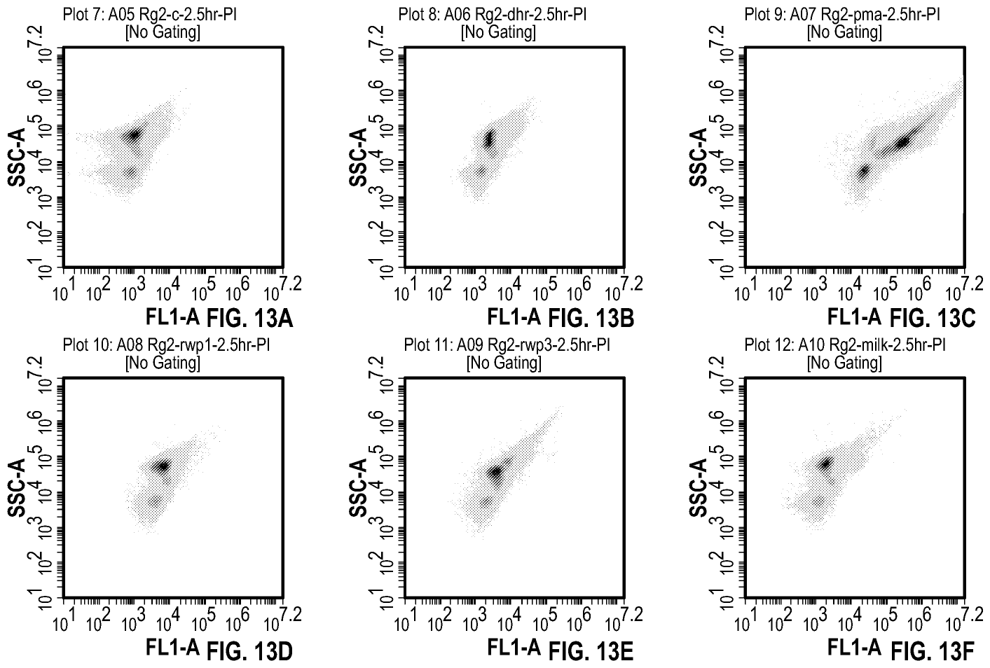

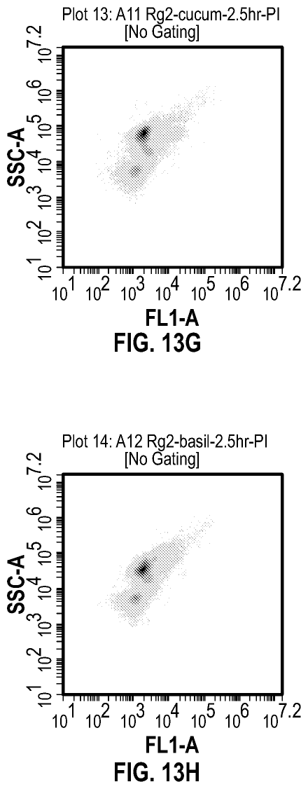

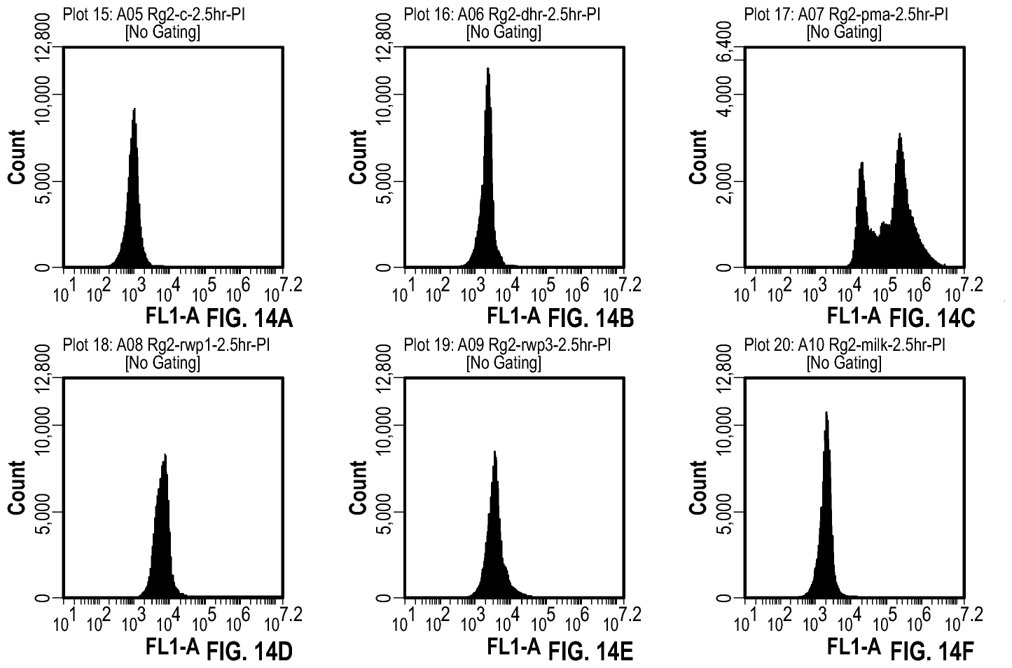

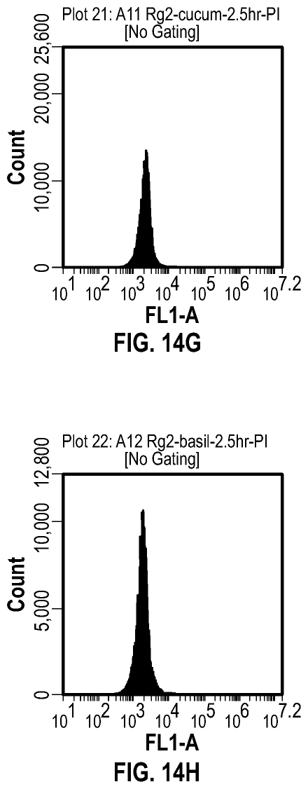

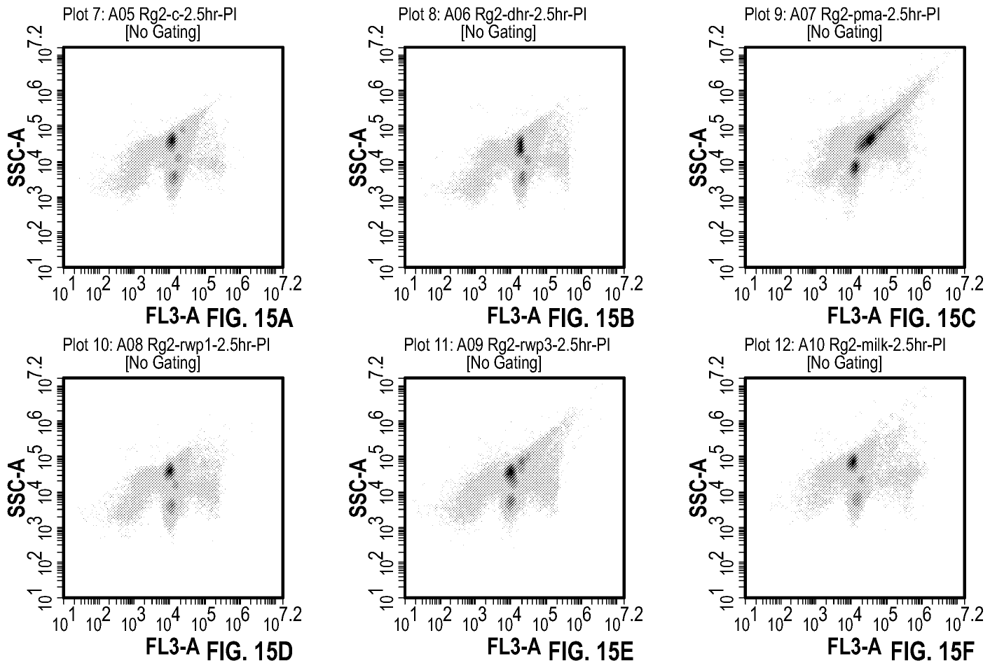

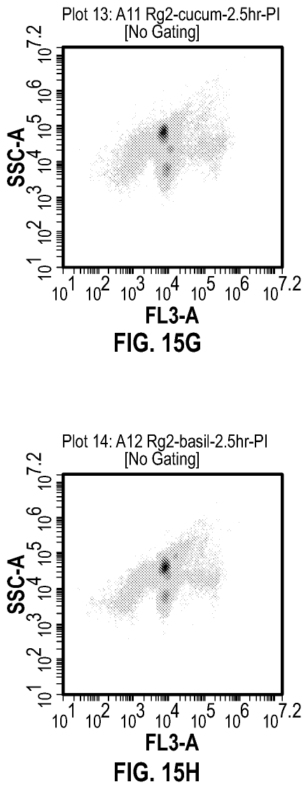

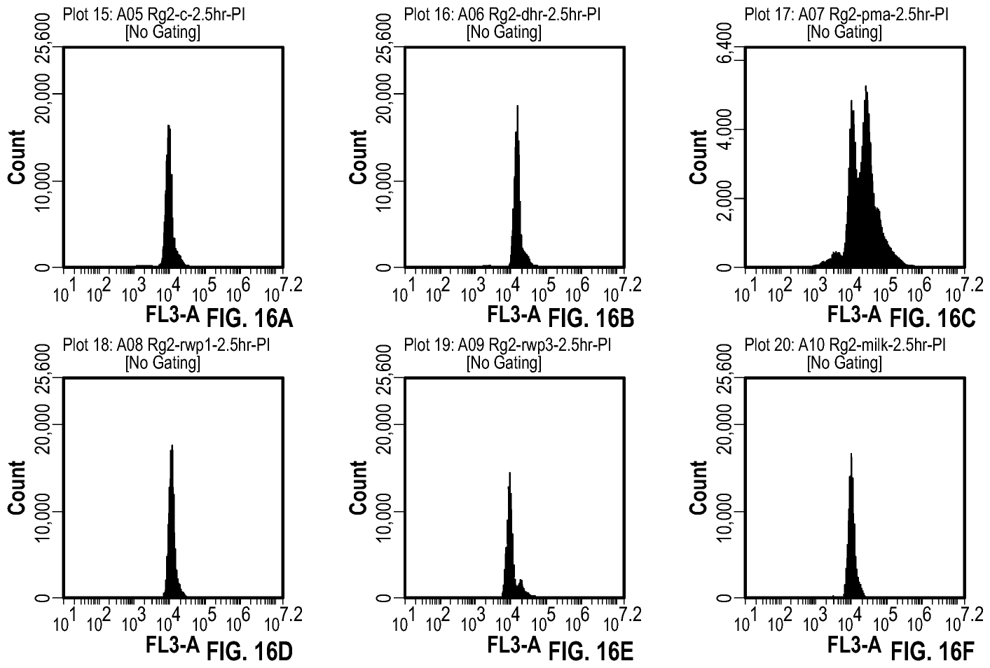

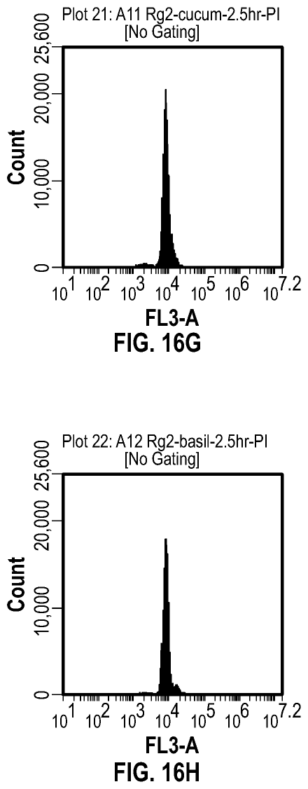

Flow cytometry analysis of human blood samples was performed following exposure to exogenous DM, ragweed pollen (RWP), milk (MLK), cucumber (CUC), and basil (BAS) stimulants using RH 123 and PI fluorescence measurement. Density plots and histograms were determined for samples containing: control cells, DHR 123 only, DHR 123+PMA stimulant, DHR 123+DM stimulant, DHR 123+RWP stimulant (1×: 2.5 μL; 2×: 5.0 μL), DHR 123+MLK stimulant (5 μL), DHR 123+CUC stimulant (5 μL), and DHR 123+BAS stimulant (5 μL), following an incubation times of 0.5, 1.5, and 2.5 hours. See RH 123 (FL1-A) density plots at FIGS. 13A-13H; RH 123 (FL1-A) histograms at FIGS. 14A-14H; propidium iodide (PI) (FL3-A) density plots at FIGS. 15A-15H; PI (FL3-A) histograms at FIGS. 16A-16H). Results are summarized in Table 5 and Table 6.

At 2.5 hours post stimulant exposure ragweed pollen induced ROS generation and exhibited high RH 123 fluorescence intensity (FIG. 13D and FIG. 13E; FIG. 14D and FIG. 14E), compared to cells loaded with DHR 123 without stimulant (e.g., FIG. 131B). See also Table 5.

TABLE 5
Mean fluorescent intensity of R123 (channel 1, FL1-A)
for ragweed pollen, milk, cucumber, and basil stimulant-
exposed and control groups at 0.5, 1.5, and 2.5 hours

| | Mean | | Mean | | |
|---|---|---|---|---|---|
| Cell Sample | FL1-A | Cell Sample | FL1-A | Cell Sample | Mean FL1-A |
| (0.5 h) | (0.5 h) | (1.5 h) | (1.5 h) | (2.5 h) | (2.5 h) |
| Control | 2,757.80 | Control | 1,089.39 | Control | 1,079.37 |
| | | | | (PI stained) | |
| DHR 123 | 3,752.18 | DHR 123 | 7,171.88 | DHR 123 | 2,500.82 |
| | | | | (PI stained) | |
| DHR 123 + | 58,792.96 | DHR 123 + | 168,741.44 | DHR 123 + PMA | 361,510.02 |
| PMA | | PMA | | (PI stained) | |
| DHR 123 + | 3,671.91 | DHR 123 + | 8,111.90 | DHR 123 + RWP1 | 6,455.80 |
| RWP1 | | RWP1 | | (PI stained) | |
| DHR 123 + | 4,813.38 | DHR 123 + | 8,092.98 | DHR 123 + RWP3 | 4,721.42 |
| RWP3 | | RWP3 | | (PI stained) | |
| DHR 123 + | 2,732.32 | DHR 123 + | 4,719.67 | DHR 123 + MLK | 2,153.29 |
| MLK | | MLK | | (PI stained) | |
| DHR 123 + | 2,700.20 | DHR 123 + | 6,696.42 | DHR 123 + CUC | 2,168.11 |
| CUC | | CUC | | (PI stained) | |
| DHR 123 + | 3,176.58 | DHR 123 + | 7,118.33 | DHR 123 + BAS | 2,164.23 |
| BAS | | BAS | | (PI stained) | |

In the PI measurements, ragweed pollen did not induce positive PI signal (FIG. 15D and FIG. 14E; FIG. 16D and FIG. 16E). The food allergens of milk (FIGS. 13F, 14F, 15F, and 16F), cucumber (FIGS. 13G, 14G, 15G, and 16G), and basil (FIGS. 13H, 14H, 15H, and 16H) also did not induce positive PI signal. See Table 6.

TABLE 6
Mean fluorescent intensity of PI (channel 3, FL3-A)
for ragweed pollen, milk, cucumber, and basil stimulant-
exposed groups and controls at 2.5 hours.

| | Mean FL3-A |
|---|---|
| Cell Sample (2.5 h) | (2.5 h) |
| Control (PI stained) | 11,530.43 |
| DHR 123 (PI stained) | 18,122.64 |
| DHR 123 + PMA (PI stained) | 34,645.62 |
| DHR 123 + RWP1 (PI stained) | 13,225.49 |
| DHR 123 + RWP3 (PI stained) | 15,654.51 |
| DHR 123 + MLK (PI stained) | 13,763.90 |
| DHR 123 + CUC (PI stained) | 10,762.16 |
| DHR 123 + BAS (PI stained) | 10,074.96 |

### 5.2.4 DHR 123 and PI Flow Cytometry Blood Testing Using Increased Stimulant Concentration

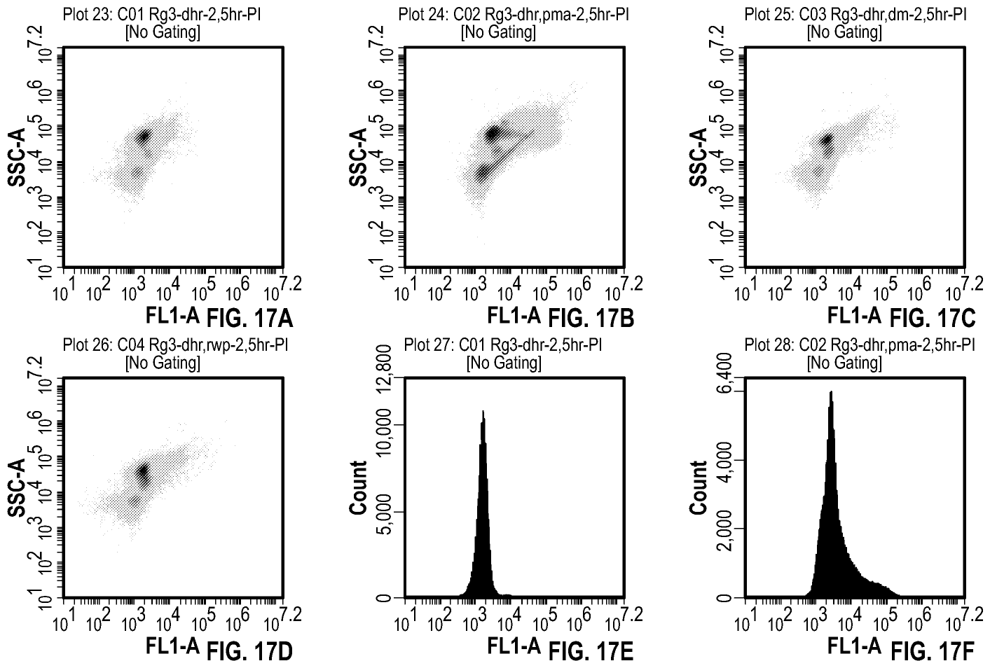

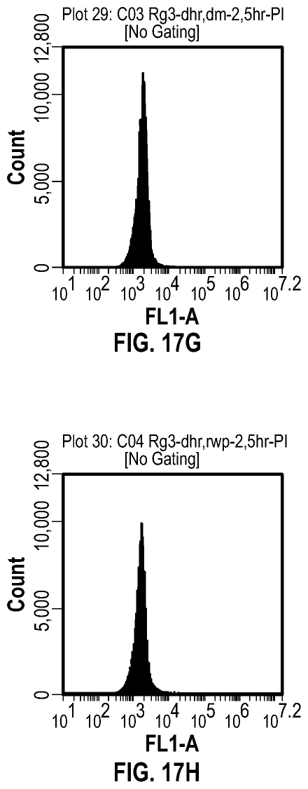

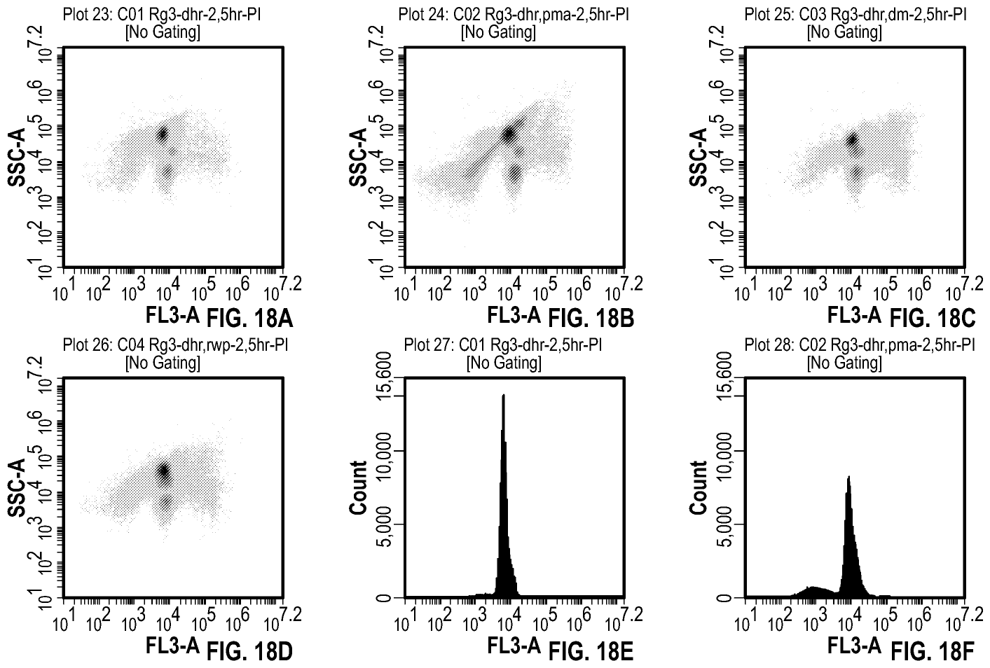

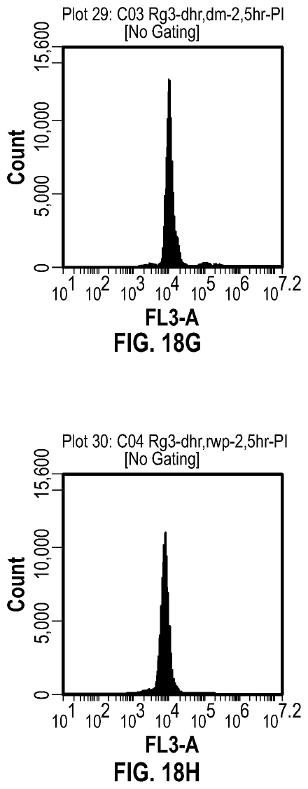

DHR 123 and PI flow cytometry blood testing was repeated using increased amounts of stimulant (8 μL of DM stimulant or RWP stimulant per 100 μL blood) and incubated for 2.5 hours. Density and histogram plots for RH 123 measurements (channel 1, FL1-A) are shown in FIGS. 17A-17H, and summarized in Table 7 below. Density and histogram plots for PI measurements (channel 3, FL3-A) are shown in FIGS. 18A-18H, and summarized in Table 7 below.

TABLE 7
Mean fluorescent intensity of RH 123 (channel 1, FL1-A)
and PI (channel 3, FL3-A) for dust mite and ragweed pollen
stimulant-exposed groups and controls at 2.5 hours.

| | MFI (2.5 h) | MFI (2.5 h) |
|---|---|---|
| Cell Sample (2.5 h) | FL1-A | FL3-A |
| DHR 123 (PI stained) | 1,669.89 | 8,320.84 |
| DHR 123 + PMA (PI stained) | 10,553.44 | 11,914.00 |
| DHR 123 + DM (PI stained) | 2,090.74 | 19,670.61 |
| DHR 123 + RWP (PI stained) | 1,892.22 | 10,776.31 |

### 5.2.5 Study of Cell Surface Markers

Cell surface markers specific for granulocyte populations of neutrophils, eosinophils and basophils were tested for performance in flow cytometry blood testing.

TABLE 8
Compensation matrix for DHR 123 (RH 123),
CD16-PE, CD123-APC, CD193-PerCP-Cy5.5

| FL1 | FL2 | FL3 | FL4 |
|---|---|---|---|
| (RH 123) | 5.03 | 0.1 | 0.46 |
| FL2 | FL1 | FL3 | FL4 |
| (CD16) | 17.1 | 0.03 | 0.16 |
| FL3 | FL1 | FL2 | FL4 |
| (CD123) | 7.19 | 28.67 | 1.84 |
| FL4 | FL1 | FL2 | FL3 |
| (CD193) | 0.20 | 0.56 | 8.61 |

5.2.5.1 Detection of Neutrophil Activation Following Stimulation with Dust Mite and Ragweed Pollen Stimulants

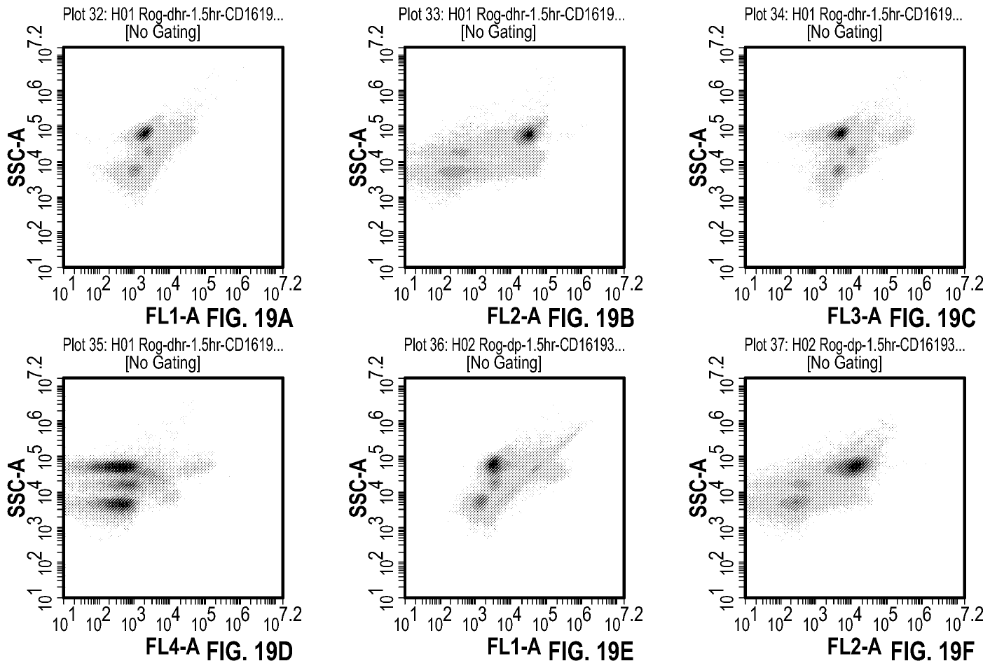

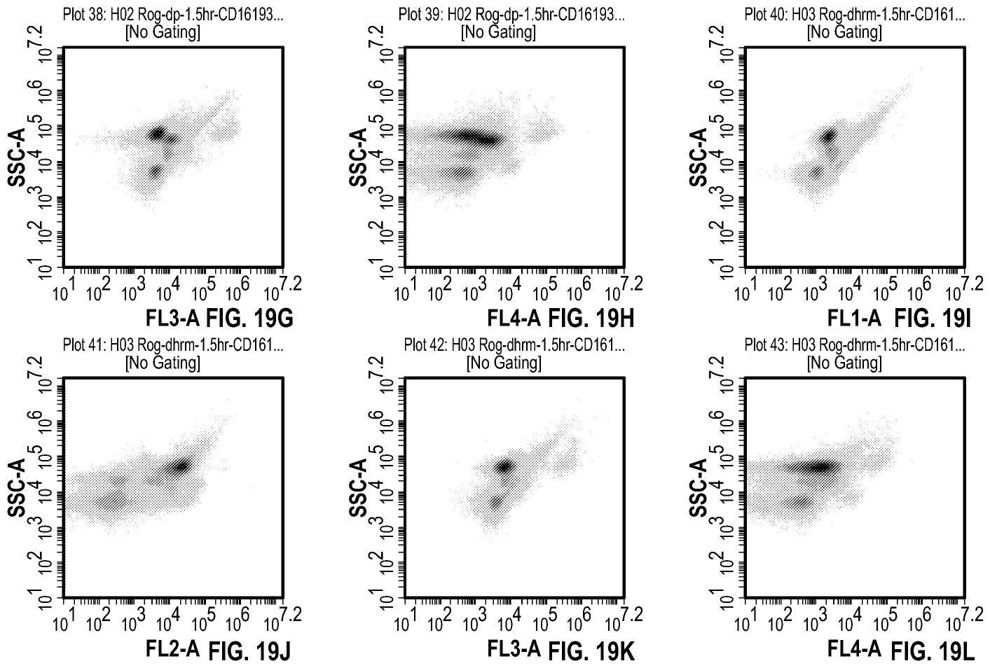

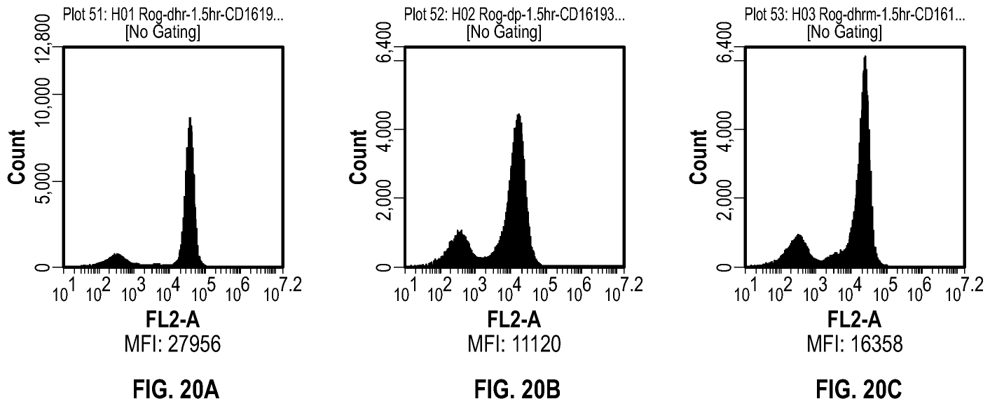

Activation of neutrophils from human blood samples following exposure to exogenous stimulants was investigated using flow cytometry analysis of R123 fluorescence, as well as cell surface markers CD16, CD193, and CD123, and side scattering profiles. Density plots for RH 123 (channel 1; FL1-A), CD16 (channel 2; FL2-A), CD193 (channel 3, FL3-A), and CD123 (channel 4, FL4-A) following exposure to dust mite stimulant for 1.5 hours are shown in FIGS. 19A-19L (DHR 123 only: FIGS. 19A-19D; DHR 123+PMA stimulant: FIGS. 19E-19H; and DHR 123+DM stimulant: FIGS. 19I-19L). Density plots FIGS. 19A, 19E, and 19I indicated that RH 123 signals in FL1-A were higher in the DHR 123+PMA stimulant and DHR 123+DM stimulant exposed groups. Histograms of CD16 fluorescence at 1.5 hours after stimulant exposure are shown in FIGS. 20A-20C (DHR 123 control: FIG. 20A, MFI=27956; DHR 123+PMA stimulant: FIG. 20B, MFI=11120; and DHR 123+DM stimulant: FIG. 20C, MFI=16358). When viewing the density plots of FIGS. 19B, 19F, and 19J and corresponding histograms (FIGS. 20A-20C), the expression of CD16 surface marker was found to be greatly reduced in DHR 123+PMA stimulant and DHR 123+DM stimulant-exposed groups. This result is similar to literature findings that the expression of CD16 surface marker on activated neutrophils decreases (Mol et al., Int. J. Mol. Sci. 2021).

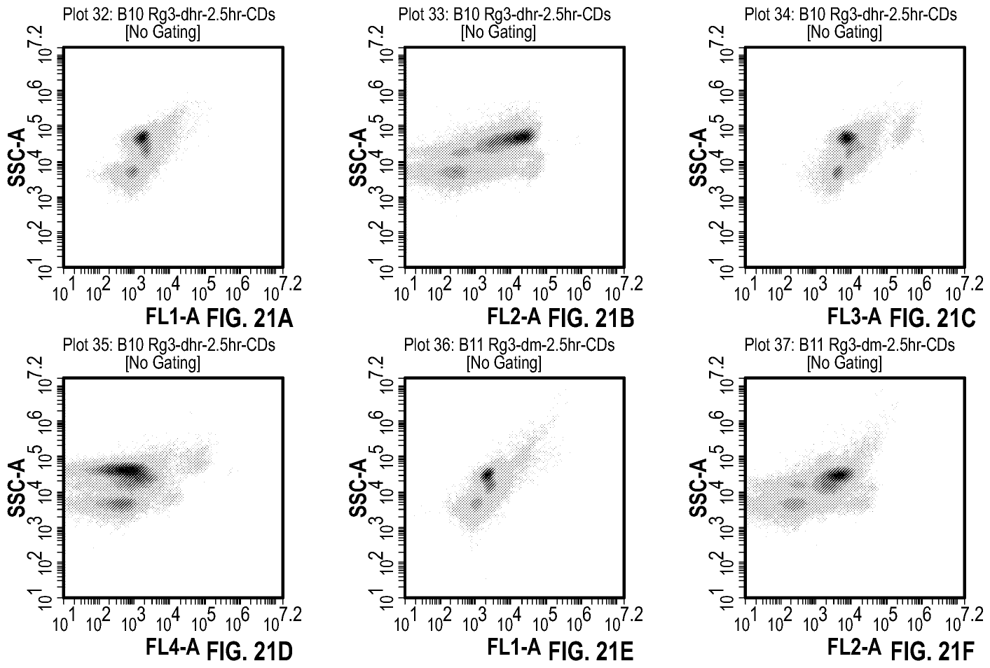

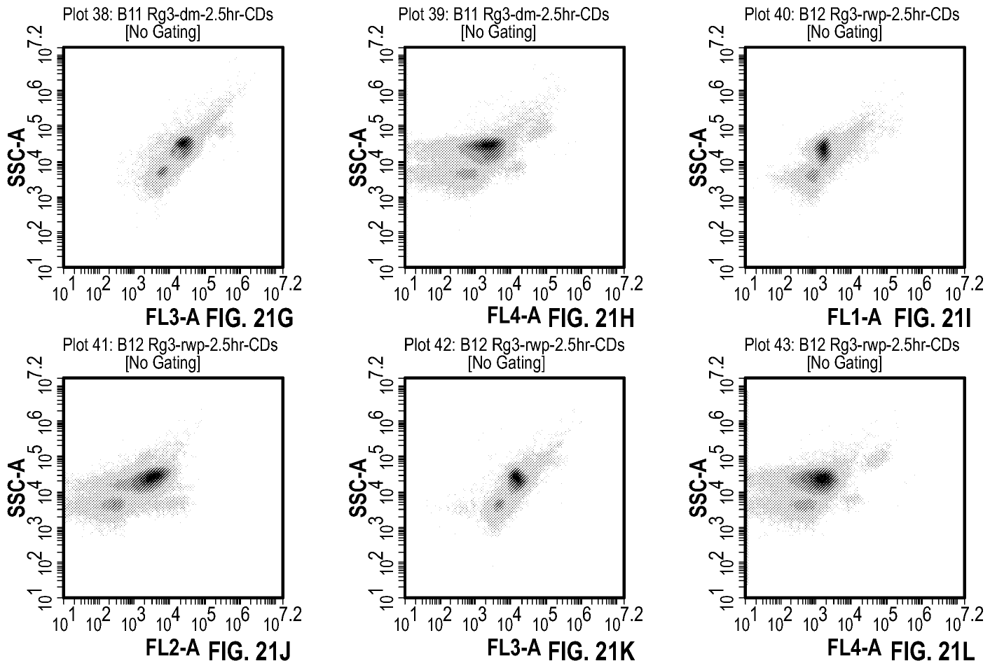

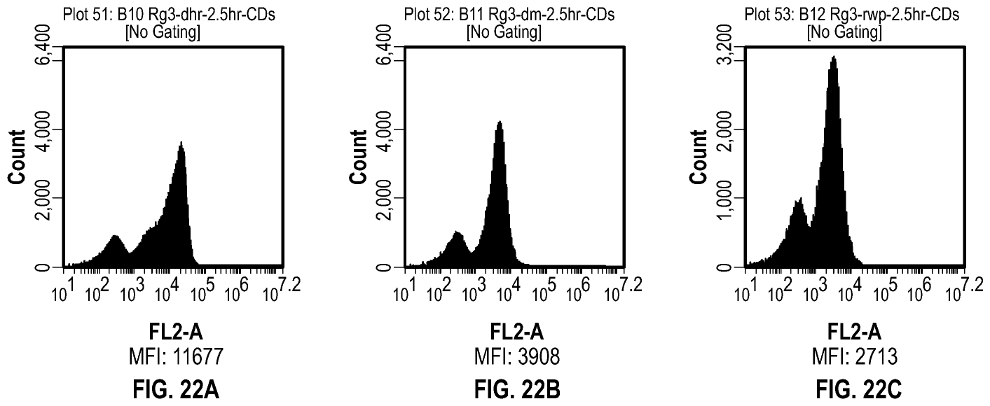

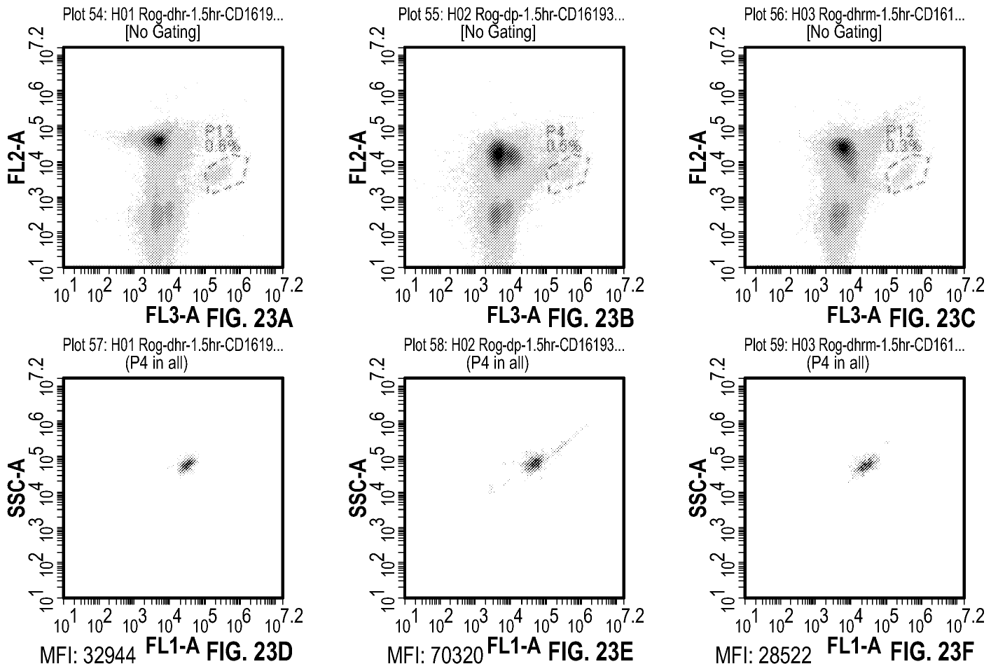

Density plots for DHR 123 flow cytometry blood testing following exposure to following dust mite and ragweed stimulants for 2.5 hours using RH 123 (channel 1; FL1-A), CD16 (channel 2; FL2-A), CD193 (channel 3, FL3-A), and CD123 (channel 4, FL4-A) are shown in FIGS. 23A-23L. DHR 123 only: FIGS. 21A-21D; DHR 123+DM stimulant: FIGS. 19E-19H; and DHR 123+RWP stimulant (FIGS. 19I-19L). Histograms for CD16 (at 1.5 hours) are shown in FIGS. 22A-22C (DHR 123 only: FIG. 22A, MFI=11677; DHR 123+DM stimulant: FIG. 22B, MFI=3908; and DHR 123+RWP stimulant: FIG. 22C, MFI=2713).

A similar trend of decreasing CD16 expression was observed following exposure to stimulant for 2.5 hours (FIGS. 21B, 21F, 21J and FIGS. 22A-22C), which showed that CD16 expression was further reduced along with longer (2.5 hr) exposure to stimulant. It was concluded that neutrophils are activated by dust mite and ragweed pollen stimulants according to this method, with ragweed pollen acting as a stronger activator of neutrophils than dust mite.

### 5.2.5.2 Detection of Eosinophil Activation in Blood Samples by Flow Cytometry

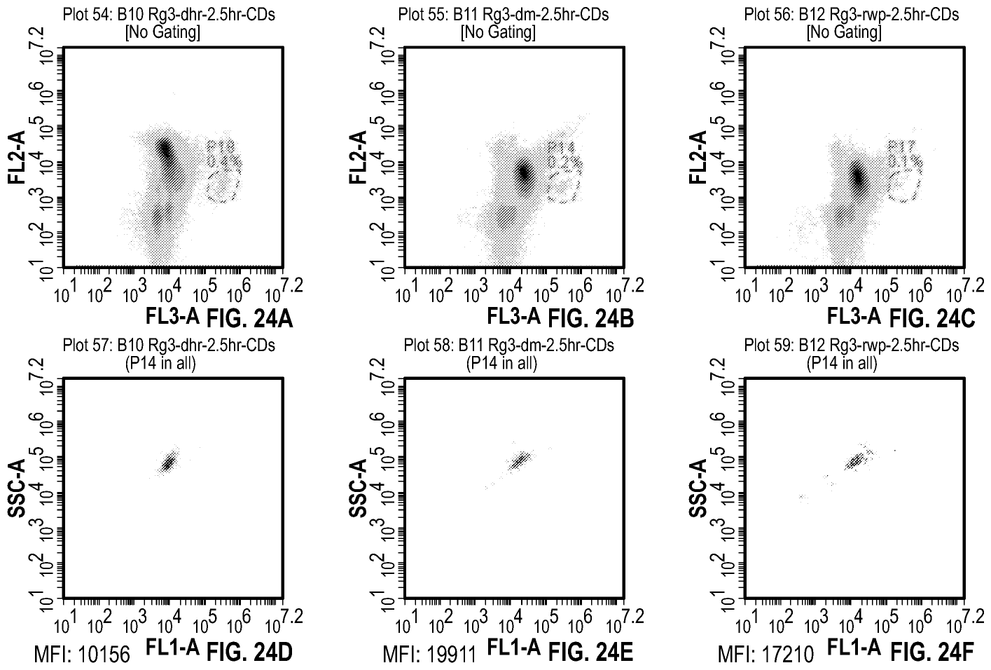

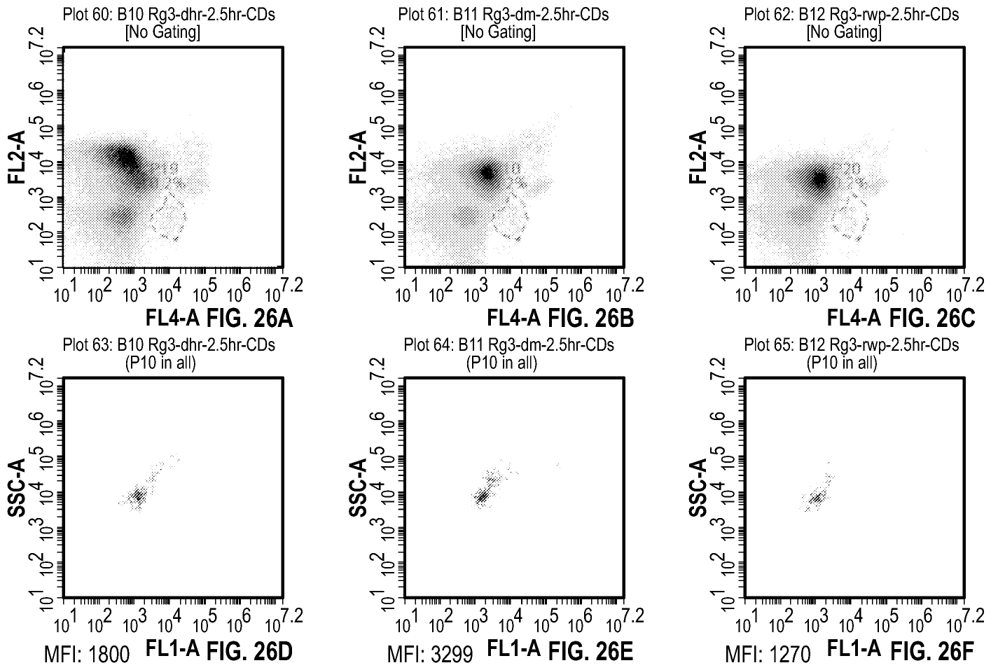

Activation of eosinophils from human blood samples following exposure to exogenous stimulants was investigated using flow cytometry analysis of RH 123 fluorescence, as well as cell surface markers CD16 and CD193, and side scattering profiles. CD193 (CCR3; Chemokine C—C motif receptor 3) is a surface marker expressed in eosinophils and basophils. FIGS. 23A-23C show density plots of CD193 fluorescence signal on channel 3 (FL3-A) for DHR 123 only (FIG. 23A), DHR 123+PMA stimulant (FIG. 23B), and DHR 123+DM stimulant (FIG. 23C) following exposure to stimulant for 1.5 hours. FIGS. 23D-23F show corresponding gated RH 123 results (MFI=32944, MFI=70320, and MFI=28522, respectively). FIGS. 24A-24C show density plots of CD193 fluorescent signals at channel 3 (FL3-A) for DHR 123 only (FIG. 24A), DHR 123+DM stimulant (FIG. 24B), and DHR 123+RWP stimulant (FIG. 24C) following exposure to stimulant for 2.5 hours. FIGS. 24D-24F show corresponding gated RH 123 results (FIG. 24D, DHR 123 only: MFI=10156; FIG. 24E, DHR 123+DM stimulant: MFI=19911, and FIG. 26F, DHR 123+RWP stimulant: MFI=17210).

Eosinophil population was selected using CD193(+) and CD16(−) expression profiles (shown in FIGS. 23A-23C and FIGS. 23A-23C as encircled). Eosinophil population was reduced in PMA stimulant, DM stimulant and RWP stimulant exposed groups. In addition, DHR 123 signals in gated eosinophils was higher than DHR 123 only control group after 2.5-hour incubation. Thus, it was concluded that eosinophils are activated by dust mite and ragweed pollen stimulants according to this method.

### 5.2.5.3 Detection of Basophil Activation in Blood Samples by Flow Cytometry

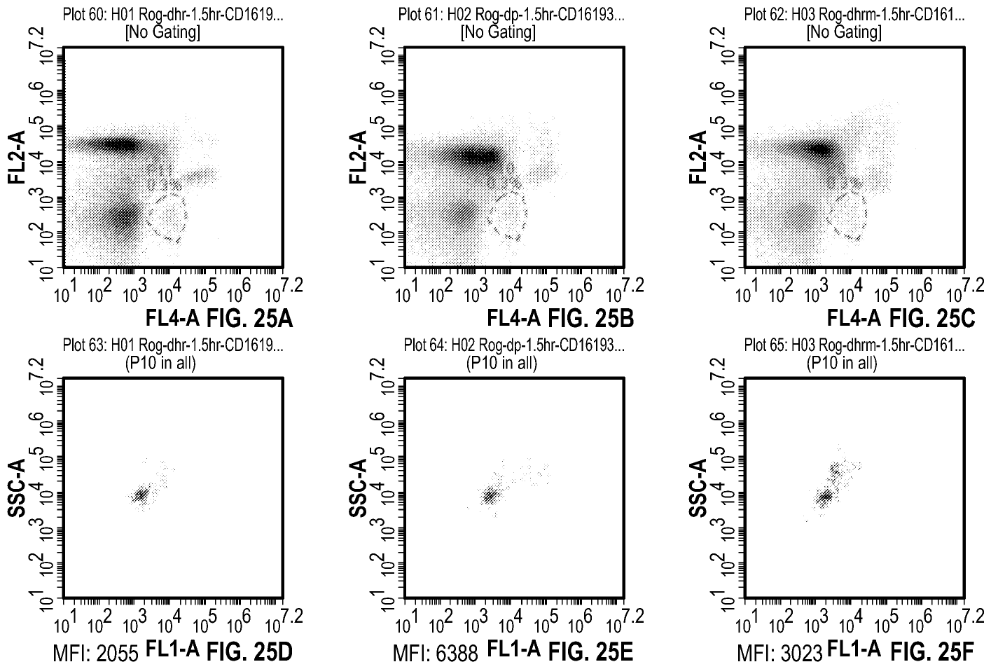

Activation of basophils from human blood samples following exposure to exogenous stimulants was investigated using flow cytometry analysis of RH 123 fluorescence, as well as cell surface markers CD16 and CD193, and side scattering profiles. CD123 is a cell surface marker that is expressed on basophils and eosinophils. FIGS. 25A-25C show density plots of CD123 signals at channel 4 (FL4-A) for DHR 123 only (FIG. 25A), DHR 123+PMA stimulant (FIG. 25B), and DHR 123+DM stimulant exposed group (FIG. 25C) following exposure to stimulant for 1.5 hours. FIGS. 25D-25F show corresponding gated RH 123 results (MFI=2055, MFI=6388, and MFI=3023, respectively). FIGS. 26A-26C show density plots of CD123 signals at channel 4 (FL4-A) for DHR 123 control group (FIG. 26A), DHR 123 and dust mite stimulant-exposed group (FIG. 26B), and DHR 123 and ragweed pollen stimulant-exposed group (FIG. 26C) following exposure to stimulant for 2.5 hours. FIGS. 26D-26F show corresponding gated RH 123 results (MFI=1800, MFI=3299, and MFI=1270, respectively).

Basophil population was selected by having CD123(+)/CD16(−) expression profiles, along with lower side scattering (shown in FIGS. 25A-25C and FIGS. 26A-26C as encircled). In contrast to reducing eosinophil population for different allergen treated groups, basophil population appeared stable over the experiment time period (1.5 hours and 2.5 hours). Only the RWP stimulant-exposed group showed a slight drop on the basophil percentage (0.17% vs. 0.21% for control). It was clear that PMA and DM stimulant induced the activation of basophils because their corresponding RH 123 fluorescent signals were higher than DHR 123 only treated control group. However, RWP stimulant did not induce significant amount of basophil activation. Furthermore, some basophils exhibited high side scattering signals in dust mite stimulant-expose treated group, which could imply the formation of extracellular traps.

### 5.2.6 Summary

Incorporation of cell viability staining enhanced the ability to distinguish granulocyte subpopulations. Cell viability staining was performed using the DNA stain propidium iodide (PI) following exposure to stimulant. Dead cells were gated and removed using the PI(+) viability marker. Other cell viability stains are suitable for the same purpose, for example other fluorescent dyes that are impermeable to intact cell membranes.

Different responses of granulocytes were observed following exposure to different stimuli, with varying reactions to PMA, dust mite and ragweed pollens. PMA induced the activation of all granulocyte populations, as expected. Dust mite simulant successfully activated some of neutrophils, eosinophils and basophils. Ragweed pollen stimulants induced strong activation of neutrophils and some of eosinophils, but was less effective activating basophils.

### 5.3 Example 3: Citrullinated Histone H3 Flow Cytometry Blood Test

Detection of extracellular traps formation was incorporated to improve flow cytometry analysis of granulocyte subpopulations. Citrullinated histone H3 is uniquely generated during neutrophil extracellular trap (NEL) formation and was investigated for its potential to be used as an additional indicator of extracellular trap formation following the positive propidium iodide staining (e.g., as demonstrated in Example 2). This example describes detection of granulocyte activation following exposure of human blood to exogenous stimulant, using dihydrorhodamine 123, propidium iodide, and citrullinated histone H3 staining.

### 5.3.1 Materials and Methods

Peripheral blood samples were collected, diluted, maintained, treated, lysed, and fixed as previously described. Samples were blocked as previously described and incubated with staining buffer containing a 1:400 dilution of Cit-H3 antibody (Cayman Chemical, Michigan, USA) for 30 min at RT. Following two wash steps, the samples were incubated with APC-conjugated secondary (1:200) (Columbia Biosciences, Maryland, USA) for 30 min and subsequent washing steps were performed to remove unbound antibodies. Cells were washed twice and resuspended in PBS prior to analysis using a BD Accuri C6 Flow Cytometer (BD Biosciences). Statistical analysis was performed using the FlowJo v10 (FlowJo). NETs were defined by the in-tandem use of the classis extracellular DNA/viability marker PI(+) and the extracellular trap marker Cit-H3(+). NET formation was quantified as a fold-change relative to the mean of the negative control samples.

A mathematical compensation model was developed by manual adjustment to address potential spectral overlap between fluorophores on the flow cytometer (BD Accuri™ C6 Flow Cytometer; see Table 9).

TABLE 9
Compensation matrix for RH 123, PI, and Citrullinated
Histone H3/secondary antibody conjugated with APC

| FL1 | FL2 | FL3 | FL4 |
|---|---|---|---|
| | 0 | 0.92 | 0.63 |
| FL2 | FL1 | FL3 | FL4 |
| | 17.10 | 12.36 | 0.26 |
| FL3 | FL1 | FL2 | FL4 |
| | 7.19 | 0 | 3.66 |
| FL4 | FL1 | FL2 | FL3 |
| | 0.20 | 0 | 1.09 |

### 5.3.2 Citrullinated Histone H3 Flow Cytometry Blood Testing of Reactivity to Dust Mite and Ragweed Pollen Stimulants

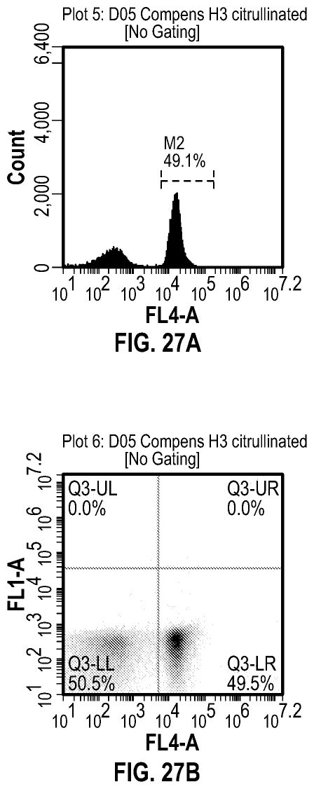

Histogram and density plots of compensation beads with primary (mouse anti-human citrullinated histone H3) and secondary antibodies (goat anti-mouse APC conjugated) are shown in FIG. 27A and FIG. 27B, respectively.

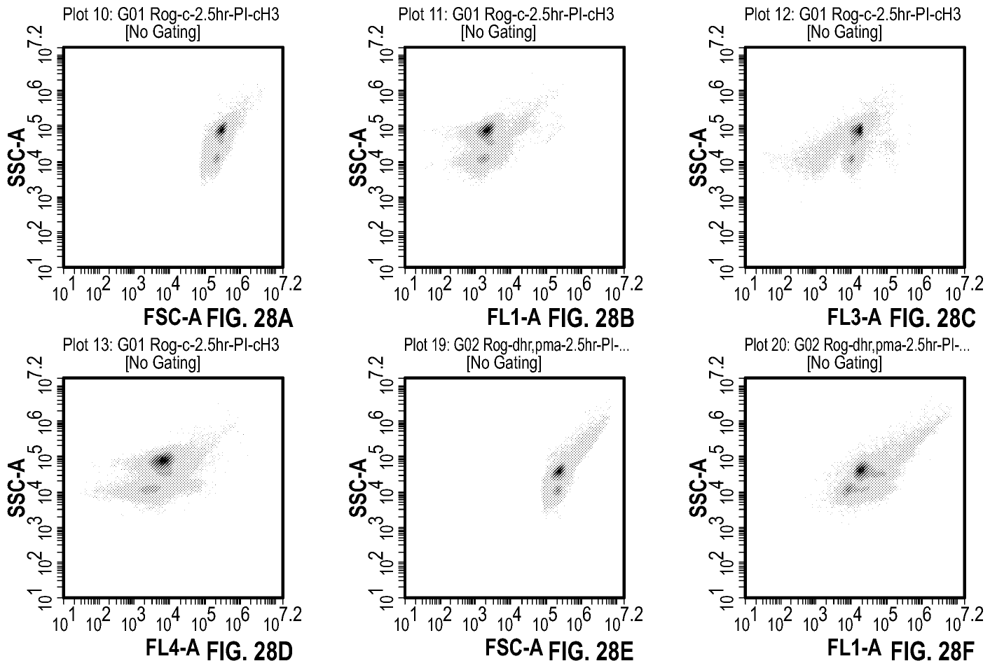

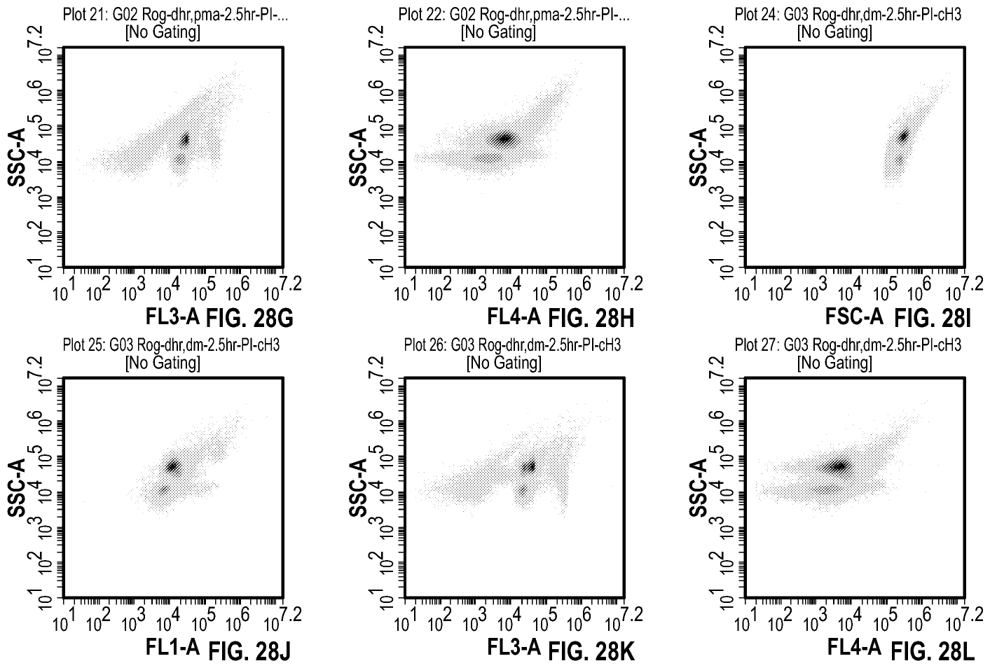

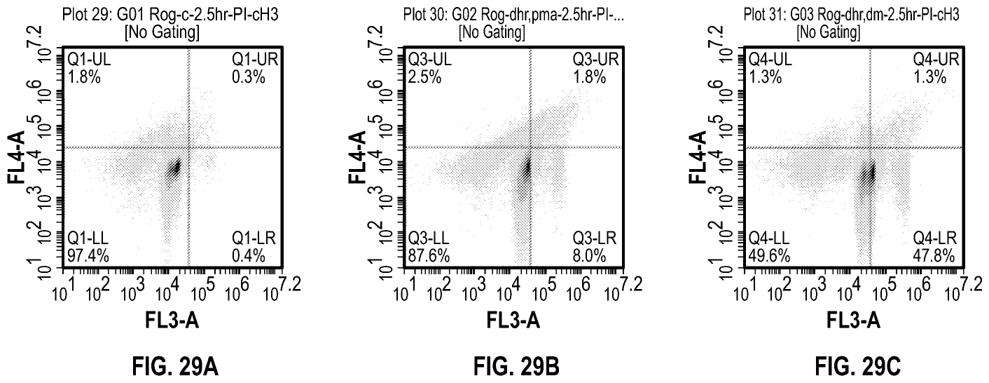

As shown in FIGS. 28A-28L and FIGS. 29A-29C, PMA and dust mite treated groups exhibit higher RH 123 (channel FL1-A) and PI (Channel FL3-A) fluorescence signal, when compared with cell-only group. In addition, the percentage cells positive for citrullinated histone H3 signals was high in these two groups after applying gating strategy on channel FL3-A and FL4-A.

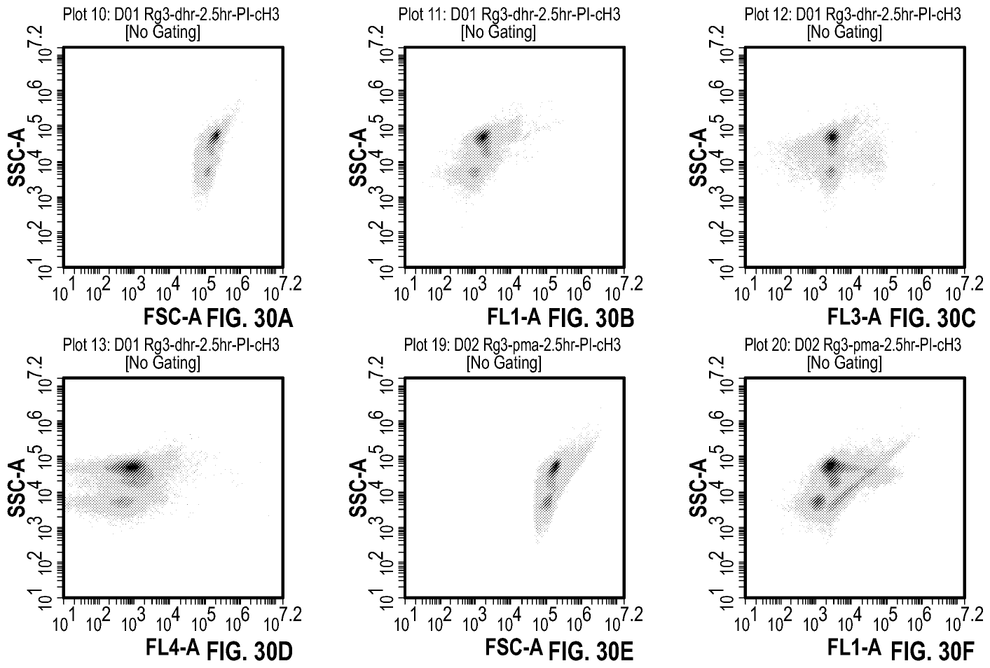

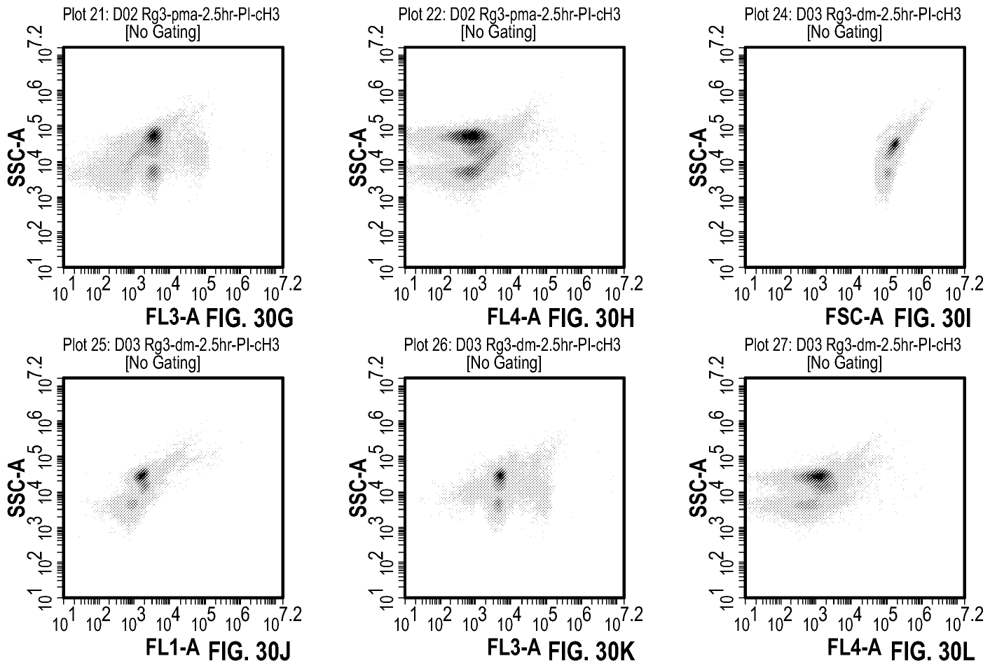

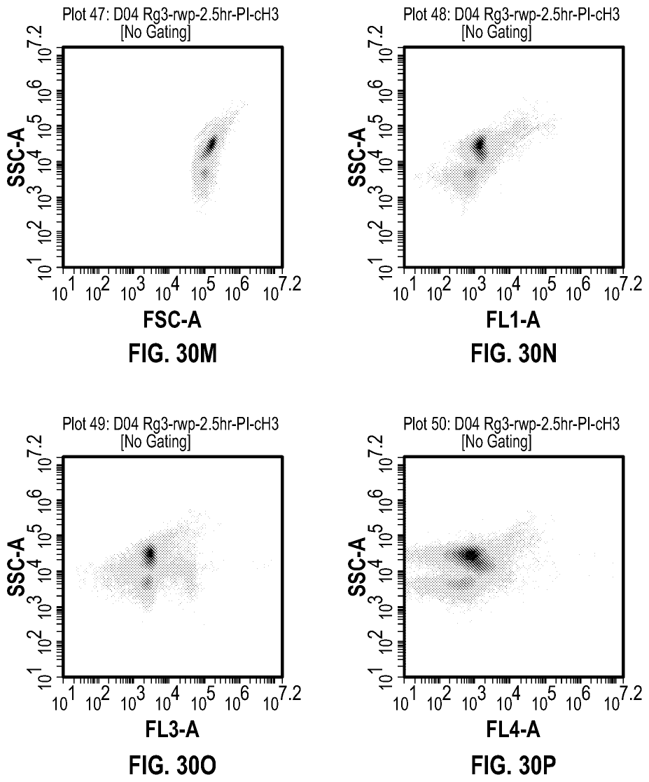

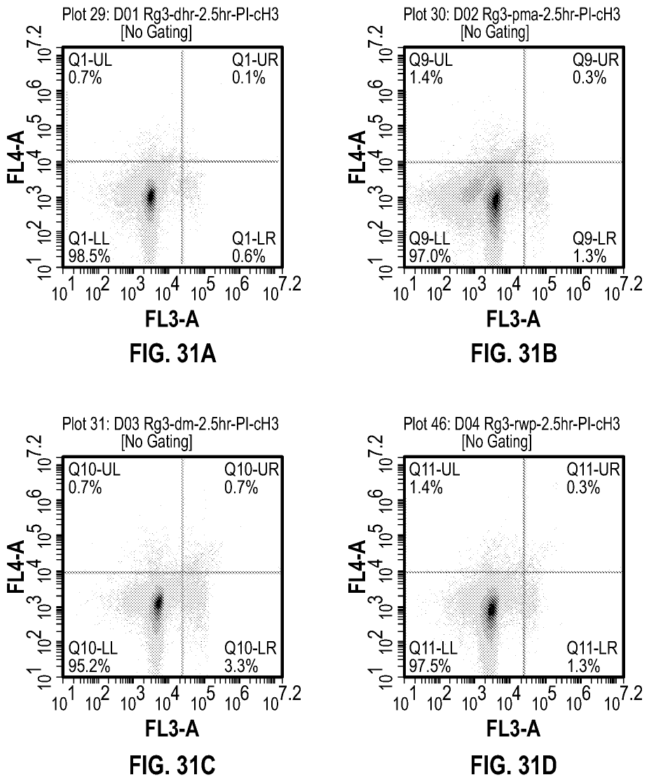

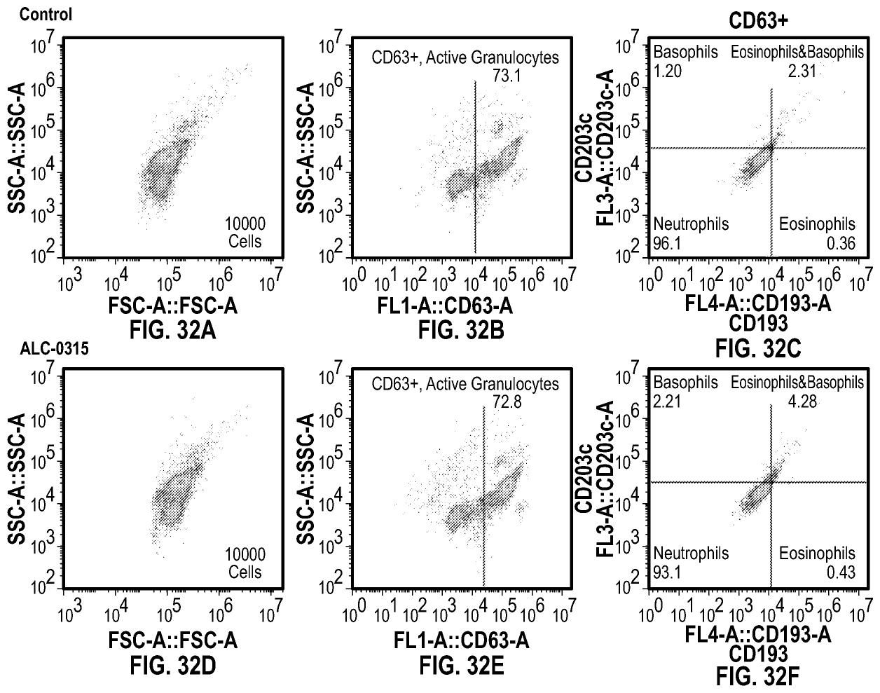

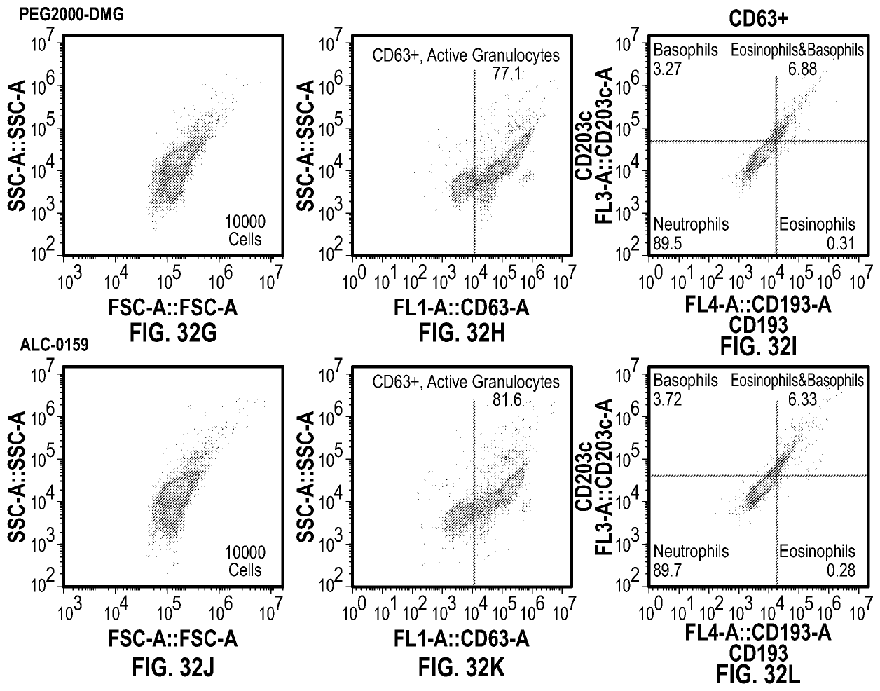

The study was repeated to investigate higher amounts of dust mite and ragweed pollen stimulants (8 μL stimulant/100 μL blood). As shown in FIGS. 30A-30P, the trend that PMA, dust mite stimulant and ragweed pollen stimulant exposed groups exhibited stronger signals continued (See PI staining; channel 3, FL3-A). This result implied extracellular trap formation, especially in the dust mite stimulant-exposed group. Furthermore, the percentage of cells double positive for citrullinated histone H3 signal (Channel 4, FL4-A) and PI signal (Channel 3, FL3-A) was also higher in the dust mite stimulant-exposed group.

### 5.4 Example 4: Granulocyte Reactivity to Exogenous Medicament Stimulants

Incidence of hypersensitivity reactions following vaccination can be difficult to predict, due to discrepancies in reaction status reported by patients along with the inefficiency of current testing platforms to accurately measure allergic reactions. This Example shows application of granulocyte reactivity flow cytometry assay to a biological sample from a human patient that experienced a hypersensitivity reaction after receiving the COVID-19 vaccine and booster (Moderna; 2 doses+booster). While the mechanisms remain unknown, it is believed that this patient has an underlying allergy to one or more vaccine components, such as polyethylene glycol (PEG) and polysorbate 80 (PS80) encapsulated within the lipid nanoparticle, which is the suspected cause of the reported hypersensitivity reaction.

### 5.4.1 Materials and Methods

A human subject with known allergic reaction to mRNA-1273 (Moderna®) COVID-19 vaccine booster was recruited and evaluated for hypersensitivity type I to components in Ad26.COV2.S, mRNA-1273, and BNT162b2 vaccines using the ex vivo methods as described herein (See Table 10 for vaccine components). Total granulocyte activation, along with cell-specific activation (neutrophils, basophils, eosinophils) was measured by CD63, CD11b, CD203c, and CD193 upregulation using flow cytometry, fluorometric microplate analysis, and immunofluorescent microscopy. Analysis of reactive oxygen species (ROS) production was performed using a fluorometric-based detection assay. Quantification of extracellular trap (ET) formation was also performed using the markers citrinullated histone 3 (Cit-H3) and propidium iodide to measure extracellular DNA.

TABLE 10
Vaccine components

| Vaccine | | |
|---|---|---|
| (Manufacturer) | Vaccine Platform | |
| Anaphylaxis Data | (Primary | |
| [Ref] | Component) | Secondary Components |
| BNT162b2 | mRNA-based | (4-hydroxybutyl) azanediyl)bis (hexane-6, 1- |
| (Pfizer-BioNTech) | vaccine; encodes for | diyl)bis(2-hexyldecanoate)] (ALC-0315); |
| ~1:100,000 | spike (S) | 2-[(polyethylene glycol)-2000]-N,N- |
| [Turner et al., World | glycoprotein | ditetradecylacetamide (ALC-0159); |
| Allergy Org J, 2021.] | | 1,2-Diastearoyl-sn-glycero-3-phosphocholine |
| | | cholesterol (DSPC); |
| | | potassium dihydrogen phosphate; |
| | | sodium chloride; |
| | | disodium hydrogen phosphate dihydrate; |
| | | sucrose; |
| | | water |
| mRNA-1273 | mRNA-based | Lipids |
| (Moderna) | vaccine; encodes for | SM-102 |
| none | pre-fusion stabilized | 2,2-dimyristoyl-rac-glycero3- |
| [Turner et al., World | spike (S) | methoxypolyethylene glycol-2000 |
| Allergy Org J, 2021.] | glycoprotein | [PEG2000-DMG] |
| | | cholesterol |
| | | 1,2-diastearoyl-sn- glycero-3-phosphocholine |
| | | [DSPC] |
| | | tromethamine |
| | | tromethamine hydrochloride |
| | | acetic acid |
| | | sodium acetate |
| | | sucrose |
| Ad26.COV2.S | Adenovirus type 26- | 2-hydroxypropyl-β-cyclodextrin (HBCD) |
| (Johnson & Johnson) | based vector; | citric acid monohydrate |
| ~5:10,000 | encodes for spike | ethanol |
| [Shay et al., MMWR, | (S) glycoprotein | hydrochloric acid |
| 2021.] | | polysorbate-80 |
| | | sodium chloride |
| | | sodium hydroxide |
| | | trisodium citrate dihydrate |
| | | water |

Detection of granulocyte activation via flow cytometry, and detection of secreted Cit-H3, DNA, and NET formation via flow cytometry was performed as shown in FIG. 2.

### Fluorometric Analysis of ROS Production and NADPH Oxidase Activity by Plate Reader

Reactive oxygen species (ROS) production and NADPH oxidase activity were measured by means of fluorescent rhodamine 123 (RH 123) as an indicator using the Promega Glomax Multidetection System (Promega, Wisconsin, USA). Peripheral blood samples were collected, diluted, and maintained as previously described. Cells were treated with 5 μg dihydrorhodamine 123 (DHR 123) and incubated at 37° C. under 5% CO₂ for 15 min. Samples were then treated with either 1 M Phorbol-12-Myristate-13-Acetate (PMA), a specific exogenous stimulant, or an equal volume of phosphate-buffered saline (PBS), incubated for an additional 30 min and analyzed in microplates on the Promega Glomax Multidetection System using the blue filter (Ex: 490 nm/Em: 510-570 nm). Data analysis was performed using GraphPad Prism version 9.3.1. ROS Production was quantified as fold-change relative to the mean of the negative control samples.

### ROS Production and NET Detection Via Immunofluorescent Microscopy

Peripheral blood samples were collected and diluted as previously described. 250 μL (˜150,000 cells/mL) of diluted blood containing 5 ug DHR 123 was seeded on coverslips in wells of a 24-well plate and incubated at 37° C. under 5% CO₂ for 15 min to allow for adherence and DHR 123 permeabilization. Cells were stimulated with either 1 M PMA, exogenous stimulant, or PBS, in a volume of 50 μL, for 2-3 hrs to allow NET formation. Final volume of 300 μL/well. For the final 5 min, cells were incubated with 2.5 g of PI. The cells and NETs were fixed using 3.7% formaldehyde and incubated for 10 min at RT. Samples were washed twice with 300 μL PBS for 5 min at RT. The cells were then blocked with 5% FBS for 30 min at RT. For the detection of peptidylarginine deiminase 4 (PAD4)-dependent NET formation, a 1:200 dilution of Cit-H3 (Cayman Chemical) primary antibody was added to the samples and incubated at 4° C. overnight protected from light. Coverslips were washed twice in PBS for 5 min at RT prior to incubation with APC-labelled secondary antibody for 1 h at RT protected from light. Cells were washed twice for 5 min with PBS and coverslips were set to dry on a paper towel. 20 L anti-fade mounting medium with DAPI was added to the center of each coverslip, which was then carefully placed on a clean, sterile, microscope slide with the sample face-down. Samples were set aside briefly until mounting medium covered entirety of the coverslips. Slides were set aside, protected from light, for 30 min to allow for the evaporation of excess mounting medium. Coverslips were sealed and the samples analyzed and stored in 4° C. protected from light to preserve.

5.4.2 Granulocyte Reactivity to BNT162b2, Ad26.COV2.S, and mRNA-1273: Analysis of Hypersensitivity Reaction to COVID-19 Vaccine Components

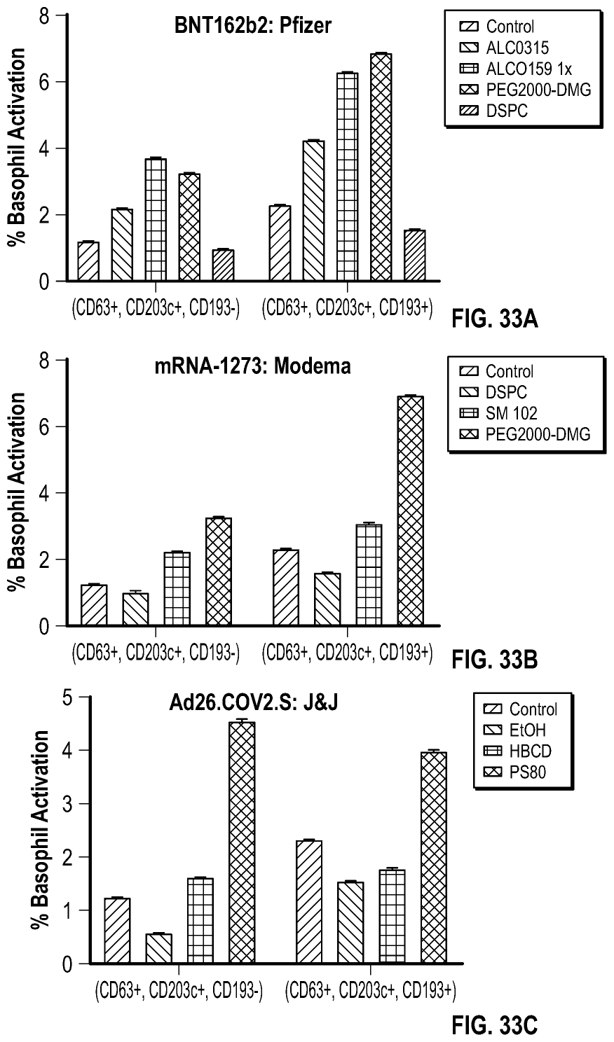

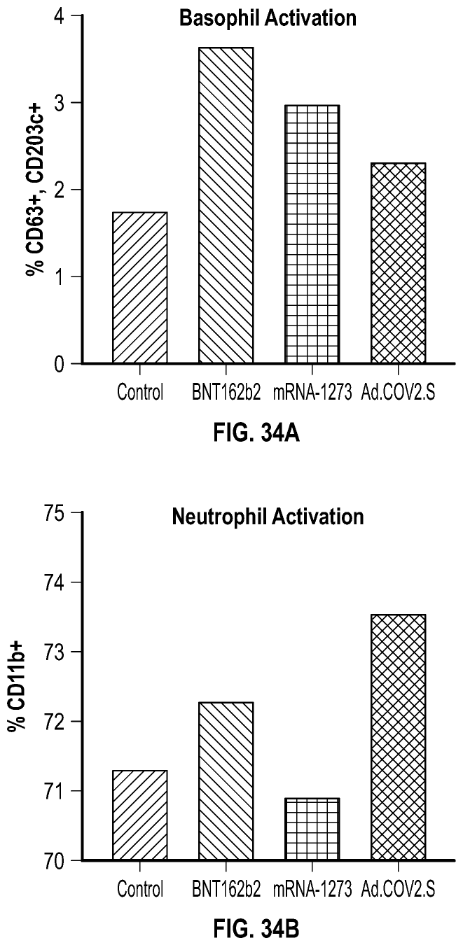

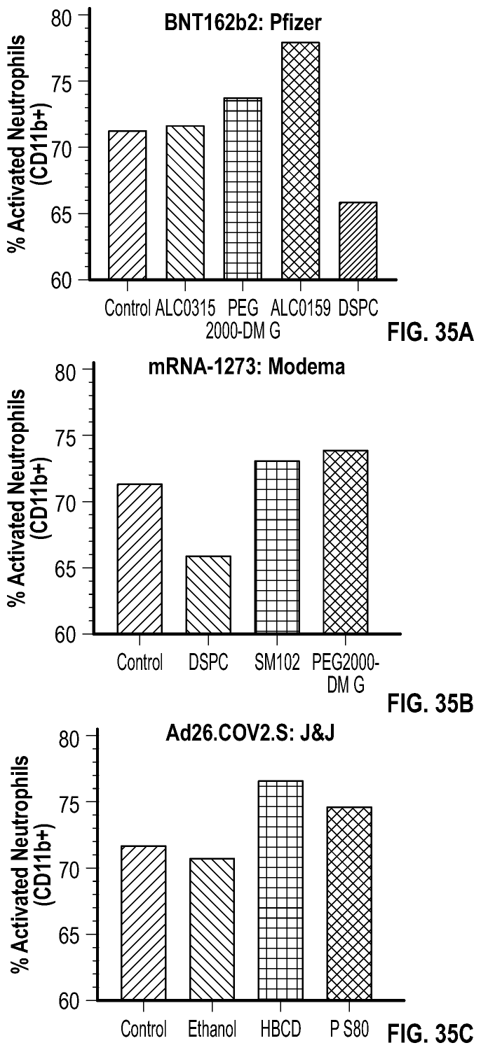

The subject was found to have marked increase in basophil activation and gross granulocyte activation in response to components in the BNT162b2 vaccine (See, e.g., FIG. 33A: ALC0159, ALC0315, and PEG2000-DMG); mRNA-1273 vaccine (See, e.g., FIG. 33B: SM102 and PEG2000-DMG); and Ad26.COV2.S vaccine (See, e.g., FIG. 33C: PS80). Dose-dependent activation of basophils ex vivo was observed. PEGylated lipids but not native PEG produced induction of degranulation. Basophils (CD63+/CD203c+) were activated following exposure to BNT162b2, Ad26.COV2.S, and mRNA-1273 vaccines (FIG. 34A). Neutrophils (CD11b+) were activated following exposure to BNT162b2 and Ad26.COV2.S vaccines (FIG. 34B). Certain components of the vaccines (BNT162b2: ALC0315, PEG2000-DMG, ALC0159; mRNA-1273: SM102, PEG2000-DMG; Ad26.COV2.S: HBCD, PS80) induced higher percent neutrophil activation versus control, whereas certain components did not (i.e., DSPC, ethanol). See FIGS. 35A-35C. Cosensitization was observed to BNT162b2, Ad26.COV2.S, and mRNA-1273 vaccines due to PEGylated components and polysorbate allergy. Taken together, the results support a type I hypersensitivity reaction in subject, measured by basophil activation, along with type III hypersensitivity reaction, measured by increased neutrophil activation. This corresponds with the observed symptoms reported by the patient.

Findings implicate several components in COVID-19 vaccines as potential triggers of anaphylaxis in response to BNT162b2, Ad26.COV2.S, and mRNA-1273, and outline the need for more allergenic hypersensitivity reaction data from patients who are both positive and negative for anaphylactic reactions post-COVID-19 vaccination. These findings also highlight the need for more robust granulocyte activation (e.g., basophil, neutrophil, and eosinophil) detection and monitoring platforms.

What is claimed:

1. A method for detecting or measuring the response of a subject to an exogenous stimulant, said method comprising the use of fluorometric measurement of an amount of oxidation-sensitive fluorophore-treated granulocytes in a biological sample from the subject after contact of the granulocytes with the exogenous stimulant ex vivo,
wherein the method further comprises contacting the oxidation-sensitive fluorophore fluorophore-treated granulocytes with one or more binding moieties that bind to a basophil, eosinophil, or neutrophil, and wherein the one or more binding moieties are detectable by flow cytometry,
and wherein an increase in intensity of fluorescence from an oxidized form of the oxidation-sensitive fluorophore as measured by flow cytometry after contact of the oxidation-sensitive fluorophore-treated granulocytes with the exogenous stimulant as compared to a reference control indicates a response of the subject to the exogenous stimulant.

2. A method for determining or predicting an immune reaction of a subject to an exogenous stimulant, said method comprising the use of fluorometric measurement of an amount of oxidation-sensitive fluorophore-treated granulocytes in a biological sample from the subject after contact of the granulocytes with the exogenous stimulant ex vivo,
wherein the method further comprises contacting the oxidation-sensitive fluorophore fluorophore-treated granulocytes with one or more binding moieties that bind to a basophil, eosinophil, or neutrophil, and wherein the one or more binding moieties are detectable by flow cytometry,
and wherein an increase in intensity of fluorescence from an oxidized form of the oxidation-sensitive fluorophore as measured by flow cytometry after contact of the oxidation-sensitive fluorophore-treated granulocytes with the exogenous stimulant as compared to a reference control indicates an immune reaction of the subject to the exogenous stimulant.

3. The method of claim 2, wherein the predicted immune reaction of the subject to the exogenous stimulant is immune reaction type and/or immune reaction severity.

4. A method for determining or predicting the compatibility of a subject with an exogenous stimulant, said method comprising the use of fluorometric measurement of an amount of oxidation-sensitive fluorophore-treated granulocytes in a biological sample from the subject after contact of the granulocytes with the exogenous stimulant ex vivo,
wherein the method further comprises contacting the oxidation-sensitive fluorophore fluorophore-treated granulocytes with one or more binding moieties that bind to a basophil, eosinophil, or neutrophil, and wherein the one or more binding moieties are detectable by flow cytometry,
and wherein a decrease in or substantially unchanged intensity of fluorescence from an oxidized form of the oxidation-sensitive fluorophore as measured by flow cytometry after contact of the oxidation-sensitive fluorophore-treated granulocytes with the exogenous stimulant as compared to a reference control indicates compatibility of the subject with the exogenous stimulant.

5. The method of any one of claims 1 to 4, wherein the reference control is oxidation-sensitive fluorophore-treated granulocytes in a biological sample from the subject before contact of the granulocytes with the exogenous stimulant ex vivo.

6. The method of any one of claims 1 to 4, wherein the reference control is oxidation-sensitive fluorophore-treated granulocytes in a biological sample from the subject that is not contacted with the exogenous stimulant ex vivo.

7. The method of any one of claims 1 to 6, wherein the oxidation-sensitive fluorophore is selected from dihydrorhodamine (DHR 123), dihydroethidine (DHE), dichlorodihydrofluorscein (DCFH2), or a reduced fluorescein derivative; and/or the oxidized form of the oxidation-sensitive fluorophore is selected from rhodamine, 2-hydroxyethidium, dichlorofluorescein, or an oxidized fluorescein derivative.

8. The method of any one of claims 1 to 7, wherein the oxidation-sensitive fluorophore-treated granulocytes are contacted with one or more binding moieties, wherein each of the one or more binding moieties binds to a different target selected from 2D7, Calprotectin (S100A8/A9), CD9, CD10, CD11a, CD11b, CD11c, CDw12, CD13, CD14, CD15, CD16, CD16b, CD16/32, CD17, CD18, CD22, CD23, CD24, CD25, CD29, CD31, CD32, CD32a, CD32b, CD32c, CD33, CD35, CD37, CD38, CD43, CD44, CD45, CD45RB, CD45RO, CD46, CD47, CD49d, CD50, CD53, CD55, CD58, CD59, CD60a, CD62L, CD63, CD64, CD64a, CD65, CD65s, CD66a, CD66b, CD66c, CD66d, CD68, CD69, CD75S, CD82, CD85A, CD85D, CD85K, CD87, CD88 (C5a receptor), CD89, CD92, CD93, CD95, CD97, CD98, CD100, CD101, CD107a, CD107b, CD112 (Nectin-2), CD114 (G-CSFR), CD116, CD119, CD120a, CD120b, CD123 (IL3Ra), CD125, CD126, CD130, CD131, CD132, CD139, CD141, CD147, CD148, CD153, CD154 (CD40 ligand), CD156a, CD156b, CD157, CD162, CD164, CD170 (SiglecF), CD171, CD172a, CD177, CD178, CD181 (CXCR1), CD182, CD183, CD192 (CCR2), CD193 (CCR3), CD195, CD203c, CD217, CD218, CD218a, CD218b, CD220, CD221, CD222, CD230, CD232, CD244, CD256, CD257, CD258, CD261, CD262, CD263, CD264, CD268, CD270, CD274, CD275, CD281 (TLR1), CD282 (TLR2), CD284 (TLR4), CD286 (TLR6), CD289 (TLR9), CD290, CD294, CD295, CD298, CD302, CD305, CD312, CD314, CD321, CD328, CD329, CD352, CD354, CD360, CD362, C/EBP alpha, CRTH-2, EMR1, FceR1, GATA-2, Ly-6G (Gr-1), or Siglec-8.

9. The method of any one of claims 1 to 8, wherein the oxidation-sensitive fluorophore-treated granulocytes are contacted with one or more binding moieties, wherein each of the one or more binding moieties binds to a different target selected from CD11b, C16, C16b, CD33, CD63, CD123, CD193, or CD203c.

10. The method of any one of claims 1 to 9, wherein the oxidation-sensitive fluorophore-treated granulocytes are basophils, and the basophils are contacted with one or more binding moieties that each bind to a different target selected from 2D7, CD9, CD11a, CD11b, CD13, CD15, CD16, CD16/32, CD22, CD25, CD32, CD33, CD38, CD43, CD45, CD49b, CD63, CD69, CD88 (C5a receptor), CD123 (IL3Ra), CD125, CD154 (CD40 ligand), CD192 (CCR2), CD203c, CD218 (IL-18R), CD282 (TLR2), CD284 (TLR4), CD286 (TLR6), CD294 (CRTH2), CD281 (TLR1), CD289 (TLR9), C/EBP alpha, CRTH-2, FceR1, or GATA-2; optionally selected from CD16, CD63, CD123, or CD203c.

11. The method of any one of claims 1 to 10, wherein the oxidation-sensitive fluorophore-treated granulocytes are eosinophils, and the eosinophils are contacted with one or more binding moieties that each bind to a different target selected from CD9, CD11b, CD13, CD15, CD16, CD16/32, CD24, CD32, CD33, CD35, CD43, CD45, CD49d, CD63, CD64, CD66b, CD116, CD123, CD125, CD126, CD170 (SiglecF), CD193 (CCR3), CD244, EMR1, FceR1, or Siglec-8; optionally selected from CD16, CD63, or CD193.

12. The method of any one of claims 1 to 11, wherein the oxidation-sensitive fluorophore-treated granulocytes are neutrophils, and the neutrophils are contacted with one or more binding moieties that each bind to a different target selected from Calprotectin (S100A8/A9), CD10, CD11b, CD13, CD15, CD16, CD16/32, CD17, CD18, CD24, CD32, CD33, CD35, CD43, CD44, CD49d, CD63, CD66a, CD66b, CD66c, CD66d, CD89, CD93, CD112 (Nectin-2), CD114 (G-CSFR), CD116, CD123, CD157, CD177, CD181 (CXCR1), CD193, CD281 (TLR1), CD282 (TLR2), CD284 (TLR4), CD286 (TLR6), CD289 (TLR9), or Ly-6G (Gr-1); optionally selected from CD11b, CD16, CD63, CD123, or CD193.

13. The method of any one of claims 1 to 12, wherein the oxidation-sensitive fluorophore-treated granulocytes are contacted with an antibody that detects granulocyte degranulation.

14. The method of any one of claims 1 to 13, wherein the oxidation-sensitive fluorophore-treated granulocytes are contacted with an antibody that binds to CD63.

15. The method of any one of claims 1 to 14, wherein the method further comprises staining the oxidation-sensitive fluorophore-treated granulocytes to determine viability of the granulocytes.

16. The method of any one of claims 1 to 15, wherein the method further comprises staining the oxidation-sensitive fluorophore-treated granulocytes to determine positive extracellular trap formation.

17. The method of claim 15 or 16, wherein the viability and/or positive extracellular trap formation is determined by propidium iodide (PI) staining.

18. The method of any one of claims 1 to 17, wherein the method further comprises contacting the oxidation-sensitive fluorophore-treated granulocytes with an anti-citrullinated histone H3 (H3cit) antibody to determine extracellular trap formation, wherein the anti-H3cit antibody is detectable by flow cytometry.

19. The method of any one of claims 1 to 18, wherein the exogenous stimulant is an environmental stimulant, a foodstuff stimulant, or a medicament stimulant.

20. The method of claim 19, wherein the environmental stimulant is environmental allergen selected from pollen, plant, insect, dust mite, cockroach, animal, venom, mold, latex, metal, vitamin, or mineral.

21. The method of claim 19, wherein the foodstuff stimulant is a foodstuff allergen selected from seed, nut, egg, dairy product, oil, condiment, fruit, vegetable, cereal, grain, legume, meat, wheat, soy, seafood, herb, or spice.

22. The method of claim 19, wherein the medicament stimulant is selected from a drug compound, vaccine, adjuvant, or pharmaceutical excipient.

23. The method of any one of claims 1 to 22, wherein the biological sample is selected from a buccal swab sample, bronchoalveolar lavage sample, saliva sample, sputum sample, urine sample, or blood sample, optionally wherein the blood sample is a peripheral blood sample.

24. The method of claim 23, wherein the blood sample is about 1 mL or less, about 500 μL or less, about 400 μL or less, about 200 μL or less, about 150 μL or less, about 125 μL or less, about 100 μL or less, about 75 μL or less, or about 50 μL or less.

25. The method of any one of claims 1 to 24, wherein the granulocytes are treated with from about 1 μg/mL to about 5 μg/mL of DHR 123.

26. The method of any one of claims 1 to 25, wherein the granulocytes are contacted with from about 0.3 μg/mL to about 3 μg/mL of exogenous stimulant.

27. The method of any one of claims 1 to 26, wherein the red blood cells in the biological sample are lysed after contact with the exogenous stimulant.

28. The method of any one of claims 1 to 27, wherein the granulocytes in the biological sample are fixed after contact with the exogenous stimulant.

29. The method of claim 27 or 28, wherein the granulocytes in the biological sample are contacted with the one or more binding moieties that binds to a basophil, eosinophil, or neutrophil after lysis of the red blood cells and/or after fixation of the granulocytes.

30. The method of any one of claims 1 to 29, wherein the granulocytes are identified by flow cytometry detection of the labeled antibody, either alone or in combination with side scatter analysis.

31. The method of any one of claim 1 to 30, wherein the method is completed in about 3 hours or less, about 2.5 hours or less, about 2.0 hours or less, about 1.5 hours or less, about 1.0 hours or less, or about 0.5 hours or less.

32. The method of any one of claims 1 to 31, wherein the intensity of fluorescence is increased by at least about 10%, at least about 20%, at least about 30%, at least about 40%, at least about 50%, at least about 60%, at least about 70%, at least about 80%, at least about 90%, at least about 2-fold, at least about 3-fold, at least about 4-fold, or at least about 5-fold as compared to the reference control.

33. The method of any one of claims 1 to 32, wherein the antibody comprises a label detectable by flow cytometry, wherein the label is a fluorophore.

34. The method of claim 33, wherein when more than one antibody detectable by flow cytometry is used in the method, the label for each antibody is different.

35. The method of any one of claims 1 to 34, wherein the antibody detectable by flow cytometry comprises a binding site for a secondary antibody, wherein the secondary antibody is detectable by flow cytometry or is a binding site for a tertiary antibody that is detectable by flow cytometry.

36. The method of any one of claims 1 to 35, wherein the fluorometric measurement is by flow cytometry or plate reader.
